(19) United States
(12) Patent Application Publication
(10) Pub. No.: US 2024/0428881 A1
(43) Pub. Date: Dec. 26, 2024

(54) DETERMINATION OF LYMPHOCYTE ABUNDANCE IN MIXED SAMPLES

(71) Applicants: THE FRANCIS CRICK INSTITUTE, London (GB); UNIVERSITY COLLEGE LONDON, London (GB)

(72) Inventors: Robert BENTHAM, London (GB); Thomas Benjamin Kingdon WATKINS, London (GB); Nicholas McGranahan, London (GB); Charles Swanton, London (GB)

(21) Appl. No.: 18/416,969

(22) Filed: Jan. 19, 2024

Related U.S. Application Data
(63) Continuation of application No. PCT/EP2022/070694, filed on Jul. 22, 2022.

(30) Foreign Application Priority Data
Jul. 22, 2021 (GB) 2110555.6
Mar. 11, 2022 (GB) 2203451.6

(51) Int. Cl. G16B 20/00; G16B 40/20

(52) U.S. Cl. CPC G16B 20/00; G16B 40/20

(57) ABSTRACT

Methods for determining the fraction of lymphocytes in a mixed sample comprising genomic material from multiple cell types are described. The methods comprise obtaining a read depth profile for the sample along a predetermined genomic region of interest including at least a portion of a genomic locus that undergoes VDJ recombination; obtaining a plurality of read depth ratios (rᵢ) by normalising the read depths by reference to a baseline read depth derived from a subset of the region of interest; obtaining a summarised read depth ratio value (r_{VDJ}) for a subset of the region of interest that is likely to be deleted through VDJ recombination; and determining the fraction of lymphocytes (f) in the sample as a function of the summarised read depth ratio value (r_{VDJ}). Methods of providing a diagnosis or prognosis based on the lymphocyte fraction are also described, as well as related systems and products.

### FIELD OF THE DISCLOSURE

The present disclosure relates to methods of estimating the fraction of lymphocytes, such as T lymphocytes or B lymphocytes, in mixed samples comprising multiple cell types, such as tumour samples or blood samples, based on read depth profiles derived from the samples, and to systems and related products for implementing such methods. Also described are methods of providing a prognosis for a cancer patient based at least in part on the estimated tumour infiltrating lymphocyte fraction from a tumour sample from the patient.

### BACKGROUND

Lymphocytes are essential parts of the human system that play a crucial part in human health and disease. For example, in the case of tumours, the immune system is widely recognised as one of the key factors that influence both tumorigenesis and subsequent evolution of cancer. T cells in particular play an important part of the cancer immune microenvironment by eliminating cancer cells that harbour neoantigens, and the number of T cells within a tumour sample is therefore an important clinical factor. Indeed, the status of the immune microenvironment of tumours at clinical presentation can be both prognostic and predictive of response to immunotherapy. In recent years immunotherapy and particularly checkpoint inhibitors (CPI) has emerged as a revolutionary treatment for many cancer types. Indeed, melanoma and non-small cell lung cancer (NSCLC) have shown remarkable improvements in both disease-free and overall survival following either anti-CTL4 or anti-PDL1 therapy (Robert et al., 2011; Schadendorf et al., 2015; Topalian et al., 2012). Response to immunotherapy however is not universal, with clinical beneficial response only occurring in a subset of patients (Goodman et al., 2017). Determining which patients will benefit from CPI is therefore of critical importance.

Emerging data suggests response to CPI is principally governed by two features; an immune stimulus, such as presence of neoantigens that can be predicted from next generation sequencing (NGS), and the presence of T cells which are able to respond to the stimulus. The presence of infiltrating T cells has long been associated with improved survival in cancer types, such as in ovarian cancer (Zhang et al., 2003) and colorectal cancer (Galon et al., 2006). More recent research has focused on the predictive potential of neoantigens, with increased tumour mutational burden (TMB) emerging as one of the best predictors of response to immunotherapy (Samstein et al., 2019). A recent study by Litchfield et al (2020) systematically investigated the degree to which different potential biomarkers of CPI response perform in a pan-cancer CPI treated dataset. Two of the most predictive features identified were clonal TMB, reflecting mutations present in every cancer cell and RNA sequencing derived transcriptomic signatures for T cells (e.g. CD8A RNA expression), signifying presence of tumour infiltrating lymphocytes (TILs).

Thus, estimating both the presence and abundance of TILs is of vital importance in cancer treatment. However, currently there is no universal state-of-the-art method for estimating TILs. Microarray signatures such as CIBERSORT (Newman et al., 2015) as well as RNA-seq signatures such as CIBERSORTx for RNA-seq (Newman et al., 2019), ESTIMATE (Yoshihara et al., 2013) and those compiled by Danaher et al., have been used to quantify the transcriptomic signatures of TILs in tumour samples. Alternatively, the presence of immune infiltrate can be determined based haematoxylin eosin on and (H&E) histopathological slides or alternatively with the appropriate cell-specific staining, most commonly immunohistochemistry. f more phenotypic knowledge of the T cells is required, T cell receptor (TCR) sequencing can be performed following enrichment for those sequences encoding the highly diverse VDJ recombined CDR3 region of the TCR (Bolotin et al., 2012). All of these methods require the acquisition of dedicated data for the purpose of identifying immune cells in mixed samples such as tumour samples. More recently, McGrath et al. (2017) proposed an approach to characterise the TCR repertoire in a sample using WES data by isolating T cell rearranged reads (reads that span the hypervariable CDr3 and include both V and J segments). This approach however requires extremely high sequencing depth, is unable to deal with the presence of aneuploidy in the non-T cell population (as is frequent e.g. in the case of cancer), and is only suitable to characterise the TCR repertoire.

Thus, there exists a need for new methods for determining the abundance of lymphocytes in mixed samples, that alleviate one or more of the drawbacks of existing methods.

### BRIEF DESCRIPTION OF THE INVENTION

Broadly, the present inventors recognised that whole exome sequencing (WES) or even whole genome sequencing (WGS) is frequently performed on tumour samples to calculate tumour mutational burden and to identify actionable mutations for targeted therapy. Similarly, this type of data is increasingly frequently collected in other disease contexts such as neonatal care. However, at present WES cannot be used to accurately infer immune infiltrate levels. Thus, the inventors develop a method for T cell (or B cell) fraction estimation from WES/WGS or similar data, using a signal derived from T cell receptor excision circles (TRECs) during VDJ recombination, such as e.g. VDJ recombination of the T cell receptor alpha (TCRA) gene. This score directly measures the proportion of T cells (or B cells, as the case may be) in a sample. The inventors further demonstrated that this score significantly correlates with orthogonal immune infiltrate scores based on RNA sequencing and histopathological TIL scoring in the TRACERx100 cohort. Using a meta-analysis of patient cohorts receiving immunotherapy, the inventors confirmed that this score was predictive of response to cancer immunotherapy, providing predictive value beyond mutational burden. The inventors further showed the broad applicability of the method by demonstrating that the T cell fraction can also be calculated from blood derived germline samples. Thus, the method provides a window into the immune microenvironment of a WES (or WGS) tumour sample, which is particularly useful where orthogonal data is not available.

Accordingly, in a first aspect the present disclosure provides a method for determining the fraction of lymphocytes in a mixed sample comprising genomic material from multiple cell types, the method comprising: obtaining a read depth profile for the sample comprising the read depths along a predetermined genomic region of interest, wherein the predetermined genomic region of interest includes at least a portion of a genomic locus that undergoes VDJ recombination; obtaining a plurality of read depth ratios (rᵢ) by normalising the read depths along the predetermined region of interest by reference to a baseline read depth derived from a subset of the region of interest; obtaining a summarised read depth ratio value (r_{VDJ}) for a subset of the region of interest that is likely to be deleted through VDJ recombination; and determining the fraction of lymphocytes (f) in the sample as a function of the summarised read depth ratio value (r_{VDJ}).

The method may have any one or more of the following optional features.

The mixed sample may be a cell or tissue sample (in which case the sample may comprise multiple cell types each comprising respective genomic material), or a sample of nucleic acid derived therefrom.

A genomic locus that undergoes VDJ recombination may be selected from the TCRA, TCRB, TCRG, TCRD, IGH, IGL or IGK gene locus. Accordingly, the fraction of lymphocytes may be a fraction of T lymphocytes, a fraction of B lymphocytes, a fraction of αβ T lymphocytes, a fraction of γδ T lymphocytes, a fraction of B cells or T cells including any gene segment in the TCRA, TCRB, TCRG, TCRD, IGH, IGL or IGK gene locus, or a fraction of B cells or T cells including any gene segments listed in Table 8 or corresponding gene segments in another species (where the coordinates listed in Table 8 are indicative only and in particular corresponding coordinates in another reference sequence may be used). A genomic locus that undergoes VDJ recombination may be selected from the TCRA, TCRB, TCRG, or IGH gene locus. The predetermined region of interest may include one or more exons from the TCRA, TCRB, TCRG, TCRD, IGH, IGL or IGK gene locus. The predetermined region of interest may include one or more exons from the TCRA, TCRB, TCRG, or IGH gene locus. The predetermined region of interest may include a plurality of exons from the TCRA, TCRB, TCRG, TCRD, IGH, IGL or IGK gene locus, wherein the plurality of exons comprise at least one exon corresponding to each of a V, D and C segment of the respective gene locus. The plurality of exons may comprise at least one exon corresponding to each of a V, D, J and C segment of the respective gene locus. The plurality of exons may comprise exons corresponding to multiple V segments of the respective gene locus. The predetermined region of interest may include the regions coding for a plurality of V, D, J and/or C segments from the TCRA, TCRB, TCRG, TCRD, IGH, IGL or IGK gene locus. The predetermined region of interest may include one or more regions located between V, D, J and/or C segments from the TCRA, TCRB, TCRG, TCRD, IGH, IGL or IGK gene locus. The predetermined region of interest may include all exons from the TCRA, TCRB, TCRG, TCRD, IGH, IGL or IGK gene locus. The predetermined region of interest may include all of the TCRA, TCRB, TCRG, TCRD, IGH, IGL or IGK gene locus (i.e. including both exons and introns).

The predetermined region may include a subset of the exons from the TCRA, TCRB, TCRG, TCRD, IGH, IGL or IGK gene locus. The predetermined region may include a subset of the TCRA, TCRB, TCRG, TCRD, IGH, IGL or IGK gene locus. For example, the predetermined region may exclude any exon or region that has been determined to be associated with a systematic bias, such as e.g. a bias due to the platform used to collect the read data, a bias associated with the GC content in the exon or region, or a bias associated with the coverage in the exon or region. For example, some exon capture kits have been shown to be associated with biases that lead to coverage in certain exons departing from the expected. Similarly, certain regions may have lower or higher coverage than expected in whole genome sequencing for example due to artefacts in the sequencing or alignment process. Such exons or regions may be identified by comparing read coverages between datasets that are not expected to be subject to the same systematic bias, such as e.g. acquired using different platforms (e.g. different capture kits). For example, the average log R ratio for a plurality of candidate exons or regions may be calculated for a first set of samples and a second set of samples (where the two sets of samples are not expected to suffer from the same systematic bias), and the median across each set of samples may be compared for each of the plurality of candidate exons or regions to identify those that differ significantly between the two sets of samples. For example, a difference between median log R above a threshold, such as e.g. 0.5 may be used as a criterion to exclude exons/regions as likely to be subject to bias. Regions with lower or higher coverage than expected may be identified by fitting a model to the mean read depth ratio data across a plurality of samples, such as e.g. a general linear model, and removing any region of a predetermined size (such as e.g. 100 bp, 200 bp, 500 bp, 1000 bp) that is associated with a summarised value (e.g. a mean or median value) that is not within a predetermined distance of the fitted model. Instead or in addition to this, regions with lower or higher coverage than expected may be identified by determining a fraction of lymphocytes for a plurality of samples and performing a genome wide association study to identify single nucleotide polymorphisms that are associated with the determined fraction of lymphocytes, where the presence of an association is indicative of coverage bias in the region of predetermined size comprising the single nucleotide polymorphism. Thus, the method may comprise identifying regions with lower or higher coverage than expected in whole genome sequencing by determining a fraction of lymphocytes for a plurality of samples, performing a genome wide association study to identify single nucleotide polymorphisms that are associated with the determined fraction of lymphocytes, selecting samples including the single nucleotide polymorphism, fitting a model to the mean read depth ratio data across the selected samples, and removing any region of a predetermined size (such as e.g. 100 bp, 200 bp, 500 bp, 1000 bp) that is associated with a summarised value (e.g. a mean or median value) that is not within a predetermined distance of the fitted model.

The read depth or read depth ratios may be processed to correct for biases associated with the read depth data acquisition platform used. For example, the read depth/read depth ratios may be GC corrected. GC correction of read depth data may be performed using a linear model capturing the influence of GC content on read depth at every position, where the residuals of the model represent the normalised read depth values. The influence of GC content on read depth may be represented by a term that reflects GC content at the exon or region level (GCₑₓₒₙ/GC_{region}) where a region may be defined as a window of a predetermined size such as e.g. 1000 bp (in which case GC_{region} may be referred to as “GC_{1000 bp}”) and a term that reflects the GC content at the level of the entire predetermined region of interest (GC_{macro}, obtained from a model, for example a linear model, fitted to smoothed data such as for example using 1000 bp windows along the predetermined region of interest). For example, a linear model of the form lm(basepair˜GCₑₓₒₙ+GC²ₑₓₒₙ+GC_{macro}+GC²_{macro}) Or equivalently lm(basepair˜GC_{region}+GC²_{region}+GC_{macro}+GC²_{macro}) (such as in particular lm(basepair˜GC_{1000 bp}+GC²_{1000 bp}+GC_{macro}+GC²_{macro})) may be used, where “basepair” is the read depth at every position. The residuals of such a model may be used as the corrected coverage values. The corrected (e.g. GC corrected) read depth values may be used to determine the baseline read depth derived from a subset of the region of interest and the summarised read depth ratio value (r_{VDJ}) for the subset of the region of interest that is likely to be deleted through VDJ recombination. For example, the median value (or other summarised metric such as e.g. a statistical measure of centrality) of the corrected values at the respective subset of the region of interest may be used. Where the subset of the region of interest comprises a plurality of genomic locations (such as e.g. the beginning and end of the region likely to undergo VDJ recombination), one of the plurality of median values may be used, such as e.g. the highest one. A read depth profile may be obtained from sequencing data, preferably from whole exome sequencing, whole genome sequencing (including low pass WGS, also referred to as shallow genome sequencing), or panel sequencing data. When the read depth profile is obtained from panel sequencing data, the panel sequencing data comprises data for the predetermined genomic region of interest. For example, the data may have been obtained using capture probes that target a plurality of genomic regions including the predetermined genomic region of interest. The read depth profile may be obtained from sequencing data that has a sequencing depth of at least 0.1×, at least 0.5×, at least 2×, at least 5×, at least 10×, preferably at least 15×, preferably at least 20×, more preferably at least 30×. The read depth profile may be obtained from whole exome sequencing data that has a sequencing depth of at least 10×, preferably at least 15×, preferably at least 20×, more preferably at least 30×. The read depth profile may be obtained from whole genome sequencing data that has a sequencing depth of at least 0.1×, at least 0.5×, at least 2×, at least 5×, or at least 10×, preferably at least 2×. The read depth profile may be obtained from sequencing data that has been filtered to remove exons that have a sequencing depth below a threshold, such as e.g. 10×, preferably 15×. The read depth profile may be obtained from sequencing data that has been filtered to remove regions (e.g. defined using a window of fixed size such as e.g. 1000 bp) that have a sequencing depth below a threshold, such as e.g. 2×. Sequencing data comprising fewer than a predetermined number of exons in the predetermined genomic region of interest with a sequencing depth above a threshold (such as e.g. 10× or 15×) may be discarded. For example, data comprising fewer than 30 exons in the predetermined region of interest (e.g. the TCRA locus) with a sequencing depth above a threshold (e.g. 15×) may be discarded. In such cases, new sequence data may be acquired for the sample, or the sequence data may be sued to investigate another predetermined genomic region of interest where the data has sufficient coverage.

The summarised read depth value may be a summarised log read depth ratio. The log may be a base 2 log. Determining the fraction of lymphocytes (f) in the sample as a function of the summarised read depth ratio value (r_{VDJ}) may comprise determining the fraction of lymphocytes (f) in the sample as a function of the summarised read depth ratio value (r_{VDJ}), the fraction of abnormal cells (p) in the mixed sample, where abnormal cells are those that are aneuploid in the predetermined region of interest, and the copy number of the abnormal cells in the predetermined region of interest (Ψ_{T}). The fraction of abnormal cells (p) in the mixed sample, and the copy number of the abnormal cells in the predetermined region of interest (Vr) may be determined by obtaining an abnormal cell-specific copy number profile using methods known in the art, such as e.g. the ASCAT method described in Van Loo et al. or the pureCN method described in Riester et (2016) al. and available at http://bioconductor.org/packages/PureCN/. Determining the fraction of lymphocytes (f) in the sample may comprise determining the value of f as:

$$f = ( 1 - p + \frac{p}{2} {Ψ}_{T} ) - ( 1 - p + \frac{p}{2} {Ψ}_{T} ) {2}^{\frac{{r}_{VDJ}}{γ}} \qquad ( 7 )$$

where γ is a constant. Determining the fraction of lymphocytes (f) in the sample may comprise determining the value of f as:

$$f = 1 - {2}^{\frac{{r}_{VDJ}}{γ}} \qquad ( 3 )$$

where γ is a constant. The parameter γ may be adjusted dependent on the analytic platform used to obtain the read depth data. For example, when using Illumina Hiseq, the value of γ may be=1 (i.e. the parameter can be removed from all equations). Without wishing to be bound by theory, it is believed that the parameter γ captures platform-related “compaction” of log R profiles (also referred to as ‘r’, log R is the log ratio of read depths in a region of interest vs a reference region, in the present case) compared with theoretically expected values. For example, r should be equal to zero when comparing regions expected to have the same copy numbers. Thus, this parameter may be set for a particular platform by comparison with expected values in control settings. As the skilled person understands, equation (3) is equivalent to equation (7) if W=2 and/or p=0 (i.e. the sample does not contain abnormal cells, or any cells in the sample that may be considered abnormal by reference to other regions are not aneuploid in the region of interest). Thus, equation (3) may be used when the sample is not expected to contain abnormal cells (e.g. a germline sample). Equation (3) may also be used when p and/or Wr are unknown, uncertain or unreliable (i.e. when the proportion of abnormal cells and/or their average copy number in the predetermine region of interest is unknown, uncertain or unreliable). For example, equation (3) may be used if the result of equation (7) exceeds 1-p. Without wishing to be bound by theory, the inventors believe that equation (3) will be suitable even if in fact the sample does contain abnormal cells, as long as the lymphocytes represent a relatively small proportion of the cells in the mixed sample.

The subset of the region of interest that is likely to be deleted through VDJ recombination may comprise a region located between the end of the last V segment of the genomic locus that undergoes VDJ recombination, and the start of the J segment of the genomic locus that undergoes VDJ recombination. The subset of the region of interest that is likely to be deleted through VDJ recombination may comprise one or more D segments (such as e.g. all D segments) of the genomic locus that undergoes VDJ recombination. The subset of the region of interest that is likely to be deleted through VDJ recombination preferably does not include any V or C segment. Instead or in addition to this, the subset of the region of interest that is likely to be deleted through VDJ recombination preferably does not include any J segment. The subset of the region of interest that is likely to be deleted through VDJ recombination may comprise, consist of, or be comprised in a gap between the final V gene segment and a first segment that encodes a J segment of the genomic locus that undergoes VDJ recombination, or in the case of TCRA a first segment that encodes a part of the TCRδ chain, or any corresponding segment in another predetermined region of interest. For example, when looking at the TCRA locus, the present inventors have found a definition of the location of maximum VDJ recombination as the gap between the final TCRA V segment and the first segment of the TCR delta gene, rounded to have a size of 80000 bp and start at position 22800000 (leading to the region chr14: 22800000-22880000) to be suitable. Other definitions are possible and small changes in the size of this region are not expected to negatively impact the performance of the method. Regions of maximum VDJ recombination may be defined for other VDJ loci using a similar approach, i.e. by defining a region located at least partially around or within the region between the final V gene segment and the first J gene segment. Regions of maximum VDJ recombination may be defined for respective VDJ loci using the genomic coordinates in Table 7, or corresponding coordinates for other species or reference genomes. The subset of the region of interest that is likely to be deleted through VDJ recombination may comprise a region corresponding to any V or J segment of the genomic locus that undergoes VDJ recombination. This may be used when the fraction of lymphocytes is a fraction of B cells or T cells including any gene segment in the TCRA, TCRB, TCRG, TCRD, IGH, IGL or IGK gene locus, or a fraction of B cells or T cells including any gene segment listed in Table 8 or corresponding gene segments in another species.

The method may further comprise fitting a model to the plurality of read depth ratios, and obtaining a summarised read depth ratio value (r_{VDJ}) for a subset of the region of interest that is likely to be deleted through VDJ recombination may comprise obtaining a summarised read depth ratio value based on the value of the model in the subset of the region of interest that is likely to be deleted through VDJ recombination. The model may capture the log read depth ratio as a function of the genome position along the predetermined genomic region of interest. The plurality of read depth ratios may also be referred to herein as a “read depth ratio profile”. As the skilled person understands, the plurality of read depth ratios are organised relative to each other along genomic coordinates and as such reference to fitting a model to this plurality of value refers to fitting model to the data series comprising the plurality of read depth ratios as a function of genomic coordinates. The model may be a generalised linear model, such as a generalised additive model, a piecewise constant model, or a Bayesian model. The model may be a piecewise constrained linear model with a plurality of breakpoints selected to correspond to locations of a plurality of V and J genes in the region of interest. For example, the model may be a piecewise constrained linear model with a plurality of breakpoints selected from the starts and ends of the genes listed in Table 8, from the genomic locations listed in Table 8, or from corresponding genes or locations in other species or reference genomes. The model may be a general linear model and the sequence data may have been obtained using a panel capture method such as whole exome sequencing. The model may be a piecewise constrained linear model and the sequence data may have been obtained using whole genome sequencing. A piecewise constrained linear model may have one or more (or all) of the following further constraints: the model may have a value of 0 before the first V segment in the predetermined genomic region of interest, the model may have a value of 0 after the last J segment in the predetermined genomic region of interest, the model may be such that the value of each subsequent V segment in the predetermined genomic region of interest has a value that is lower than that of the preceding segment along genomic coordinates, the model may be such that the value of each subsequent J segment in the predetermined genomic region of interest has a value that is higher than that of the preceding segment along genomic coordinates. Any approach that fits a smoothed curve (whether continuous or piecewise constant) to the data may be used in the context of the disclosure. As another example, the model may be a Bayesian modelling segment usage from the read depth and a prior distribution of usage of each of a plurality of V and J genes in the region of interest. A prior distribution of usage of each of a plurality of V and J genes in the region of interest may be a uniform distribution. Such a distribution may define an equal likelihood for each gene to be used. A prior distribution of usage of each of a plurality of V and J genes in the region of interest may be a distribution using prior knowledge of V and J gene (segment) usage in a reference population. For example, T cell receptor sequencing data obtained from a reference population could be used to determine the distribution of V and J gene usage in the reference population. The reference population may be a human population. The reference population may be a human population with one or more specific characteristics selected from age, sex and HLA type. The model may be a Bayesian model and the sequence data may have been obtained using whole genome sequencing. A Bayesian model may be implemented using a a gradient-based Markov chain Monte Carlo (MCMC) method to calculate the probability distributions for the segment usage of each V and J gene in the region of interest.

In general, a summarised value may be a median, average, trimmed average or any other known statistical metric of centrality of a plurality of values. A summarised read depth ratio value for a region may be a statistical metric of centrality for the plurality of read depth ratio values within the region. In embodiments where a model (such as e.g. a generalised additive model) is fitted to the read depth ratio profile, a summarised read depth ratio value for a region may be obtained as a statistical metric of centrality for the value of the fitted model within the region. For example, the average of the fitted model across the region may be used. In embodiments where a piecewise constant model is fitted to the read depth ratio profile, a summarised read depth ratio value for a region may be obtained as the value of the fitted model for the segment corresponding to the region or the plurality of segments corresponding to the region, or the value of the model for a segment within the region. For example, using a piecewise constant model constrained as described above, a summarised read depth ratio value for a subset of the region of interest that is likely to be deleted through VDJ recombination may be chosen as the minimum value of the model (which is also equal to the maximum deviation from 0 of the model).

When the fraction of lymphocytes is a fraction of B cells or T cells including any gene segment in the TCRA, TCRB, TCRG, TCRD, IGH, IGL or IGK gene locus, or a fraction of B cells or T cells including any gene segment listed in Table 8 or corresponding gene segments in another species, the subset of the region of interest that is likely to be deleted through VDJ recombination may correspond to the said gene segment. The summarised read depth ratio value for the gene segment may be a difference or absolute value of a difference between a summarised read depth ratio value for the gene segment and a summarised read depth ratio value for the preceding gene segment. The summarised read depth ratio values may be summarised log read depth ratios. The summarised read depth ratios may be obtained from the values of a model fitted to the read depth ratios. Any logs may be base 2 logarithms.

The method may further comprise obtaining a likelihood for the model fitted to the plurality of read depth ratios, wherein a likelihood below a predetermined threshold is indicative of excessive noise in the sample. The method may further comprise obtaining a plurality of candidate models and one or more statistical metrics (such as a likelihood and/or confidence interval) associated with the fitting of the plurality of candidate models, and selecting a candidate model based on the statistical metric(s). For example, a model with highest likelihood with a confidence interval within a predetermined range may be selected. Obtaining a plurality of candidate models may comprise obtaining a predetermined number of models, such as e.g. 10, 30, 50 or 100 models.

The method may further comprise obtaining a baseline read depth from a subset of the region of interest. The baseline read depth may be a summarised read depth value across the said subject of the region of interest. For example, the baseline read depth may be the median read depth across a predetermined subset of the region of interest. The subset of the region of interest used to obtain the baseline read depth may be a subset of the region of interest that is unlikely to be deleted through VDJ recombination. The subset of the region of interest that is unlikely to be deleted through VDJ recombination may comprise the n first V segments of the genomic locus that undergoes VDJ recombination. The subset of the region of interest that is unlikely to be deleted through VDJ recombination may comprise the C segment of the genomic locus that undergoes VDJ recombination. Preferably, the subset of the region of interest used to obtain the baseline read depth does not comprise any D or J segment of the genomic locus that undergoes VDJ recombination. Indeed, it is possible according to the methods disclosed herein to use the beginning, the end, or both the beginning and the end of the VDJ locus under investigation (instead of both), or other close regions that can be assumed to have copy number values unaffected by VDJ recombination The subset of the region of interest used to obtain the baseline read depth may be defined for respective VDJ loci using the genomic coordinates in Table 7, or corresponding coordinates for other species or reference genomes.

Without wishing to be bound by theory, it is believed that the use of longer normalisation regions (e.g. using both the beginning and end regions, and/or increasing lengths of the beginning region) advantageously compensates for noise that is typical in read depth data. For example, the beginning region can be extended to include a number of V segments that are unlikely to be frequently lost, or even regions outside of the gene if this data is available (such as e.g. when using whole genome sequencing data). Further, it is believed that the use of the signal at the beginning of the gene is beneficial because the signal associated with VDJ recombination is closer to the end of the gene than to its beginning (due to the J segments being clustered close to the end of the gene, whereas the V segments are more spread out). Additionally, it would also be possible to use a region outside of the VDJ locus under investigation, such as e.g. before or after the locus. However, increasing distances from the locus of interest increase the likelihood that the signal seen will correspond to a different copy number event in the cancer cells, and hence no longer reflect the tumour copy number at the VDJ locus under investigation. The parameter n may be determined using appropriate training data, for example by choosing the value of n that results in the most accurate estimate of lymphocyte fraction across the training data, when compared against an orthogonal metric of lymphocyte fraction. An orthogonal metric of lymphocyte fraction may be obtained for examples based on transcriptomic data and/or histopathology data. The parameter n may be between 8 and 16, between 10 and 14, such as e.g. 10. 11 or 12.

Obtaining a read depth profile may comprise obtaining a plurality of raw read depth values across the predetermined region of interest and smoothing the read depth values. Smoothing the read depth values may comprise replacing the raw read depth values by a corresponding rolling median over a window of a predetermined width. The predetermined width of the window may be selected between approximately 20 bp and approximately 200 bp, or between approximately 50 bp and approximately 150 bp. For example, the read depth profile may comprise values obtained by calculating a rolling median in 50 bp windows along the predetermined region of interest. Suitable sizes of windows can be determined empirically for a particular data set, type of data or context, by comparing the lymphocyte fraction estimates obtained using the present method with various candidate window sizes to orthogonal metrics of lymphocyte fractions such as the Danaher score.

The mixed sample may be a blood sample or a tumour sample. The method may further comprise obtaining a mixed sample comprising genomic material from multiple cell types, from a subject. The method may further comprise obtaining read depth data from a mixed sample comprising genomic material from multiple cell types, from a subject through one or more in vitro steps. The one or more in vitro steps may comprise nucleic acid extraction, library preparation, target sequence capture, sequencing and/or hybridisation to a copy number array. As the skilled person understands, once data indicative of the presence of nucleic acids in the sample has been obtained through e.g. sequencing, additional steps may be applied as known in the art or as described herein, such as e.g. quality control steps, reference genome alignment step (also referred to as mapping), normalisation steps, etc. The method may further comprise repeating the method of any embodiment of the present aspect for one or more further mixed sample(s), wherein the first and further mixed sample(s) have been obtained from the same subject and wherein the first and further mixed samples are tumour samples. The method may further comprise obtaining one or more summarised fraction(s) of lymphocytes based on the fraction of lymphocytes (f) for the first and further mixed samples. A summarised fraction of lymphocytes may be obtained the mean fraction across the first and further mixed samples, the minimum fraction across the first and further mixed samples, the maximum fraction across the first and further mixed samples, and/or the fold difference between the minimum fraction across the first and further mixed samples and the maximum fraction across the first and further mixed samples. Where a plurality of mixed samples are available for a subject, one or more of the summarised fraction(s) of lymphocytes may be used instead of the individual lymphocyte fraction for single samples to provide a prognosis as will be described further below. For example, the minimum fraction across the plurality of samples, the minimum and maximum fraction across the plurality of mixed samples, and/or the fold difference between the minimum fraction across the first and further mixed samples and the maximum fraction across the first and further mixed samples may be used to provide a prognosis for the subject. Preferably, the prognosis is provided using the minimum fraction across the plurality of samples.

The method may further comprise providing to a user, for example through a user interface, the determined lymphocyte fraction and/or any value derived therefrom. A value derived therefrom may comprise a diagnostic or prognostic indication.

As the skilled person understands, the complexity of the operations described herein (due at least to the amount of data that is typically generated by sequencing genomic DNA) are such that they are beyond the reach of a mental activity. Thus, unless context indicates otherwise (e.g. where sample preparation or acquisition steps are described), all steps of the methods described herein are computer implemented.

The methods of the present disclosure find use in multiple clinical contexts where different diagnosis and/or prognosis may be associated with different levels of lymphocytes in samples from subjects. Thus, the disclosure also relates to methods for stratifying a population of subjects according to their lymphocyte fraction status (e.g. tumour infiltrating lymphocyte-TIL-status), the method comprising carrying out the method of the first aspect of the disclosure on one or more samples (e.g. tumour samples) from each of the subjects in the population. For example, the subjects may be separated between an “immune cold” group and an “immune hot group”, depending on the lymphocyte fraction(s) estimated for the one or more tumour samples from the patent. This may finds particular application to the identification of patients for personalised medicine and/or clinical trials. Patients classified as immune cold are expected to be less responsive to immunotherapy such as using checkpoint inhibitors, compared to patients classified as immune hot. Thus, the latter may be more likely to benefit from immunotherapy, whereas the former may be more likely to benefit from alternative therapeutic options. Instead or in addition to this, patients classified may have poorer survival prognosis than patients classified as immune hot. Thus, the methods of the present disclosure also finds uses in classifying a subject that has been diagnosed with cancer between at least two groups that have different prognosis and/or predicted sensitivity to one or more immunotherapies.

Thus, according to a further aspect, the disclosure provides a method of providing a prognosis for a subject that has been diagnosed as having cancer, the method comprising determining the lymphocyte fraction in one or more tumour samples from the subject using the method of any embodiment of the first aspect. Also described is a method of monitoring a subject that has been diagnosed as having cancer, the method comprising determining the lymphocyte fraction in one or more tumour samples from the subject acquired at a first time point and in one or more tumour samples from the subject acquired at a further time point, using the method of any embodiment of the first aspect. For example, the first time point may be before treatment and the further time point may be after treatment. Alternatively, the first and further time points may both be after treatment. The treatment may be with any anti-cancer therapy, including immunotherapy. A method of providing a prognosis or monitoring a subject may further comprise classifying the sample(s) as immune hot or immune cold depending on the lymphocyte fraction (such as e.g. the T lymphocyte fraction) in the sample(s). For examples, samples with a lymphocyte fraction above a threshold (for example, about 0.1, i.e. about 10%) may be classified as immune hot, whereas samples with a lymphocyte fraction at or below the threshold may be classified as cold. The method may further comprise classifying the subject between a good prognosis group and a poor prognosis group depending on the number of samples from the subject that are classified as immune cold. For example, when analysing samples from different tumour regions, a number of samples classified as immune cold being at or above a threshold (such as e.g. 2) may classify the subject in the poor prognosis group. The poor prognosis group may be associated with reduced relapse free survival and/or reduced overall survival, compared to the good prognosis group. A threshold on the lymphocyte fraction and/or on the number of immune cold samples may be determined using an appropriate training cohort, for example by assessing the ability of various thresholds to classify patients between groups that are associated with significantly different prognosis (based on known response status). Also described is a method of determining whether a subject that has been diagnosed as having cancer is likely to respond to immunotherapy, the method comprising determining the lymphocyte fraction in one or more tumour samples from the subject using the method of any embodiment of the first aspect. The method may further comprise administering an immunotherapy, to a subject that has been diagnosed as likely to respond to immunotherapy. The method may comprise recommending a subject that has been diagnosed as likely to respond to an immunotherapy for treatment with the immunotherapy. The method may comprise administering an alternative therapy (e.g. a conventional chemotherapy, radiotherapy, etc.) and/or recommending a subject for treatment with an alternative therapy, where the subject has been diagnosed as not likely to respond to immunotherapy.

The method may further comprise classifying the subject between a group that is likely to respond to the immunotherapy, and a group that is not likely to respond to the immunotherapy. Indeed, the present inventors have demonstrated that the T cell fraction in tumour but also in blood samples, determined according to the present disclosure is predictive of response to immunotherapy. For example, the method may comprise classifying the sample(s) as immune hot or immune cold depending on the lymphocyte fraction (such as e.g. the T lymphocyte fraction) in the sample(s). For example, samples with a lymphocyte fraction above a threshold (for example, about 0.1, i.e. about 10%) may be classified as immune hot, whereas samples with a lymphocyte fraction at or below the threshold may be classified as cold. A subject may then be classified in the group that is not likely to respond to the immunotherapy if one or more samples from the subject are immune cold. Alternatively, a subject may be classified in the group that is not likely to respond to the immunotherapy if a sample from the subject has a lymphocyte fraction below a threshold, and in the group that is likely to respond to the immunotherapy otherwise.

The immunotherapy may be any therapy that modulates the function of the immune system and/or leverages an existing immune function to treat cancer. The immunotherapy may be selected from T-cell transfer therapy (tumour-infiltrating lymphocytes (or TIL) therapy and CAR T-cell therapy), therapeutic antibodies, cancer treatment vaccines, checkpoint inhibitor therapy, and immune system modulators (e.g. interferons and interleukins). The method may comprise classifying the subject between a group that is likely to respond to the immunotherapy (e.g. CPI therapy), and a group that is not likely to respond to the immunotherapy (e.g. CPI therapy), based on both the lymphocyte fraction in one or more samples from the subject, and an the Such estimated tumour mutational burden for subject. a classification may be obtained using a multivariate classification model (for example, a classification method using a trained generalised linear model). The parameters of a classification model (e.g. threshold(s), parameters of a multivariate model) may be determined using an appropriate training cohort, for example using a group of subjects with known Immunotherapy response status.

Also described is a method of determining whether a subject has T cell lymphopenia, the method comprising determining the lymphocyte fraction in one or more samples from the subject using the method of any embodiment of the first aspect. The one or more samples may be blood samples. The subject may be a neonate subject. The method of determining whether a subject has T cell lymphopenia may be performed in the context of a method for screening for severe combined immune deficiency. The method may further comprise treating the subject for lymphopenia, or recommending the subject for treatment for lymphopenia.

Also described is a method of diagnosing a subject as having a disease, disorder or condition associated with abnormal lymphocyte counts, the method comprising determining the lymphocyte fraction in one or more samples from the subject using the method of any embodiment of the first aspect. Also described is a method of monitoring a subject that has been diagnosed as having a disease, disorder or condition associated with abnormal lymphocyte counts, the method comprising determining the lymphocyte fraction in one or more samples from the subject using the method of any embodiment of the first aspect. The disease, disorder or condition associated with abnormal lymphocyte counts may be an autoimmune disease, a bone marrow disorder or an infection.

According to a further aspect, the present disclosure provides a system comprising:
at least one processor; and at least one non-transitory computer readable medium containing instructions that, when executed by the at least one processor, cause the at least one processor to perform operations comprising: obtaining a read depth profile for the sample comprising the read depths along a predetermined genomic region of interest, wherein the predetermined genomic region of interest includes at least a portion of a genomic locus that undergoes VDJ recombination; obtaining a plurality of read depth ratios (rᵢ) by normalising the read depths along the predetermined region of interest by reference to a baseline read depth derived from a subset of the region of interest; obtaining a summarised read depth ratio value (r_{VDJ}) for a subset of the region of interest that is likely to be deleted through VDJ recombination; and determining the fraction of lymphocytes (f) in the sample as a function of the summarised read depth ratio value (r_{VDJ}). The system according to the present aspect may have any of the features described in relation to the first aspect.

The system according to the present aspect may be configured to implement the method of any embodiment of the preceding aspects. In particular, the at least one non-transitory computer readable medium may contain instructions that, when executed by the at least one processor, cause the at least one processor to perform operations comprising any of the operations described in relation to the first aspect. The system may further comprise, in operable connection with the processor, one or more of: a user interface, wherein the instructions further cause the processor to provide, to the user interface for outputting to a user, at least the estimated value of the lymphocyte fraction or a value derived therefrom; one or more sequence read depth data acquisition device (such as e.g. a sequencing device); one or more data stores, such as e.g a sequence read depth data store.

According to a further aspect, there is provided a non-transitory computer readable medium or media comprising instructions that, when executed by at least one processor, cause the at least one processor to perform the method of any embodiment of any aspect described herein.

According to a further aspect, there is provided a computer program comprising code which, when the code is executed on a computer, causes the computer to perform the method of any embodiment of any aspect described herein.

The present invention includes the combination of the aspects and preferred features described except where such a combination is clearly impermissible or is stated to be expressly avoided. These and further aspects and embodiments of the invention are described in further detail below and with reference to the accompanying examples and figures.

### BRIEF DESCRIPTION OF THE FIGURES

FIG. 1 is a flowchart illustrating schematically a method of determining the lymphocyte fraction in a mixed sample.

FIG. 2 shows an embodiment of a system for determining the lymphocyte fraction in a mixed sample.

FIG. 3 illustrates schematically the biological concept behind the determination of the lymphocyte fraction in a mixed sample as described herein, using the VDJ recombination signal. A. Schematic demonstrating how read depth ratio signals are used to detect somatic copy number alteration gain or loss events in a standard tumour and matched germline analysis. In this analysis cells consist of three distinct cell types: tumour cells, T cells and all other stromal cells. B. Schematic of how this same process works when focusing on the TCRA gene in relation to VDJ recombination and removal of TRECs, the lower left panel indicates an increased number of breakpoints detected in the TRACERx100 dataset within the TCRA gene relative to surrounding areas of 14q suggesting that the TREC signal is captured.

FIG. 4A shows plots showing examples of read depth ratio in two TRACERx100 regions demonstrating either increased levels of T cell content in blood compared to matched tumour. FIG. 4B shows plots showing examples of read depth ratio in two TRACERx100 regions demonstrating increased levels of T cell content in tumour compared to matched blood. In FIG. 4A and FIG. 4B, VDV segments refer to variable segments in both the TCRα and TCRδ locus.

FIG. 5 shows schematically how the signal illustrated in FIG. 3 can be converted into a T cell fraction estimate according to embodiments of the disclosure.

FIG. 6A shows theoretical values for naïve TCRA T cell fraction scores for a range of local copy number and tumour purity values. Points and lines are coloured by the actual T cell fraction with the straight horizontal lines indicating where the points should be. FIG. 6B shows a distribution of local copy number of the TCRA gene across the TRACERx100 cohort. FIG. 6C shows a distribution of tumour purity across TRACERx100 cohort. FIG. 6D shows the exact vs naïve score by copy number for the TRACERx100 cohort, as can be seen in cases where copy number is 1 the naïve score is an overestimate and in other cases where it is above 2 it is an underestimate.

FIG. 7 shows the results of a validation of the approach described herein using cell line data. Pie charts of calculated TCRA T cell fraction from WES of either T cell derived cell lines or non-T cell derived cell lines are shown.

FIG. 8A shows the results of a validation of the approach described herein using simulated data in terms of calculated T cell fraction versus actual in simulated dataset with a background TCRA copy number of 2 and T cell fraction values ranging from 0.01 to 0.99. FIG. 8B shows the results of a validation of the approach described herein using simulated data in terms of simulated log read depth ratio from a sample consisting of 24% T cells and 75% tumour (TCRA copy number=1). FIG. 8C shows the results of a validation of the approach described herein using simulated data in terms of difference between calculated naïve T cell fraction and actual fraction for range of tumour copy number and purities. FIG. 8D shows the results of a validation of the approach described herein using simulated data in terms of difference between TCRA T cell fraction and actual fraction for a range of tumour copy number and purities.

FIG. 9A-D shows the results of a validation of the approach described herein by comparison with orthogonal metrics of immune infiltration in the TRACERx100 data. FIG. 9A shows association between estimated T cell fraction with Danaher RNA-based scores for CD8+, T cells and total TILs. FIG. 9B shows association with pathology TIL scores and measures of CD8+ T cell content either based on RNA-seq (Danaher, Davoli, EPIC, TIMER, CIBERSORT and xCell) or DNA (TIL ExTRECT and CDR3 VDJ score). FIG. 9C shows association between estimated TCRA T cell fraction with RNA-based scores for different immune related cell types from the Danaher, Davoli, EPIC, TIMER, CIBERSORT and xCell methods, scores are ordered by strength of association to the TCRA T cell fraction as assessed by the Spearman rho coefficient. FIG. 9D shows association of the CDR3 VDJ read score based on the iDNA method to TCRA T cell fraction.

FIG. 10A-B show the results of a validation of the approach described herein by comparison with orthogonal metrics of immune infiltrations. FIG. 10A shows lymphocyte fraction of TCGA LUAD and LUSC samples in the TRACERx100 data, calculated from methylation data versus TCRA T cell fractions. FIG. 10B shows lymphocyte fraction of TCGA LUAD and LUSC samples in the TRACERx100 data, calculated from methylation data versus CIBERSORT calculated CD8+ T cell fraction.

FIG. 11A-B shows the results of a validation of the approach described herein in terms of the effect of read coverage on the estimated TCRA T cell fraction. FIG. 11A shows results with down sampling of 5 TRACERx100 regions to different depths. FIG. 11B shows results with down sampling of simulated data to different depth levels. FIG. 11C shows the results of a validation of the approach described herein in terms of the effect of potential FFPE-related alterations on the estimated TCRA T cell fraction. The figure shows TCRA T cell fraction (non-GC corrected) value for FFPE and fresh frozen samples for bladder and melanoma tumours within the CPI1000+ cohort. FIG. 11D shows downsampling of the 5 TRACERx100 regions with the highest CDR3 read counts to different depths and the resulting CDR3 read counts.

FIG. 12A-C shows the results of the use of TCRA T cell fraction to obtain a prognosis indicator within the TRACERx100 cohort. FIG. 12A shows Kaplan-Meier curve showing survival difference between high and low risk patients as defined by the number of regions with <10% T cell content as inferred from TCRA T cell fraction estimation. FIG. 11B and FIG. 11C show Kaplan-Meier curves for LUAD the and LUSC histological subtypes in the TRACERx100.

FIG. 13A-C show the results of an analysis of TCRA T cell fraction within a TCGA cohort. FIG. 13A shows Kaplan-Meier curves showing overall survival in TCGA LUAD and LUSC. FIG. 12B shows Kaplan-Meier curves showing overall survival in LUAD alone. FIG. 12C shows Kaplan-Meier curves showing overall survival in LUSC alone with patients separated by the median TCRA T cell fraction within each group.

FIG. 14 shows the results of an analysis of TCRA T cell fraction within the TRACERx100 cohort. Boxplot showing the difference in TCRA T cell fraction estimation between blood and tumour in the TRACERx100.

FIG. 15 shows the results of an analysis of TCRA T cell fraction within the TRACERx100 cohort. Blood T cell fraction vs Tumour T cell fraction, divided by histology. Regions from the same patient are connected by lines where the type of line depends on whether all tumour regions have a T cell fraction that are higher (long dashes), lower (solid) or heterogeneous (short dashes) when compared to the matched blood T cell fraction in the TRACERx100.

FIG. 16A-F shows the results of an analysis of TCRA T cell fraction within the TRACERx100 cohort. FIG. 16A shows a boxplot showing the difference in TCRA T cell fraction between blood and tumour samples within the TRACERx100 cohort. FIG. 16B shows predictors of blood TCRA T cell fraction in TRACERx100 cohort. FIG. 16C shows Kaplan Meier curves for the multi-region TRACERx100 cohort separated by LUAD (top) and LUSC (bottom) divided by the number of cold regions present in the tumour (increasing left to right). Hot and cold regions were defined by using the mean of all the tumour regions (0.08095) as a threshold. In each Kaplan Meier curve the included patients were restricted to those with total regions greater than the number of cold regions used in defining the threshold. FIG. 16D shows hazard ratios of separate Cox regression models relating disease free survival to different multi-region measures related to the TCRA T cell fraction in the entire TRACERx100 cohort as well as the LUAD and LUSC patients separately. The relative region immune escape score is defined as the maximum region TCRA score divided by the minimum region (upper 95% CI) TCRA score. FIG. 16E shows a Cox proportional hazard model of the entire TRACERx100 cohort including the minimum and maximum regional TCRA T cell fraction, tumour stage, age and sex. FIG. 16F shows a Kaplan Meier curves for the multi-region TRACERx100 cohort separated by LUAD (top) and LUSC (bottom) divided by the number of cold regions present in the tumour (increasing left to right). Hot and cold regions were defined by using the median of all the tumour regions (0.0736) as a threshold. In each Kaplan Meier curve the included patients were restricted to those with total regions greater than the number of cold regions used in defining the threshold.

FIG. 17A-I shows the results of an analysis of TCRA T cell fraction within a TCGA cohort. FIG. 17A shows boxplots showing the difference in TCRA T cell fraction between blood and tumour samples for LUAD within the TCGA cohort. FIG. 187B shows boxplots showing the difference in TCRA T cell fraction between blood and tumour samples for LUSC within the TCGA cohort. FIG. 17C shows boxplots showing the difference in TCRA T cell fraction between LUAD and LUSC in blood samples within the TCGA cohort. FIG. 17D shows boxplots showing the difference in TCRA T cell fraction between LUAD and LUSC in tumour samples within the TCGA cohort. FIG. 17E shows predictors of blood TCRA T cell fraction in TCGA LUAD and LUSC cohort. FIG. 17F Kaplan Meier curves for overall and progression free survival in the TCGA LUAD cohort, dividing the cohort into immune hot and cold groups using the mean of the TCGA LUAD cohort (0.109) as a threshold. FIG. 17G shows hazard ratios of separate Cox regression models for TCRA T cell fraction for the TCGA LUAD and LUSC cohort for both overall survival (OS) and progression free survival (PFS). FIG. 17H shows Log 2 (Hazard ratios) from Kaplan Meier plots for the TCGA separating the tumour samples into hot and cold based on different thresholds from 0 to 0.16 in steps of 0.0025 for overall and progression free survival. FIG. 17I shows Kaplan Meier curves for the TCGA LUSC, and TCGA LUAD & LUSC cohorts for overall and progression free survival using the mean of the TCGA LUAD cohort (0.109) as a threshold for distinguishing hot and cold tumours.

FIG. 18A shows boxplots showing sex differences in TCRA T cell fraction in blood samples in the TRACERx100 cohort. FIG. 18B shows boxplots showing sex differences in TCRA T cell fraction in blood samples in the TCGA cohort. FIG. 18C shows the association between age and TCRA T cell fraction for germline blood samples in the TRACERx100 cohort. FIG. 18D shows the association between age and TCRA T cell fraction for germline blood samples in the TCGA cohort.

FIG. 19 shows a cohort overview of the CPI1000+ dataset.

FIG. 20 is a violin plot showing a significant difference between the calculated tumour TCRA T cell fraction for non-responders versus responders across the CPI1000+ meta cohort, dotted black line at 0.1 divides the tumours categorised as either immune hot or cold.

FIG. 21 is a plot showing tumour TCRA T cell fraction vs clonal TMB in the CPI1000+ data, dashed lines divide cohort into four quadrants with high/low mutational burden and hot/cold tumours. Inset pie charts indicate the percentage of patients that responded to CPI therapy.

FIG. 22 shows the results of a univariate meta-analysis of predictors of immune response across multiple cohorts containing at least 10 patients of an individual cancer type and both DNA and RNA-seq data. Left panel: forest plot showing the OR values of different clinical factors with associated p-values in terms of predictive value of response. Right panel: heatmap of OR values across individual studies within the CPI1000+ dataset, focusing on cohorts with both RNA-seq and TCRA T cell fractions available.

FIG. 23 shows ROC plot of GLM models for predicting CPI response in the CPI1000+ dataset (1: clonal TMB, 3: clonal TMB+TCRA T cell fraction, 2: clonal TMB+CD8A expression, 4: clonal TMB+TCRA T cell fraction+CD8A expression).

FIG. 24A shows a cohort overview of the CPI lung dataset, red lines in upper panel reflect the median TCRA T cell fraction in patients with (0.10) or without (0.0070) a response to CPI, note that Tumour TCRA T cell fraction is often zero. FIG. 24B shows the results of an univariate meta-analysis across three NSCLC CPI datasets with DNA sequencing but no RNA sequencing data.

FIG. 25 shows the TCRA T cell fraction for all regions in the TRACERx100 cohort depending on the number of TCRA V segments used in the read depth normalisation (see step 2 in FIG. 5) (top), the proportion of regions in the TRACERx100 cohort that are non-zero depending on the number of segments used (middle), and the −log (p-value) of the correlation with the Danaher T cell gene signature depending on the number of V segments used for normalisation (bottom).

FIG. 26A-D show an investigation of the effects of bias in the exon capture process used in the TCGA data. FIG. 26A is a plot showing an example of log read depth ratio for exons in a single sample from the TCGA LUAD cohort. Certain segments, particularly the J-gene exons, portray a bimodal distribution showing coverage biases. FIG. 26B shows median log ratio values for a selected range of exons in either the TRACERx100 (blue) or TCGA LUAD (red) cohort, with many exons in the TCGA LUAD cohort having much lower coverage and therefore log ratio values. FIG. 26C shows TCRA T cell fraction using the reduced exon set for the TCGA cohort before GC correction. FIG. 26D shows TCRA T cell fraction using the reduced exon set for the TCGA cohort after GC correction.

FIGS. 27A-D show an investigation of infection-related determinants of TCRA T cell fraction in blood and malignant tissue. FIGS. 27A-B shows association of microbial reads from KRAKEN with TCRA T cell fraction in blood and tumour samples. FIG. 27C shows microbial species for which normalised read count is significantly associated with TCRA T cell fraction in tumours in LUAD. FIG. 27D shows microbial species for which normalised read count is significantly associated with TCRA T cell fraction in tumours in LUSC (D).

FIG. 28A-H shows an investigation of determinants of TCRA T cell fraction in blood and malignant tissue with multi-region data. FIG. 29A shows an overview plot of PNE cohort containing multi-region microdisected tissue paired with normal blood samples. FIG. 28B-C show predictors of blood TCRA T cell fraction in PNE cohort. FIG. 28D shows the influence of tumour sample location on TCRA T cell fraction in the Messaoudene et al. breast cohort. FIG. 28E shows the influence of tumour sample location on TCRA T cell fraction in the TRACERx100 cohort (E). FIG. 28F shows a linear model for TCRA T cell fraction in PNE samples from genomic factors. FIG. 28G shows log 10 p-values for 59 microbial species tested for association with TCRA T cell fraction in blood and tumour sample in LUAD and LUSC. Red line represents the significance threshold at P=0.000423. FIG. 28H shows a summary of mean TCRA T cell fraction in PNE cohort.

FIGS. 29A-F shows an investigation of the association of subclonal SCNA with immune heterogeneity within a multiple-sample pan-cancer cohort. FIG. 29A shows an overview of immune heterogeneity across multi-sample pan-cancer cohort, dashed red line is at a TCRA T cell fraction of 0.1 FIG. 29B shows the proportion of patients in one of three categories, uniformly immune-hot (defined here as all regions ≥ 0.1 TCRA T cell fraction), uniformly immune-cold (defined here as all regions <0.1 TCRA T cell fraction) or heterogeneous with regions that are both hot and cold. FIG. 29C shows the subset of multi-sample patients (n=58) with heterogeneous immune infiltrate defined as having both a pair of regions with very similar TCRA T cell fraction (pairwise difference <0.1) and a pair of regions with very different TCRA T cell fraction (pairwise difference ≥0.1) plotted against pairwise SCNA heterogeneity defined as the combined proportion of the genome with SCNA changes unique to either region in the comparison. The data is averaged by tumour to control for any bias from tumours with large numbers of regions. FIG. 29D shows, on the lower panel: number of tumours in pan-cancer multi-sample cohort with subclonal gains (dark red-above 0 horizontal) or losses (dark blue-below 0) across the genome, horizontal lines signify the regions which have more than 30 tumours (see Methods) with subclonal gains or losses. FIG. 29D shows on the upper panel: −log 10 (p-value) of the 160 cytoband regions tested for association between TCRA T cell fraction and subclonal gains (dark red points) or losses (dark blue points). Red horizontal line marks significance threshold, only one region is significant, a loss event on chromosome 12q24.31-32. FIG. 29E shows TCRA T cell fraction change between regions with or without a subclonal loss event in chromosome 12q24.31-32. FIG. 29F shows a Volcano plot from Limma-Voom analysis of TRACERx100 tumours with a subclonal loss of 12q24.31-32 that was performed on regions with loss vs regions without loss. Only 8 genes were significant after multiple hypothesis adjustment, three of which (SPPL3, ABCB9 and OGFOD2) were in the loss region and are labelled.

FIG. 30 illustrates an implementation of the method of FIG. 5 for WGS data. Two alternatives are shown: a GAM model that uses a bin-based approach for GC correction and quality control, and a segment model that uses the known location of V and J segments. The segment model has the following constraints: 1. Model log read depth ratio as a linear model consisting of constant segments aligning to positions of V(D)J segments. 2. Model has value of 0 before first V segment. 3. V segments are monotonically decreasing in the order they appear in the genome e.g. TRVA2<TRVA1. 4. J segments are monotonically increasing in the order they appear in the genome. 5. Model has value of 0 after last segment.

FIGS. 31A-B show an example output of the GAM model of FIG. 30. FIG. 31A shows log 2read depth ratio along region encompassing positions 21600000-224000 and 14230000-14280000. FIG. 31B shows log 2read depth ratio along region encompassing positions 38240000-386000 and 10600000-10650000.

FIG. 32A-G show the results of Benchmarking of WGS T cell ExTRECT models using TRACERx100 cohort FIG. 32A shows a comparison of WGS and WES TCRA T cell fraction scores FIG. 32B shows the WGS T cell fraction scores compared to RNAseq Danaher scores for T cells FIG. 32C shows WGS B cell fraction scores compared to RNAseq Danaher scores for B cells, with entire TRACERx100 cohort. FIG. 32D shows WGS B cell fraction scores compared to RNAseq Danaher scores for B cells, for the TRACERx100 cohort with samples with low segment model log likelihood samples removed. FIG. 32E shows a comparison of T cell fraction scores from the WGS GAM model FIG. 32F shows a comparison of T cell fraction scores from the WGS segment model FIG. 32G shows a comparison of TRDV1 segment fraction from the segment model to transcripts per million (TPM) RNA scores from Kallisto for TCRD gene segments.

FIGS. 33A-J show the results of an analysis of the robustness of the methods of FIG. 30 with reduced sequencing depth. T cell ExTRECT scores were obtained for down-sampled bams using either the GAM or segment model. FIG. 33A shows results for the GAM model-TCRA. FIG. 33B shows results for the segment model-TCRA. FIG. 33C shows results for the gAM model-TCRB. FIG. 33D shows results for the segment model TCRB. FIG. 33E shows results for the GAM model-TCRG. FIG. 33G shows results for the GAM model-T cell average. FIG. 33I shows results for the GAM model-IGH. FIG. 33F shows results for the segment model-TCRG. FIG. 33H shows results for the segment model-T cell average. FIG. 33J shows results for the segment model-IGH.

FIGS. 34A-I show the use of the segment model on FIG. 30 to investigate TCR and BCR diversity. FIG. 34A-D show examples V segment usage plots for patient CRUK0085 (for TCRA, TCRB, and TCRG) and CRUK0045 (for IGH). FIG. 34E shows a heatmap of proportions of TRACERx100 samples utilising different TCRA V segments (segments list split over E-1 and E-2, legend on E-1). FIG. 34F-I show a comparison of number of V segments called with MixCR from matched RNAseq data with V segments called from the T cell ExTRECT segment model.

### DETAILED DESCRIPTION

In describing the present invention, the following terms will be employed, and are intended to be defined as indicated below.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

A “sample” as used herein may be a cell or tissue sample (e.g. a biopsy), a biological fluid, an extract (e.g. a DNA extract obtained from the subject), from which genomic material can be obtained for genomic analysis, such as genomic sequencing (whole genome sequencing, whole exome sequencing, targeted (also referred to as “panel” sequencing) or copy number array profiling. The sample may be a cell, tissue or biological fluid sample obtained from a subject (e.g. a biopsy). Such samples may be referred to as “subject samples”. In particular, the sample may be a blood sample, or a tumour sample, or a sample derived therefrom. The sample may be one which has been freshly obtained from a subject or may be one which has been processed and/or stored prior to genomic analysis (e.g. frozen, fixed or subjected to one or more purification, enrichment or extractions steps). In particular, the sample may be a cell or tissue culture sample. As such, a sample as described herein may refer to any type of sample comprising cells or genomic material derived therefrom, whether from a biological sample obtained from a subject, or from a sample obtained from e.g. a cell line. The sample is preferably from a jawed vertebrate (such as e.g. a jawed vertebrate cell sample or a sample from a jawed vertebrate subject), suitably from a mammalian (such as e.g. a mammalian cell sample or a sample from a mammalian subject, including in particular a model animal such as mouse, rat, etc.), preferably from a human (such as e.g. a human cell sample or a sample from a human subject). In embodiments, the sample is a sample obtained from a subject, such as a human subject. Further, the sample may be transported ad/or stored, and collection may take place at a location remote from the genomic sequence data acquisition (e.g. sequencing) location, and/or the computer-implemented method steps may take place at a location remote from the sample collection location and/or remote from the genomic data acquisition (e.g. sequencing) location (e.g. the computer-implemented method steps may be performed by means of a networked computer, such as by means of a “cloud” provider).

A “mixed sample” refers to a sample that is assumed to comprise multiple cell types or genetic material derived from multiple cell types. Samples obtained from subjects, such as e.g. tumour samples, are typically mixed samples (unless they are subject to one or more purification and/or separation steps). Preferably, a mixed sample is one that comprises lymphocytes, or is assumed to (expected to) to comprise lymphocytes. Suitably, the sample comprises lymphocytes and at least one other cell type. For example, the sample may be a tumour sample. A “tumour sample” refers to a sample derived from or obtained from a tumour. Such samples may comprise tumour cells, immune cells (such as e.g. lymphocytes), and other normal (non-tumour) cells. In the context of a tumour sample, the term “purity” refers to the proportion of cells in the sample that are tumour cells (also sometimes referred to as “cancer cell fraction” or “tumour fraction”), or to the equivalent proportion of cells in the case of a sample comprising genetic material derived from cells. In the context of samples comprising genetic material, a tumour fraction may be estimated using sequence analysis processes that attempt to deconvolute tumour and germline genomes such as e.g. ASCAT (Van Loo et al., 2010), ABSOLUTE (Carter et al., 2012), ichorCNA (Adalsteinsson et al., 2017), etc. In the context of tumour samples, the lymphocytes in the sample may be referred to as “tumour infiltrating lymphocytes” (TIL). A tumour sample may be a primary tumour sample, tumour-associated lymph node sample, or a sample from a metastatic site from the subject. A sample comprising tumour cells or genetic material derived from tumour cells may be a bodily fluid sample. Thus, the genetic material derived from tumour cells may be circulating tumour DNA or tumour DNA in exosomes. Instead or in addition to this, the sample may comprise circulating tumour cells. A mixed sample may be a sample of cells, tissue or bodily fluid that has been processed to extract genetic material. Methods for extracting genetic material from biological samples are known in the art. The lymphocytes may be T lymphocytes, B lymphocytes, or a mixture thereof. In particular, T lymphocytes may comprise αβ T lymphocytes and/or γδ lymphocytes. The B lymphocytes may comprise B cells with IGL light chains and/or B cells with IGK light chains.

The term “lymphocyte fraction” refers to the proportion of DNA containing cells within a mixed sample that are lymphocytes, or that are lymphocytes of a particular type (such as e.g. T lymphocytes, B lymphocytes, αβ T lymphocytes, T lymphocytes or B lymphocytes including any chosen gene segment in a TCR or BCR gene, etc.). The term “T lymphocyte fraction” refers to the proportion of DNA containing cells within a mixed sample that are T lymphocytes. The term “B lymphocyte fraction” refers to the proportion of DNA containing cells within a mixed sample that are B lymphocytes. Within the context of the present disclosure, a lymphocyte fraction is estimated based on a signal derived from a genomic region that undergoes VDJ recombination in the particular lymphocyte population that is being quantified. Thus, the lymphocyte fraction may refer more particularly to the proportion of DNA containing cells within a mixed sample that are lymphocytes characterised by having a predetermined genomic region of interest that has undergone VDJ recombination. The predetermined genomic region of interest may be the region of the TCRA/TCRD, TCRB, TCRG, IGH, IGL or IGK gene (also referred to herein as the TCRA/TCRD, TCRB, TCRG, IGH, IGL and IGK loci). Depending on the species under investigation, other less common loci may be used, such as the loci of specific TCRs present in marsupial (e.g. TCRμ) and shark. Depending on the choice of the predetermined region of interest (genomic locus) used in the present method, the lymphocyte fraction may capture a different subpopulation of lymphocytes. For example, using the TCRB locus may allow the quantification of the αβ T lymphocyte fraction. As another example, using the TCRA locus may allow the quantification of the combined αβ and γδ T lymphocyte fraction, as the TCRD locus is believed to be entirely contained locus. TCRA Further, using one or more segments within the corresponding to TCRD within the TCRA locus may allow the quantification of the fraction of αβ and γδ T lymphocytes that are γδ T lymphocytes. The choice of a particular segment corresponding to TCRD may be made depending on the correlation between the fraction estimated using the segment and an orthogonal metric indicative of the TCRD fraction. As another example, using the TCRG or the TCRD locus may allow the quantification of the γδ T lymphocyte fraction. Using the TCRG locus may be preferable for the quantification of the γδ T lymphocyte fraction as the TCRD locus is very small and hence would not be expected to contain as much informative signal. Conversely, using both the TCRA locus and the TCRB locus or the TCRG locus may enable the separate quantification of the αβ and γδ T lymphocyte fractions. As yet another example, using the IGH locus may allow the quantification of B cells (regardless of whether they have the IGL or IGK light chains), whereas using the IGL or IGK loci may allow the quantification of B cells that have either the IGL or the IGK light chains. Further, separate lymphocyte fractions may be quantified for multiple subpopulations of lymphocytes. These may be added to obtain a lymphocyte fraction that is representative of multiple or all subpopulations of lymphocytes. Additionally, some mixed samples may be assumed to primarily or exclusively contain a single population of lymphocytes. Thus, the lymphocyte fraction estimated using a single predetermined genomic region of interest may be assumed to be representative of the (overall) lymphocyte fraction for the sample. For example, a sample may be assumed to primarily contain T lymphocytes (or conversely, may be assumed to contain a relatively small proportion of B lymphocytes, compared to the proportion of T lymphocytes), the majority of which may be expected to be αβ T lymphocytes. Thus, in such cases a lymphocyte fraction estimated using a single predetermined genomic region of interest corresponding to the TCRA or TCRB loci. Similarly, some mixed samples may be assumed to primarily or exclusively contain a single population of T lymphocytes. Thus, the lymphocyte fraction estimated using a single predetermined genomic region of interest may be assumed to be representative of the (overall) T lymphocyte fraction for the sample. For example, αβ T lymphocytes may be assumed to be the primary type of T lymphocytes present in a sample, such that a T lymphocyte fraction may be estimated using a single predetermined genomic region of interest corresponding to the TCRA or TCRB loci. As another example, the TCRD locus is believed to be entirely contained within the TCRA locus (together forming a locus referred to as TCRA/TCRD), such that the lymphocyte fraction estimated using this single predetermined genomic region of interest may be assumed to be representative of the (overall) T lymphocyte fraction for the sample, including in particular αβ T lymphocytes and γδ T lymphocytes.

The TCRA gene refers to the T cell receptor alpha locus with Gene ID 6955 (HGNC symbol: TRA) in Homo Sapiens, or any homologous region in another jawed vertebrate. In Homo Sapiens, this gene is located on Chromosome 14, at location NC 000014.9 (21621904 . . . 22552132) (assembly GRCh38.p13), also referred to as location NC_000014.8 (22090057 . . . 23021075) (assembly GRCh37.p13). Examples of homologous loci include the mouse tora locus (gene ID: 21473, located on Chromosome 14, assembly GRCm39-NC 000080.7 (52665424 . . . 54461655), assembly GRCm38.p6-NC_000080.6 (52427967 . . . 54224198)). Examples of coordinates that can be used as the region of maximum V(D)J recombination and as regions used to obtain a baseline read depth for the TCRA gene (and any gene therein such as the TCRD gene) are provided in Table 7.

The TCRB gene refers to the T cell receptor beta locus with Gene ID 6957 (HGNC symbol: TRB) in Homo Sapiens, or any homologous region in another jawed vertebrate. In Homo Sapiens, this gene is located on Chromosome 7, at location NC_000007.14 (142299011 . . . 142813287) (assembly GRCh38.p13) or NC_000007.13 (141998851 . . . 142510972) (assembly GRCh37.p13). Examples of homologous loci include the mouse tcrb locus (Gene ID: 21577), located on chromosome 6, assembly GRCm39-NC_000072.7 (40868230 . . . 41535305), or assembly GRCm38.p6-NC_000072.6 (40891296 . . . 41558371). Examples of coordinates that can be used as the region of maximum V(D)J recombination and as regions used to obtain a baseline read depth for the TCRB gene are provided in Table 7.

The TCRG gene refers to the T cell receptor gamma locus with Gene ID 6965 (HGNC symbol: TRG) in Homo Sapiens, or any homologous region in another jawed vertebrate. In Homo Sapiens, this gene is located on Chromosome 7, at location NC_000007.14 (38240024 . . . 38368055, complement) (GRCh38.p13) or NC_000007.13 (38279625 . . . 38407656, complement) (GRCh37.p13). Examples of homologous loci include the mouse torg locus (gene ID: 110067, located on Chromosome 13, assembly GRCm39-NC_000079.7 (19362212 . . . 19540646), assembly GRCm38.p6-NC_000079.6 (19178042 . . . 19356476)). Examples of coordinates that can be used as the region of maximum V(D)J recombination and as regions used to obtain a baseline read depth for the TCRG gene are provided in Table 7.

The TCRD gene refers to the T cell receptor delta locus with Gene ID 6964 (HGNC symbol: TRD) in Homo Sapiens, or any homologous region in another jawed vertebrate. In Homo Sapiens, this gene is located on Chromosome 14, at location NC_000014.9 (22422546 . . . 22466577) (GRCh38.p13) or NC_000014.8 (22891537 . . . 22935569) (GRCh37.p13). Examples of homologous loci include the mouse tord locus (gene ID: 110066, located on Chromosome 14, assembly GRCm39-NC_000080.7 (54183530 . . . 54396655), assembly GRCm38.p6 NC_000080.6 (53946073 . . . 54159198)). Examples of coordinates that can be used as the region of maximum V(D)J recombination and as regions used to obtain a baseline read depth for the TCRD gene are provided in Table 7 (by reference to the regions used in TCRA) and Table 8 (by reference to the TCRD segments within the TCRA locus).

The IGH gene refers to the immunoglobulin heavy locus with Gene ID 3492 (HGNC symbol: IGH) in Homo Sapiens, or any homologous region in another jawed vertebrate. In Homo Sapiens, this gene is located on Chromosome 14, at location NC_000014.9 (105586437 . . . 106879844, complement) (GRCh38.p13) or NC_000014.8 (106032614 . . . 107288051, complement) (GRCh37.p13). Examples of homologous loci include the mouse Igh locus (gene ID: 11507, located on Chromosome 12, assembly GRCm39-NC_000078.7 (113222388 . . . 115973574, complement), assembly GRCm38.p6-NC_000078.6 (113258768 . . . 116009954, complement)). Examples of coordinates that can be used as the region of maximum V(D)J recombination and as regions used to obtain a baseline read depth for the IGH gene are provided in Table 7.

The IGL gene refers to the immunoglobulin lambda locus with Gene ID 3535 (HGNC symbol: IGL) in Homo Sapiens, or any homologous region in another jawed vertebrate. In Homo Sapiens, this gene is located on Chromosome 22, at location NC_000022.11 (22026076 . . . 22922913) (GRCh38.p13) or NC_000022.10 (22380474 . . . 23265085) (GRCh37.p13). Examples of homologous loci include the mouse Ig1 locus (gene ID: 111519, located on Chromosome 16, assembly GRCm39-NC_000082.7 (18845608 . . . 19079594, complement), assembly GRCm38.p6-NC_000082.6 (19026858 . . . 19260844, complement)).

The IGK gene refers to the immunoglobulin kappa locus with Gene ID 50802 (HGNC symbol: IGK) in Homo Sapiens, or any homologous region in another jawed vertebrate. In Homo Sapiens, this gene is located on Chromosome 2, location at NC_000002.12 (88857361 . . . 90235368) (GRCh38.p13) or NC_000002.11 (89890568 . . . 90274235) 1 (89156874 . . . 89630436, complement) (GRCh37.p13). Examples of homologous loci include the mouse Igk locus (gene ID: 243469, located on Chromosome 6, assembly GRCm39-NC_000072.7 (67532620 . . . 70703738), assembly GRCm38.p6-NC_000072.6 (67555636 . . . 70726754)).

The term “sequence data” refers to information that is indicative of the presence and preferably also the amount of genomic material in a sample that has a particular sequence. Such information may be obtained using sequencing technologies, such as e.g. next generation sequencing (NGS, such as e.g. whole exome sequencing (WES), whole genome sequencing (WGS), or sequencing of captured genomic loci (targeted or panel sequencing)), or using array technologies, such as e.g. copy number variation arrays, or other molecular counting assays. When NGS technologies are used, the sequence data may comprise a count of the number of sequencing reads that have a particular sequence. Sequence data may be mapped to a reference sequence, for example a reference genome, using methods known in the art (such as e.g. Bowtie (Langmead et al., 2009)). Thus, counts of sequencing reads or equivalent non-digital signals may be associated with a particular genomic location (where the “genomic location” refers to a location in the reference genome to which the sequence data was mapped). The term “read depth” refers to a signal that is indicative of the amount of genomic material in a sample that maps to a particular genomic location. Such a signal may be obtained using sequencing technologies, such as e.g. next generation sequencing (NGS, such as e.g. WES, WGS, or sequencing of captured genomic loci), or using array technologies, such as e.g. copy number variation arrays. When NGS technologies are used, a read depth may be a read depth within the common sense of the word, i.e. a count of the number of sequencing reads mapping to a genomic location. When array technologies are used, a read depth may be an intensity value associated with a particular genomic location, which can be compared to a control to provide an indication of the amount of genomic material that maps to the particular location. The term “read depth profile” refers to a collection of read depth values relating to a plurality of genomic locations. For example, a read depth at a particular genomic location i may refer to the read depth at the base at position i in a reference genome, and a read depth profile may refer to the read depth for a plurality of positions i within one or more regions of interest.

As used herein “treatment” refers to reducing, alleviating or eliminating one or more symptoms of the disease which is being treated, relative to the symptoms prior to treatment. “Prevention” (or prophylaxis) refers to delaying or preventing the onset of the symptoms of the disease. Prevention may be absolute (such that no disease occurs) or may be effective only in some individuals or for a limited amount of time.

As used herein, the terms “computer system” includes the hardware, software and data storage devices for embodying a system or carrying out a method according to the above described embodiments. For example, a computer system may comprise a processing unit (such as a central processing unit (CPU) and/or graphical processing unit (GPU)), input means, output means and data storage, which may be embodied as one or more connected computing devices. Preferably the computer system has a display or comprises a computing device that has a display to provide a visual output display (for example in the design of the business process). The data storage may comprise RAM, disk drives or other computer readable media. The computer system may include a plurality of computing devices connected by a network and able to communicate with each other over that network. It is explicitly envisaged that computer system may consist of or comprise a cloud computer.

As used herein, the term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

### Determining the Lymphocyte Fraction in a Mixed Sample

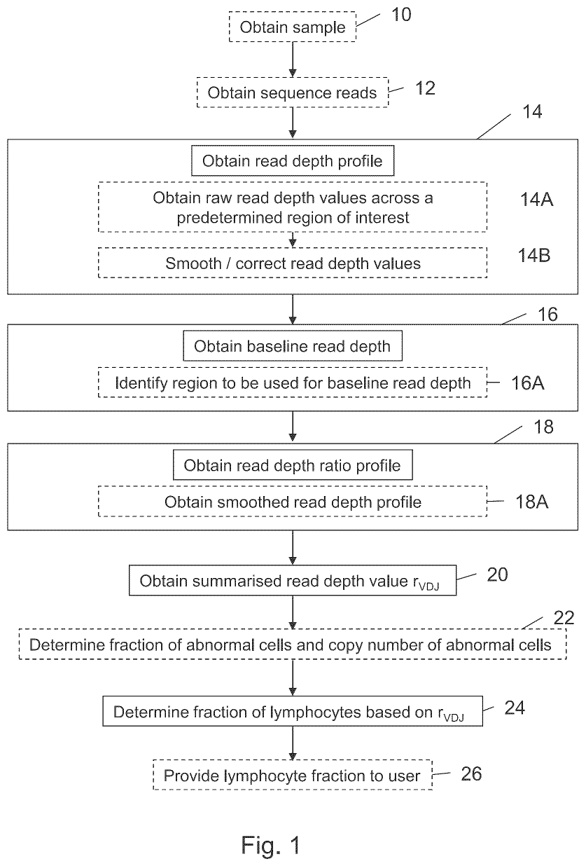

The present disclosure provides method for determining the lymphocyte fraction in a mixed sample, using read depth data from the sample comprising read depth data for at least a predetermined genomic region of interest. An illustrative method will be described by reference to FIG. 1. At optional step 10, a mixed sample comprising genomic material from multiple cell types may be obtained from a subject. At optional step 12, sequence reads/read depth data may be obtained from the mixed sample, for example by sequencing the genomic material in the sample using one of whole exome sequencing, whole genome sequencing, or panel sequencing. At step 14, a read depth profile for the sample comprising the read depths along a predetermined genomic region of interest is obtained. This may comprise step 14A of obtaining a plurality of raw read depth values across the predetermined region of interest and step 14B of smoothing and/or correcting (e.g. GC correction) the read depth values.

A step 16, a baseline read depth is obtained from a subset of the region of interest. This may comprise optional step 16A of identifying a subset of the region of interest that can be used to obtain the baseline read depth as a subset of the region of interest that is unlikely to be deleted through VDJ recombination. For example, the region of interest that is unlikely to be deleted through VDJ recombination comprise the n first V segments of the genomic locus that undergoes VDJ recombination. The parameter n may be determined using appropriate training data, for example by choosing the value of n that results in the most accurate estimate of lymphocyte fraction across the training data, when compared against an orthogonal metric of lymphocyte fraction. An orthogonal metric of lymphocyte fraction may be obtained for examples based on transcriptomic data and/or histopathology data.

At step 18, a plurality of read depth ratios (rᵢ) are obtained by normalising the read depths along the predetermined region of interest by reference to the baseline read depth obtained at step 16. This may comprise optional step 18A of fitting a model to the read depth ratio profile (comprising the plurality of read depth ratios (rᵢ)) to obtain a smoothed read depth profile. At step 20, a summarised read depth ratio value (r_{VDJ}) is obtained for a subset of the region of interest that is likely to be deleted through VDJ recombination (optionally based on the value of the model fitted at step 18A, in the subset of the region of interest that is likely to be deleted through VDJ recombination).

At step 22, the fraction of abnormal cells (p) in the mixed sample, where abnormal cells are those that are aneuploid in the predetermined region of interest, and the copy number of the abnormal cells in the predetermined region of interest (Vr) may optionally be determined by obtaining an abnormal cell-specific copy number profile using methods known in the art. At step 24, the fraction of lymphocytes (f) in the sample is determined as a function of the summarised read depth ratio value (r_{VDJ}) obtained at step 20. This may comprise determining the fraction of lymphocytes (f) in the sample as a function of the summarised read depth ratio value (r_{VDJ}), the fraction of abnormal cells (p) in the mixed sample and the copy number of the abnormal cells in the predetermined region of interest (Vr).

At optional step 26, the determined lymphocyte fraction and/or any value derived therefrom may be provided to a user, for example through a user interface. The value derived therefrom may include prognostic and/or diagnostic information, as described further below.

### Applications

The above methods find applications in a variety of clinical contexts. For example, in the context of cancer, determining the lymphocyte fraction in a tumour sample, or preferably in a plurality of samples from a tumour, may provide an indication of the immune status of the tumour. This has been shown to have prognostic value for a variety of cancers, including e.g. non small cell lung (as demonstrated herein), ovarian cancer (see e.g. Zhang et al., 2003), colorectal cancer (see e.g. Galon et al., 2006), breast cancer (see e.g. Dieci et al., 2018) and melanoma. The present inventors have demonstrated that the T and/or B cell fraction in blood and/or tumour samples is indicative of prognostic in at least some cancer types (see Example 6, FIG. 35).

Thus, also described herein are methods of the immune status of a tumour from a subject, the method comprising determining the lymphocyte fraction in one or more tumour samples from the subject.

Additionally, also described herein are methods of providing a prognosis for a subject that has been diagnosed as having a cancer, the method comprising determining the lymphocyte fraction in one or more tumour samples from the subject. The method may further comprise classifying the sample(s) as immune hot or immune cold depending on the lymphocyte fraction (such as e.g. the T lymphocyte fraction) in the sample(s). For examples, samples with a lymphocyte fraction above a threshold (for example, about 0.1, i.e. about 10%) may be classified as immune hot, whereas samples with a lymphocyte fraction at or below the threshold may be classified as cold. The method may further comprise classifying the subject between a good prognosis group and a poor prognosis group depending on the number of samples from the patient that are classified as immune cold. For example, when analysing samples from different tumour regions, a number of samples classified as immune cold being at or above a threshold (such as e.g. 2) may classify the subject in the poor prognosis group. The poor prognosis group may be associated with reduced relapse free survival and/or reduced overall survival, compared to the good prognosis group. A threshold on the lymphocyte fraction and/or on the number of immune cold samples may be determined using an appropriate training cohort, for example by assessing the ability of various thresholds to classify patients between groups that are associated with significantly different prognosis.

Additionally, still in the context of cancer, the presence of TILs has been shown to be associated with an increased likelihood of response to immunotherapy, including in particular checkpoint inhibitors (CPI) (see e.g. Litchfield et al., 2020), but also any other forms of immunotherapy that leverage an existing immune function such as T-cell transfer therapy (tumour-infiltrating lymphocytes (or TIL) therapy and CAR T-cell therapy), therapeutic antibodies, cancer treatment vaccines and immune system modulators (e.g. interferons and interleukins). Thus, also described herein are methods of determining whether a subject that has been diagnosed as having a cancer is likely to benefit from treatment with an immunotherapy, the method comprising determining the lymphocyte fraction in one or more tumour samples from the subject. The immunotherapy may be any therapy that modulates the function of the immune system to treat cancer. Indeed, any such therapy is believed to be likely to be influenced by the presence or absence of immune cells within the tumour. In embodiments, the immunotherapy is a CPI therapy. CPI therapy includes for example treatment with an anti-CTL4 or anti-PDL1 drug. The method may further comprise classifying the subject between a group that is likely to respond to the immunotherapy therapy, and a group that is not likely to respond to the immunotherapy. For example, the method may comprise classifying the sample(s) as immune hot or immune cold depending on the lymphocyte fraction (such as e.g. the T lymphocyte fraction) in the sample(s) (as explained above). A subject may then be classified in the group that is not likely to respond to the immunotherapy (e.g. CPI therapy) if one or more samples from the subject are immune cold. Alternatively, a subject may be classified in the group that is not likely to respond to the immunotherapy (e.g. CPI therapy) if a sample from the subject has a lymphocyte fraction below a threshold, and in the group that is likely to respond to the immunotherapy (e.g. CPI therapy) otherwise. Alternatively, the method may further comprise classifying the subject between a group that is likely to respond to CPI therapy, and a group that is not likely to respond to CPI therapy, based on both the lymphocyte fraction in one or more samples from the subject, and the estimated tumour mutational burden for the a subject. Such classification may be obtained using a multivariate model. The parameters of a classification model (e.g. threshold(s), parameters of a multivariate model) may be determined using an appropriate training cohort.

The lymphocyte fraction as described herein may also be used to determine whether a subject has severe combined immune deficiency (SCID), and/or T cell lymphopenia. This finds particular use in the context of neonatal care. Indeed, prior to the present method, SCID was typically identified using a complete blood cell count. This could in theory be confirmed using sequencing and quantification of the TRECs themselves but this is rarely done in a real clinical setting. The ability to provide a diagnostic for SCID using data from WES, which is increasingly performed as part of genomic screening of neonates to identify potentially damaging germline mutations, is therefore very valuable. Thus, the disclosure also relates to a method of identifying T cell lymphopenia and/or SCID in a subject, particularly a neonate subject, the method comprising determining the lymphocyte fraction as described herein, in one or more samples from the subject. The sample(s) may for example be blood sample(s).

The present methods also find uses in any clinical context where the presence/absence or abundance of immune cells or specific types of immune cells in a sample is indicative of a diagnosis or prognosis. For example, some autoimmune diseases are associated with low lymphocyte counts (e.g. lupus, rheumatoid arthritis), as are some bone marrow disorders (e.g. aplastic anaemia) and infections (such as HIV, sepsis, influenza, malaria, viral hepatitis, tuberculosis, typhoid fever, etc.). Thus, also described herein are methods of diagnosing a subject as having a disease, disorder or condition associated with abnormal lymphocyte counts, the method comprising determining the lymphocyte fraction in one or more samples from the subject.

The lymphocyte fraction as described herein may also be used to determine whether one or more germline mutations such as single nucleotide polymorphisms (SNPs) are associated with increased or reduced T/B cell fraction, for example in blood samples, e.g. through a GWAS analysis. This may help to identify variants (germline mutations) that may be causative of the increased/reduced T cell fraction.

### Systems

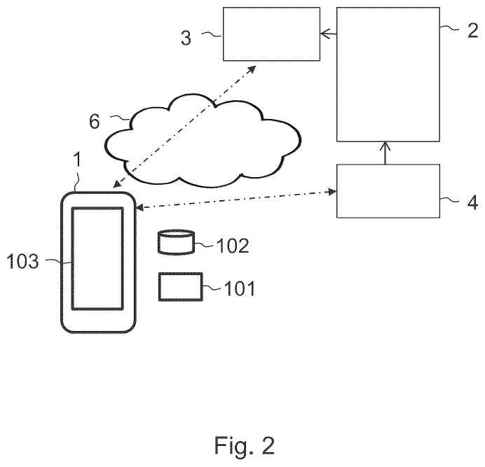

FIG. 2 shows an embodiment of a system for determining the lymphocyte fraction in a mixed sample and/or for providing a prognosis or treatment recommendation based at least in part on the lymphocyte fraction, according to the present disclosure. The system comprises a computing device 1, which comprises a processor 101 and computer readable memory 102. In the embodiment shown, the computing device 1 also comprises a user interface 103, which is illustrated as a screen but may include any other means of conveying information to a user such as e.g. through audible or visual signals. The computing device 1 is communicably connected, such as e.g. through a network 6, to read depth data acquisition means 3, such as a sequencing machine, and/or to one or more databases 2 storing read depth data. The one or more databases may additionally store other types of information that may be used by the computing device 1, such as e.g. reference sequences, parameters, etc. The computing device may be a smartphone, tablet, personal computer or other computing device. The computing device is configured to implement a method for determining the lymphocyte fraction in a mixed sample, as described herein. In alternative embodiments, the computing device 1 is configured to communicate with a remote computing device (not shown), which is itself configured to implement a method of determining the lymphocyte fraction in a mixed sample, as described herein. In such cases, the remote computing device may also be configured to send the result of the method of determining the lymphocyte fraction to the computing device. Communication between the computing device 1 and the remote computing device may be through a wired or wireless connection, and may occur over a local or public network such as e.g. over the public internet or over Wifi. The read depth data acquisition means may be in wired connection with the computing device 1, or may be able to communicate through a wireless connection, such as e.g. through WiFi, as illustrated. The connection between the computing device 1 and the read depth data acquisition means 3 may be direct or indirect (such as e.g. through a remote computer). The read depth data acquisition means 3 are configured to acquire read depth data from nucleic acid samples, for example genomic DNA samples extracted from cells and/or tissue samples. In some embodiments, the sample may have been subject to one or more preprocessing steps such as DNA purification, fragmentation, library preparation, target sequence capture (such as e.g. exon capture and/or panel sequence capture). Preferably, the sample has not been subject to amplification, or when it has been subject to amplification this was done in the presence of amplification bias controlling means such as e.g. using unique molecular identifiers. Any sample preparation process that is suitable for use in the determination of a genomic copy number profile (whether whole genome or sequence specific) may be used within the context of the present disclosure. The read depth data acquisition means is preferably a next generation sequencer. The sequence data acquisition means 3 may be in direct or indirect connection with one or more databases 2, on which sequence data (raw or partially processed) may be stored.

The following is presented by way of example and is not to be construed as a limitation to the scope of the claims.

### EXAMPLES

The immune microenvironment influences tumour evolution and can be both prognostic and predictive for response to immunotherapy. However, measuring tumour infiltrating lymphocytes (TILs) is restricted by lack of appropriate data. Whole exome sequencing (WES) of DNA is frequently performed to calculate tumour mutational burden and identify actionable mutations. In these examples, the inventors develop a method for T cell fraction estimation from WES samples, using a signal from T cell receptor excision circles (TRECs) loss during VDJ recombination of the T cell receptor alpha (TCRA) gene. The method is applicable to various types of clinically relevant samples including cancer samples, e.g. to measure TIL. In these examples, the inventors show that this score significantly correlates with orthogonal TIL estimates and can be calculated from fresh frozen or formalin-fixed paraffin-embedded samples. Blood TCRA T cell fraction was correlated with immune infiltrate in tumours, presence of bacterial sequencing reads and was higher in females. Tumour TCRA T cell fraction was prognostic in lung adenocarcinomas and using a meta-analysis of immunotherapy treated tumours, revealed that this score was predictive of response to immunotherapy, providing value beyond mutational burden. Applying this score to a multi-sample pan-cancer cohort revealed extensive diversity in immune infiltration within tumours. Subclonal loss of 12q24.31-32, encompassing SPPL3, was associated with reduced TCRA T-cell fraction. The method described herein, T cell ExTRECT (T cell Exome TREC Tool), elucidates the T cell infiltrate of WES samples.

### Example 1—Estimation of T Cell Fraction in a Sample from WES Data

### Introduction

Determining the level of TILs within a tumour is of key importance to both understand the tumour immune microenvironment and predict response to immunotherapy. However, currently there is no universal state-of-the-art method for estimating TILs. Microarray signatures such as CIBERSORT (Newman et al., 2015) as well as RNA-seq signatures such as CIBERSORTX for RNA-seq (Newman et al., 2019), ESTIMATE (Yoshihara et al., 2013) and those compiled by Danaher et al., have been used to quantify the transcriptomic signatures of TILs in tumour samples. Alternatively, the presence of immune infiltrate can be determined based on haematoxylin and eosin (H&E) histopathological slides or alternatively with the appropriate cell-specific staining, most commonly immunohistochemistry. If more phenotypic knowledge of the T cells is required, T cell receptor (TCR) sequencing can be performed following enrichment for those sequences encoding the highly diverse VDJ recombined CDR3 region of the TCR (Bolotin et al., 2012).

A significant drawback of most existing methods of T cell quantification is that they require additional material, time and expertise, beyond WES. Thus, while these methods provide additional knowledge that could aid the prediction of immune response, the application of these methods in a clinical setting requires increased overhead of both time and expenditure, adding to the already restrictive cost of immunotherapy.

In this example, the inventors propose a method for the estimation of T cell fraction within a sample directly from WES data. This method utilises a somatic copy number-based signal from VDJ recombination and the excision of T cell receptor excision circles (TRECs).

The method has the potential to be calculated from any form of NGS platform including both whole genome sequencing and targeted panel-based methods, as well as being extended to all genes undergoing VDJ recombination.

### Results

T cell diversity is a product of VDJ recombination, where the different gene segments within the T cell receptor gene recombine. The result of this is the excision of a large number of unselected gene segments from the TCRA gene as TRECs. This process leads to a copy number difference between T cells and other cells, with the T cell effectively undergoing a deletion event within the TCRA gene.

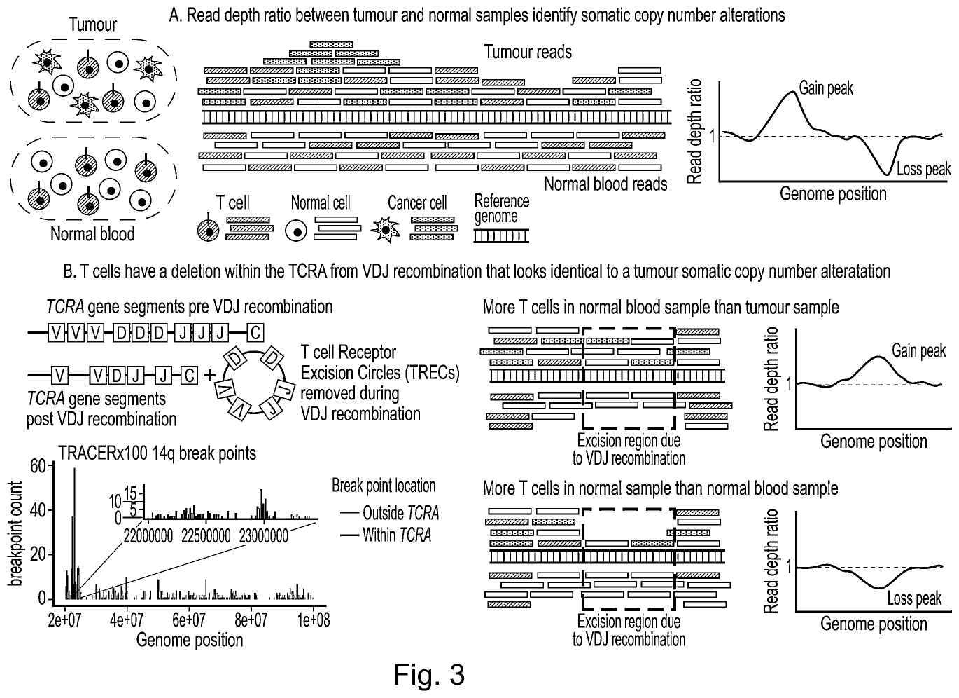

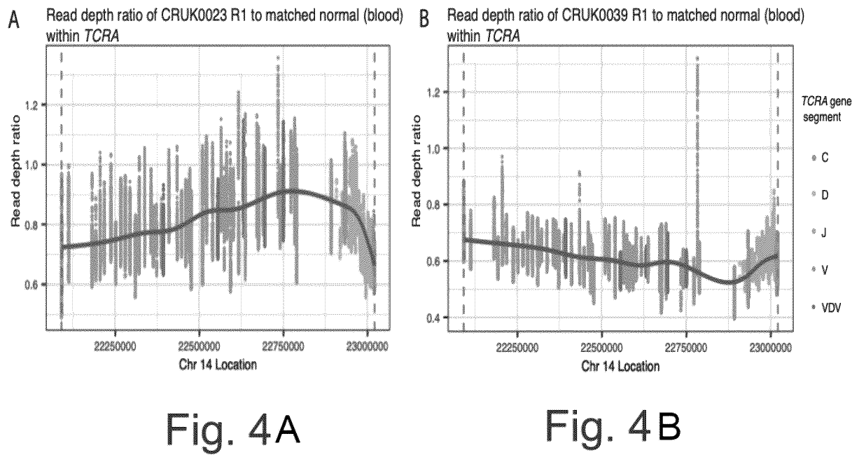

Standard somatic copy number alteration estimation tools (e.g. ASCAT (Van Loo et al., 2010), Sequenza (Favero et al., 2015), FACETS (Shen & Seshan, 2015), or ABSOLUTE (Carter et al., 2012)) principally rely on two related signals to obtain a cancer somatic copy number alteration profile; the B-allele frequency, reflecting the relative frequency of heterozygous SNPs in the tumour sample and the read depth ratio, reflecting the log of the ratio of reads between the tumour sample and its matched germline (often the buffy coat from a blood sample). Any deviance in the read depth ratio is assumed to specifically reflect a change in copy number in the tumour (as illustrated on FIG. 3A). However, in the context of the TCRA gene, this assumption does not hold; instead of solely reflecting a somatic copy number alteration in the tumour, a deviance in the read depth ratio overlapping the TCRA gene will also reflect the detection of deletion events within the TCRA gene of T cells also present in the tumour sample. Whether this is a loss or gain event depends on the difference in the proportion of T cells present in the blood compared to the tumour sample (as illustrated on FIG. 3B). Specifically, if the T cell content in the sequenced tumour sample is lower compared to the blood sample, there will be more reads from the TCRA gene identified in the tumour sample, leading to an amplification signal (log read depth ratio >0). Conversely, if there is either a higher T cell content in the tumour compared to the blood there will be fewer TCRA reads and the somatic copy number alteration estimation tool will infer a deletion or loss event (read depth ratio <0).

Upon examining the ASCAT derived somatic copy number alteration profiles of the TRACERx100 cohort (Jamal-Hanjani et al., 2017) of non-small cell 1 lung cancer (NSCLC) tumours subjected to multi-region sequencing, the inventors noted that somatic copy number alteration segments with both breakpoints being within the TCRA gene occurred in 165/327 of the tumour regions subject to WES (as illustrated on FIG. 1B). Examining two such cases in the TRACERx100 cohort, the read depth ratio was found to form a peak at the genomic location of the D genomic segments, which is the location most frequently included within TRECs (as illustrated on FIG. 4). These data therefore suggest that a signal of TRECs can be identified from WES data.

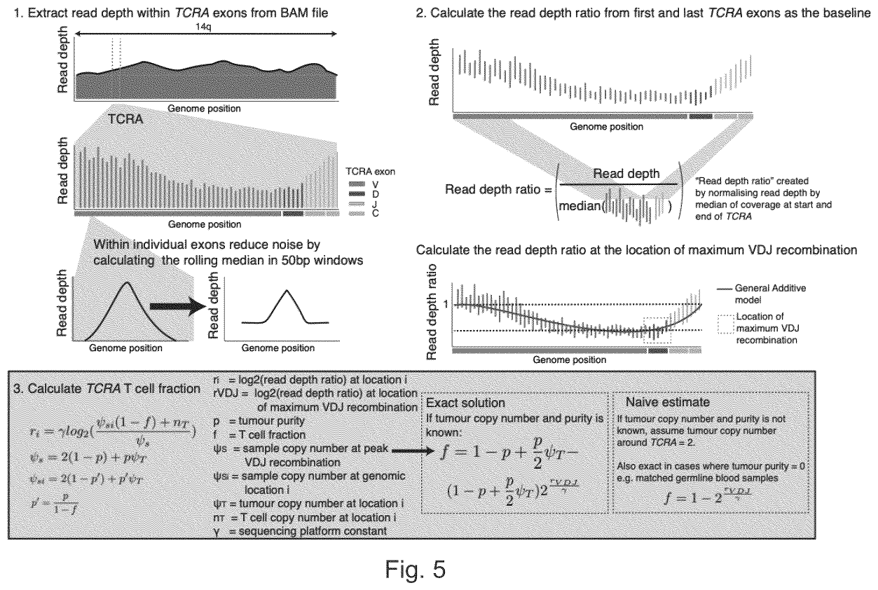

To explicitly exploit this signal to calculate a T cell fraction within individual samples, the inventors developed: TIL ExTRECT (T cell Exome T-cell Receptor Excision Circle Tool). In brief, TIL ExTRECT relies on an analysis of the read depth ratio within the TCRA gene to directly measure T cell infiltrate in WES samples. First, the read depth at each position within the TCRA gene is calculated, using the genomic segments at the beginning and end of TCRA that are unaffected by VDJ recombination as a control, and a modified log read depth ratio is calculated between the original read depth of the coverage values and the median of the values within the control genomic segments. Finally, provided both the fraction of tumour cells within the sequenced sample (the tumour purity) and the local somatic copy number around TCRA is known, an exact estimation of the T cell fraction can be calculated based on the size of the deviation of the log (base 2) read depth ratio. If knowledge of the tumour purity and local somatic copy number is unavailable, a naïve estimation can also be made which we demonstrate to have a similar predictive value (as explained in detail in the methods section in as illustrated on FIG. 5). Notably, unlike RNA-seq scores, the TCRA score represents a direct measurement of the proportion of T cells. This is referred to from here as the “TCRA T cell fraction”.

A full description of TIL ExTRECT along with the optimisation of its parameters such as the choice of segments for normalisation and quality control of the exons selected is presented in the Methods below. The accuracy of the TCRA T cell fraction was validated using four orthogonal approaches described in Example 2 below. Then, its clinical utility was demonstrated using two separate approaches, in Examples 3 and 4. Finally, its use in evaluating the key determinants of T cell immune infiltrate in different sample types was investigated in Example 5.

### Methods

Definition of Local Copy Number Values Around the TCRA Locus in Different Cells within a Tumour Sample.

Within T cells and other stromal cells which are known to be diploid there are two copies of the TCRA gene and the copy number around the TCRA locus can be said to equal 2. Assuming that within T cells there is a region within the TCRA gene locus that is always lost during VDJ recombination, this leads to a copy number of 0 in this small genomic segment (as illustrated on FIG. 3B). In tumour cells there can be both copy number gains and losses across chromosome 14 (where the TCRA gene is located) leading to different possible copy number states of the TCRA gene that may differ from 2. The present method assumes that there are no break points within the TCRA gene originating from somatic alterations in cancer cells (although the region as a whole may not be diploid in tumour cells).

As explained in more detail in the two subsections below, the mean copy number at the start of the TCRA gene (chr14: 22090057 (hg19)) is inferred from all cancer cells in the sample using ASCAT, and is referred to as the local tumour copy number at the TCRA gene. This is used as a term in the SCNA-aware TCRA T cell fraction provided by equation (7) below. Additionally, when tumour somatic copy number alteration information is not available, a SCNA-naive TCRA T cell fraction (equation (3)) can be used, and is referred to as the naive TCRA T cell fraction.

Calculation of TCRA T Cell Fraction from WES Data

The ASCAT approach (allele-specific copy number analysis of tumours) described in Van Loo et al. (2010) provides a method for estimating allele-specific copy numbers from DNA extracted from samples that include a mixture of tumour cells and non-aberrant cells. The formula below is the equation used by ASCAT (Van Loo et al., 2010) to estimate tumour ploidy at a genomic location i, in combination with a second equation for the B allele frequency at the location (see Van Loo et al.):

$${r}_{i} = γ {log}_{2} ( \frac{2 ( 1 - p ) + p ( {n}_{Ai} + {n}_{Bi} )}{{Ψ}_{S}} ) \qquad ( 1 )$$

where rᵢ is the log R (a measure of total signal intensity, quantifying the total copy number at a genomic locus) at a location i, γ is a constant dependent on the technology used (for example, a value of γ=1 may be used for Illumina Hiseq), p is the tumour purity (fraction of tumour cells amongst the population of cells in the sample), n_{Ai} and n_{Bi} are the allele specific copy number values, the factor 2 represents the diploid copy number assumed for normal tissue, and Ψₛ is the average ploidy of the tumour sample. When using ASCAT with sequencing data, rᵢ is the log (base 2) of the read depth ratio at genomic location i, where the ratio is between the coverage for a tumour sample and a germline sample at the same genomic location.

In the present work, the aim was to identify the fraction of T cells amongst a population of cells in a sample. Further, this sample may contain aberrant cells (e.g. tumour cells) which may have unknown ploidy at the genomic locus i. By analogy with the mixed population investigated in Van Loo et al., the rᵢ value would represent the ratio of coverage between a sample comprising T cells amongst other (non-T) cells, and a sample without T cells. For example, tumour biopsy samples and matched blood samples are frequently available for tumour patients. In practice, contrary to the tumour situation where a non-tumour sample can be used to quantify rᵢ, no sample from a patient is known to definitely lack any T cells. Indeed, both a tumour sample and a matched blood sample are likely to contain T cells. Thus, the inventors devised a new formula and a way of estimating the log R from a single sample (as explained in detail in the section below). Briefly, this is calculated by using the median coverage of genomic regions at the extreme beginning and end of the TCRA gene as a normal background rate that is assumed to have copy number values unaffected by any VDJ recombination (chr14: 22090057-22298223 and chr14: 23016447-23221076 in hg19). Then, by dividing the coverage across the TCRA genomic region by this median value the inventors create an estimate for r_{T} across the entire TCRA gene (chr14: 22090057-23221076). Thus, the present case can be expressed as:

$${r}_{i} = γ {log}_{2} ( \frac{{Ψ}_{Si} ( 1 - f ) + f ( {n}_{T} )}{{Ψ}_{S}} ) \qquad ( 2 )$$

where f is the T cell fraction of the sample, n_{T} is the copy number of the T cell at the TCRA locus (or any other VDJ locus under investigation, depending on the type of cells to be quantified), γ is a constant dependent on the technology used (for example, a value of γ=1 may be used for Illumina Hiseq), rᵢ is the log R calculated from a single sample as explained below, Ψₛᵢ is the copy number for the sample excluding T cells at the TCRA locus (or any other VDJ locus under investigation), and Ψₛ is the copy number at this locus for the entire sample without taking into account any changes due to VDJ recombination. Assuming that we are looking at the maximum point of VDJ recombination (referred to as r_{VDJ}), it can be assumed that n_{T}≈0. Thus, at this location, equation (2) simplifies to equation (2b):

$$\begin{matrix}{r}_{VDJ} = γ {log}_{2} ( \frac{{Ψ}_{Si} ( 1 - f ) )}{{Ψ}_{S}} ) & ( 2 b ) \\ f = ( {Ψ}_{Si} - {Ψ}_{S} {2}^{\frac{{r}_{VDJ}}{γ}} ) / {Ψ}_{Si} & ( 2 c )\end{matrix}$$

At this point, two different situations may be present: (a) it can be assumed that Ψ_{S}≈Ψ_{Si}—for example in normal samples where the average ploidy will always be 2 (e.g. in the case of a blood sample), or where the average ploidy may not be 2 but the fraction of T cells present is small (e.g. cancer samples with low T cell content); (b) the above assumption does not hold, for example in tumour samples where the T cell content is not small. Case (a) lends itself to a straightforward simplification of equation 2, into equation (3):

$$f = 1 - {2}^{\frac{{r}_{VDJ}}{γ}} \qquad ( 3 )$$

This is referred to as a “naive TCRA T cell fraction” equation. When the assumptions in (a) do not hold (case (b)), the following equations for Ψ_{S} and Ψ_{Si} can be used:

$$\begin{matrix}{Ψ}_{Si} = 2 ( 1 - {p}^{′} ) + {p}^{′} {Ψ}_{T} & ( 4 ) \\ {Ψ}_{S} = 2 ( 1 - p ) + p {Ψ}_{T} & ( 5 ) \\ {p}^{′} = \frac{p}{1 - f} & ( 6 )\end{matrix}$$

where Ψ_{T} is the local tumour copy number at the TCRA locus (calculated as the mean copy number at the start of the TCRA gene (chr14: 22090057 (hg19)) inferred from all cancer cells in the sample using ASCAT), and p′ is the adjusted tumour purity taking into account the absence of any T cell DNA at the location of maximum VDJ recombination. Wr is calculated as the mean copy number from all cancer cells (i.e. across all subclones, where respective subclones may have different copy numbers at the locus) at the start of the TCRA locus (or any other VDJ locus under investigation) because the beginning of the gene is not expected to show any signal from VDJ recombination. Thus, the end of the gene could be used instead of the beginning in any embodiment, without affecting the results, as this also is not expected to show any signal from VDJ recombination. Without wishing to be bound by theory, it is believed that the use of the signal at the beginning of the gene is beneficial because the signal associated with VDJ recombination is closer to the end of the gene than to its beginning (due to the J segments being clustered close to the end of the gene, whereas the V segments are more spread out). Additionally, it would also be possible in any embodiment to use the mean copy number from all cancer cells at a region outside of the VDJ locus under investigation (if such data is available, for example when whole genome sequencing is used rather than whole exome sequencing), such as e.g. before or after the locus. However, increasing distances from the locus of interest increase the likelihood that the signal seen will correspond to a different copy number event in the cancer cells, and hence no longer reflect the tumour copy number at the VDJ locus under investigation.

Substituting equations (4), (5) and (6) into equation (2) produces equation (7):

$$f = ( 1 - p + \frac{p}{2} {Ω}_{T} ) - ( 1 - p + \frac{p}{2} {Ψ}_{T} ) {2}^{\frac{{r}_{VDJ}}{γ}} \qquad ( 7 )$$

Equation (7) represents a “more complete” version of equation (3) (i.e. a solution of equation (2) that does not make all of the assumptions made to arrive at equation (3). Thus, by applying the same assumptions starting from equation (7), it is possible to recover equation (3). In particular, in cases where these are no cancer cells (no cells with a ploidy that differs from the normal 2; p=0), equation (7) immediately simplifies to equation (3). Similarly, where the local copy number around TCRA in any tumour cells present is the same as in normal cells (Ψ_{T}=2) this formula also simplifies to that in equation (3). Thus, Equation (3) can be used to calculate the TCRA T cell fraction in samples from normal tissue or as a naive estimate where the tumour purity or copy number status is unknown.

In other words, the non-cancer component (1−p) of a sample can be rewritten by dividing it into a T cell and non T cell subset:

$$( 1 - p ) = ( 1 - p ) {f}^{′} + ( 1 - p ) ( 1 - {f}^{′} )$$

where f′ represents the fraction of non-tumour cells which are T cells and is related to the total fraction T cell fraction of the sample that are T cells (f) as follows:

$${f}^{′} = \frac{f}{( 1 - p )} .$$

Letting the total copy number of the tumour at genomic loci i equal to Ψ_{T} ignoring allele specific copy number, and the copy number of the T cell compartment equal to n_{T} and the copy number of the non-T cell normal cell compartment to be equal to 2 at all genomic loci leads to the following equation:

$${r}_{i} = γ {log}_{2} ( \frac{{n}_{T} ( 1 - p ) {f}^{′} + 2 ( 1 - p ) ( 1 - {f}^{′} ) + p {Ψ}_{T}}{{Ψ}_{S}} )$$

Now consider the genomic location at the VDJ recombination event at the TCRA gene in T cells, assuming at this location that n_{T}→0, additionally at this location r_{VDJ} is directly estimated from the read depth ratio as described herein. Ψ_{S} is calculated as the average copy number of the sample without taking into account VDJ recombination: Ψ_{S}=2 (1−p)+pΨ_{T}. Therefore, at the genomic loci of VDJ recombination we have the following equation:

$${r}_{VDJ} = γ {log}_{2} ( \frac{2 ( 1 - p ) ( 1 - f ′ ) + p {Ψ}_{T}}{2 ( 1 - p ) + p {Ψ}_{T}} ) .$$

Substituting the equation for f′ into this equation and rearranging the equation for the T cell fraction within a sample can be found to be provided by equation (7) above. Note that if the value of the log ratio r_{VDJ} is >0 then the resulting fraction will be negative. In these cases, it may be assumed that there are no TILs present in the sample and only sample noise is being measured. Thus, f may be set to 0. Conversely if the tumour purity p is known (and/or it can be assumed that there is no error in the calculation of the tumour purity), then any values of f higher than 1−p are not plausible (since the tumour purity+ T cell fraction of a sample cannot be greater than 1) and f can be rounded down to this value if a higher value is obtained.

Calculation of Log Read Depth Ratio from a Single Sample in WES

To calculate the T cell fraction from equation (3) (for a naïve estimate of T cell content) or equation (7) (if tumour copy number and purity are known), the log read depth ratio rᵢ needs to be estimated from the raw coverage data (i.e. from a single sample, rather than from matched samples comprising and not comprising, respectively, a population of cells to be quantified).

In short, this log R is calculated by using the median coverage of genomic regions at the extreme beginning and end of the TCRA gene (or any other VDJ locus under investigation, depending on the type of cells to be quantified) as a normal background rate that is assumed to have copy number values unaffected by any VDJ recombination. As explained above, it would also be possible in any embodiments to use the beginning or end of the VDJ locus under investigation (instead of both), or other (preferably nearby) regions that can be assumed to have copy number values unaffected by VDJ recombination. Without wishing to be bound by theory, it is believed that the use of longer normalisation regions (e.g. using both the beginning and end regions, and/or increasing lengths of the beginning region) advantageously compensates for noise that is typical in read depth data. For example, the beginning region can be extended to include a number of V segments that are unlikely to be frequently lost, or even regions outside of the gene if this data is available (such as e.g. when using whole genome sequencing data). In the present case, the following regions were used: chr14: 22090057-22298223 and chr14: 23016447-23221076 in hg19. Then, dividing the coverage across the TCRA genomic region (or any other VDJ locus under investigation) by this median value results in an estimate for rᵢ across the entire TCRA gene (in this case, chr14: 22090057-23221076 in hg19). The idea behind this is illustrated in FIG. 5.

In more detail, from an aligned BAM file to either hg19 (based on GRCh37) or hg38 (GRCh28) (depending on the data set, e.g. the TCGA data was obtained pre-aligned to hg38, whereas the TRACERx and other data was aligned in house to hg19) the coverage at an individual base level is extracted using the samtools (version 1.3.1) depth function (with parameters-q 20-Q 20). Once this is done, only bases within known exons as defined by the SureSelect human all exons probes (version 5) used within TRACERx, within each exon the reads are then normalised with a rolling median in windows of 50 base pairs (i.e. the median value within each 50 bp window is taken to be the new value). Note that the size of window is not fixed and in embodiments windows of fewer than or more than 50 bp can be used, such as e.g. between 20 and 200 bp, or between 50 and 150 bp. Suitable sizes of windows can be determined empirically for a particular data set, type of data or context, by comparing the lymphocyte fraction estimates obtained using the present method with various candidate window sizes to orthogonal metrics of lymphocyte fractions such as the Danaher score.

The median baseline coverage value is calculated in regions of TCRA assumed not to be affected by VDJ recombination, taking regions at the beginning and end of the gene (chr14: 22090057-22298223 and chr14: 23016447-23221076 in hg19-corresponding to the first 12 V segments and the C segment of TCRA). All coverage values are then divided by this value, with the log then being taken to calculate a ‘single sample’ log ratio. To complete the calculation of r_{VDJ} a general additive model is fitted to the data with the average value of the model at the location of maximum VDJ recombination being taken as the value for r_{VDJ}. A generalized additive model is a generalized linear model in which the linear predictor is given by a sum of smooth functions of the covariates plus a conventional parametric component of the linear predictor. The model effectively fits a smoothed line to the data, and any approach to do this may be used in the context of the disclosure (e.g. using any types of generalised linear model or any other smoothing approach such as e.g. fitting a piecewise constant model). In this case, the model was fitted using the “geom_smooth” function in R, which uses the “gam” function from the mgcv package (mgcv::gam) with formula y˜s(x, bs=“cs”) (see https://rdrr.io/cran/mgcv/man/gam.html for details). This implementation represents the smooth functions of the generalised additive model using penalised regression splines. A diagram of this process is shown on FIG. 5. The location of maximum VDJ recombination was defined as the gap between the final TCRA V segment and the first segment of the TCR delta gene, rounded to have a size of 80000 bp (80k bp) and start at position 22800000 (leading to the region chr14: 22800000-22880000)—as explained below. Other definitions are possible and small changes in the size of this region are not expected to make much difference. Regions of maximum VDJ recombination may be defined for other VDJ loci using a similar approach, i.e. by defining a region located at least partially around or within the region between the final V gene segment and the first J gene segment, such as a region corresponding to the gap between the final V gene segment and the first J gene segment, optionally rounded for ease of manipulation, such as e.g. to the nearest k bp or the nearest 10k bp.

In summary, for many tumour samples, equation (7) can be used, requiring knowledge of both tumour purity and the tumour copy number at the TCRA gene. In the special case when the copy number at TCRA is exactly 2, as is true in all blood derived samples and many tumours, equation (3) can be used to calculate the T cell fraction, and is exact. Additionally, if the tumour purity and tumour copy number at TCRA are unknown, equation (3) can function as a naïve estimate for T cell fraction (although depending on whether the above mentioned assumptions hold true or not, this naïve estimate may not be exact). This is investigated in the next section.

### Naïve TCRA T Cell Fraction Vs Exact Estimate of T Cell Fraction

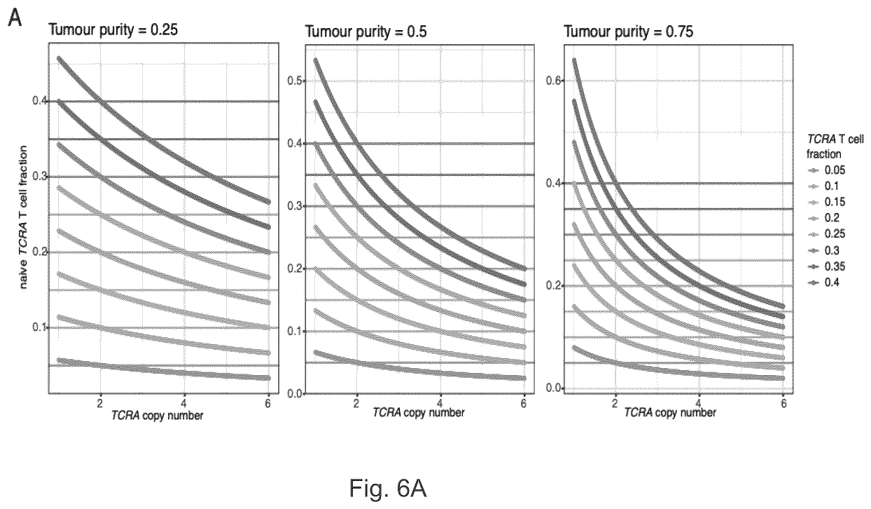

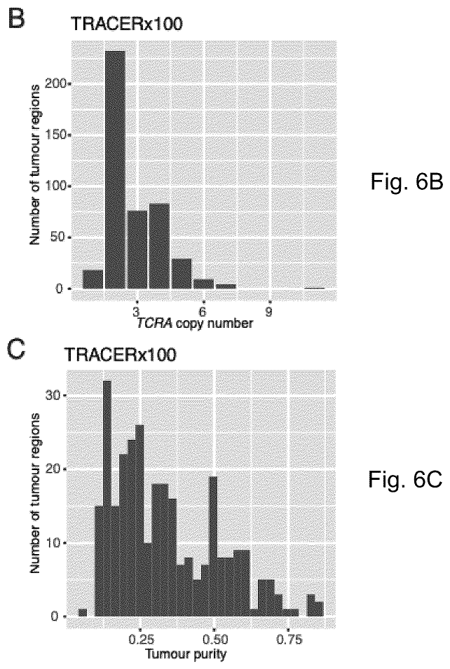

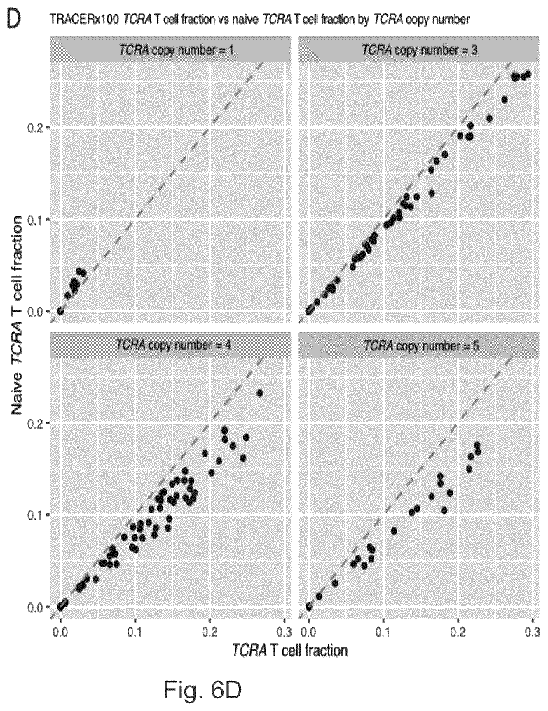

FIG. 6A shows the (theoretical) difference between the estimated naïve value for T cell against different real T cell fraction values for a range of local copy number values and tumour purities based on theoretical values as derived from equations (2), (3) and (7). At low T cell fractions and local copy number values close to 2 this estimate is very accurate. FIGS. 6B-C shows using real data from the TRACERx100 cohort (more information on this data is provided in Example 2) that the distribution of local TCRA copy number has a mode of 2, while tumour purity values are typically low. For cases where the local TCRA copy number is not 2, FIG. 6D shows the correlation between the naïve estimate and exact calculated T cell fraction.

Optimisation of Segments Used for Estimation of the Log Ratio (r_{VDJ})

The calculation of r_{VDJ} requires a choice of (i) a segment of maximum VDJ recombination, and (ii) a segment to be used to calculate the “normal baseline” to which the coverage across the TCRA genomic region is compared (by calculating a ratio, as explained above).

For the focal segment representing the position of maximum VDJ recombination, a gap between the final V gene segment and first segment that encodes part of the TCRδ chain was used (hg19, chr14: 22800000-22880000). This region of the genome theoretically is likely to be within the T cell receptor excision circles (TRECs) and overlaps with the region previously used for PCR primers measuring TRECs (Kuss et al., 2005). As explained above, any region between the final V segment and the first J segment of the VDJ locus of interest may be used.

Due to sequencing noise it is desirable to have as wide a region as possible in the calculation of the normal baseline using V gene segments unlikely to be commonly lost in VDJ recombination (see Calculation of log ratio from a single sample in WES). However, the more V gene segments chosen the more likely that T cell clonotypes exist where these gene segments have been excised by TRECs (such that the coverage in the region would then be influenced by VDJ recombination).

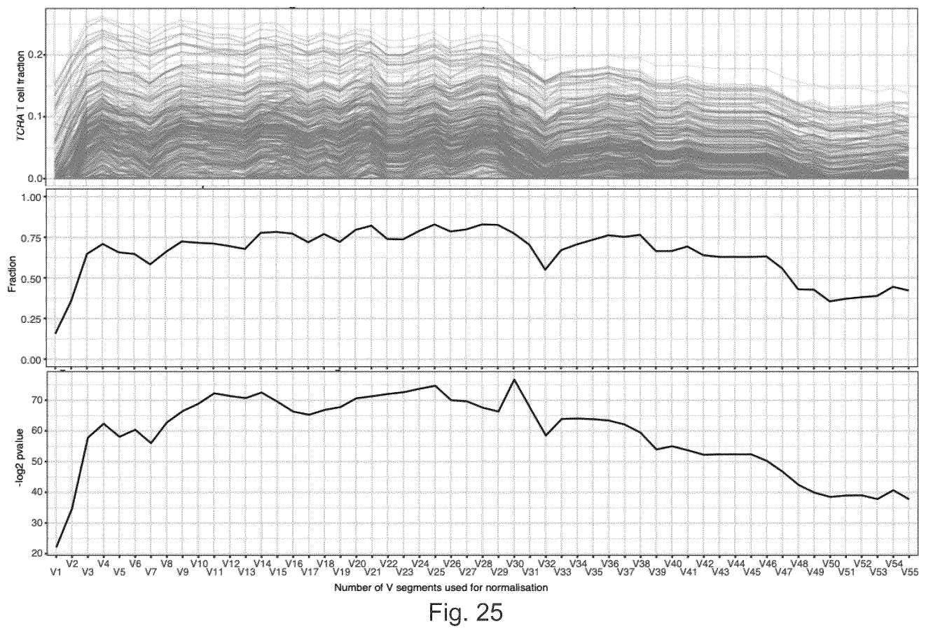

For the local segments an optimisation schema outlined below was used to select the first n-V gene segments as well as the final C segment. Taking the first n-V segments in turn, a score (TCRA T cell fraction, f, non-GC corrected) was calculated for the whole TRACERx100 cohort (see more detail on this cohort in Example 2). The number of V segments was chosen based on which n had the most non-zero values of f and maximised the association between the calculated T cell fraction and the Danaher transcriptomic score for T cell infiltrate (a comparative metric of T cell infiltration, see Example 2). The length of segments used were found to reduce the levels of noise (i.e. the more segments used the better for normalisation from a noise point of view) but, if too long in some cases, would overlap with segments deleted from certain T cell clonotypes (i.e. longer segments are more likely to include one or more segments lost in one or more T-cell clonotype(s)). Therefore, in order to be conservative the first 12 V segments were chosen to be used as a compromise between high association with the Danaher scores and being as short as possible (as illustrated on FIG. 25, where the top plot shows the TCRA T cell fraction as a function of the number of V segments used for normalisation, in the TRACERx100 data, the middle plot shows the corresponding fraction of non-zero samples, and the bottom plot shows the significance of the correlation with the Danaher T cell signature, assessed using the Spearman's method). Note that other numbers of segments such as e.g. the first 11 V segments could have been used without significant impact on the results.

GC Normalisation Method Used within TIL ExTRECT

GC content is known to bias sequence coverage values. Thus, TIL ExTRECT can normalise the coverage values by GC content before calculation of the T cell fraction. To do this GC content is calculated at two scales, the local exon level where GC content for each TCRA exon (GCₑₓₒₙ) is calculated and a larger macro scale (GC_{macro}). For this larger scale the GC content is calculated in 1000 bp windows across the entire TCRA gene. A general additive model is then fitted to this data to create a smoothed value for the macro scale GC content across the TCRA gene. To normalise for GC content the coverage values of every base-pair within all TCRA exons are put into the following linear model:

$$lm ( basepair ∼ {GC}_{exon} + {GC}_{exon}^{2} + {GC}_{macro} + {GC}_{macro}^{2} )$$

The residuals of this model are then taken as the new GC normalised coverage values. Unless stated otherwise all TCRA T cell fractions used within this paper have been GC corrected.

Following GC correction the baseline of scores are re-adjusted by taking the median value at the beginning and end of the TCRA gene (chr14: 22090057-22298223 and chr14: 23016447-23221076 in hg19) as well as the median value of the fitted generalised additive model (GAM) at the location of maximum VDJ recombination (chr14: 22800000-22880000). As it is theoretically impossible for the T cell fraction to be negative and by necessity for this to be so the log read depth ratio at chr14: 22800000-22880000 must be ≤0, the maximum of the two median values is taken as the new baseline and set to 0.

Calculation of Confidence Intervals within TIL ExTRECT

95% confidence intervals for the TCRA T cell fraction was calculated taking two factors into account, 1) the noise in the final baseline adjustment post GC corrections, 2) the uncertainty in fitting the GAM model used in the final calculation of the TCRA T cell fraction.

To account for the noise in the baseline adjustment, the 95% confidence interval for the adjustment value was calculated as 1.96 times the standard deviation of the coverage values in the region used for normalisation. For the fitting of the GAM model, the R package ‘gratia’ (v0.5.1) was used to calculate the simultaneous confidence intervals (using the confint function). Finally these two sources of uncertainty were combined to generate the 95% confidence intervals.

### TCRA T Cell Fraction Bias Due to Over-Estimated Tumour TCRA Copy Number

Over estimated values for TCRA tumour copy number will lead to an inflated value for the TCRA T cell fraction. This can occur due to a poor quality solution choice from copy number segmentation algorithm such as ASCAT. One indication that this is the case is if the calculated TCRA T cell fraction from TIL ExTRECT exceeds the value of 1-tumour purity. In these cases the given copy number solution is deemed unreliable and instead the naive estimate for T cell fraction that assumes that the TCRA tumour copy number is 2 is used as an alternative solution.

### Example 2—Validation of WES Derived TCRA T Cell Fraction

### Introduction

To evaluate the accuracy of the TCRA T cell fraction metric described in Example 1, the inventors used four orthogonal approaches.

First, as a positive and negative control of T cell content presence, WES data from cell lines was used. Second, simulated next generation sequencing (NGS) data with a range of T cell fraction values were used. The simulated data was also used to investigate the effect of local tumour TCRA copy number and tumour purity on the accuracy of the score. Third, the inventors examined how the score compares to orthogonal immune related data within multiple NSCLC cohorts. Fourth, the inventors calculated how similar their score was to an alternative DNA based method for inferring immune content.

### Results

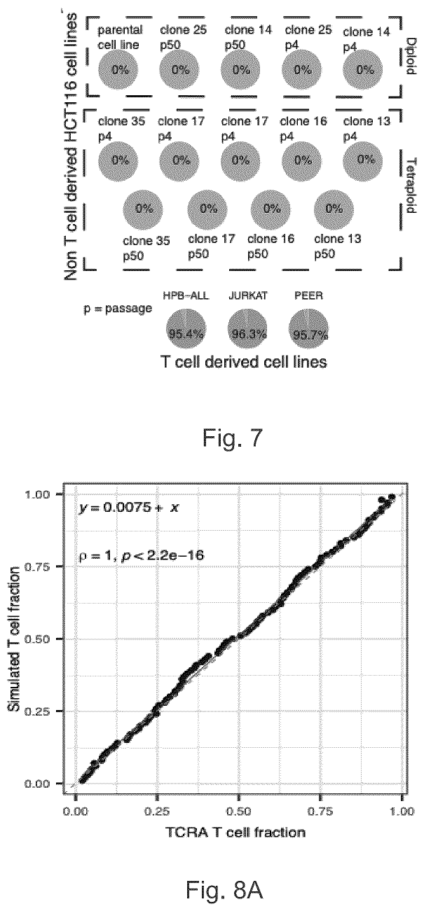

As a first validation of the approach described in Example 1, the inventors utilised WES data a from cell lines comprised of 14 independent samples derived from the HCT116 cell line, including tetraploid and diploid clones, with varying degrees of genomic complexity (López et al., 2020) and three samples derived from cell lines originating from T cell lymphomas (JURKAT, PEER and HPB-ALL) from the cancer cell line encyclopaedia (Ghandi et al., 2019). In theory, the TCRA T cell fraction should be zero in the NGS from the HCT116 cell line while the T cell lymphoma derived cell lines, which should have all undergone VDJ recombination, should have a TCRA T cell fraction of 1 (reflecting 100% of cells as T cells). Reassuringly, the HCT116 cell lines all had a calculated fraction of 0, regardless of their diploid or tetraploid status (as illustrated on FIG. 7). Conversely, the three T cell derived cell lines had scores calculated with the naïve estimate to be close to 1 (˜0.95-˜0.96) (see FIG. 7). This slight difference from an estimated fraction of exactly 100% is likely to be explained by technical (e.g. misaligned reads) or biological factors.

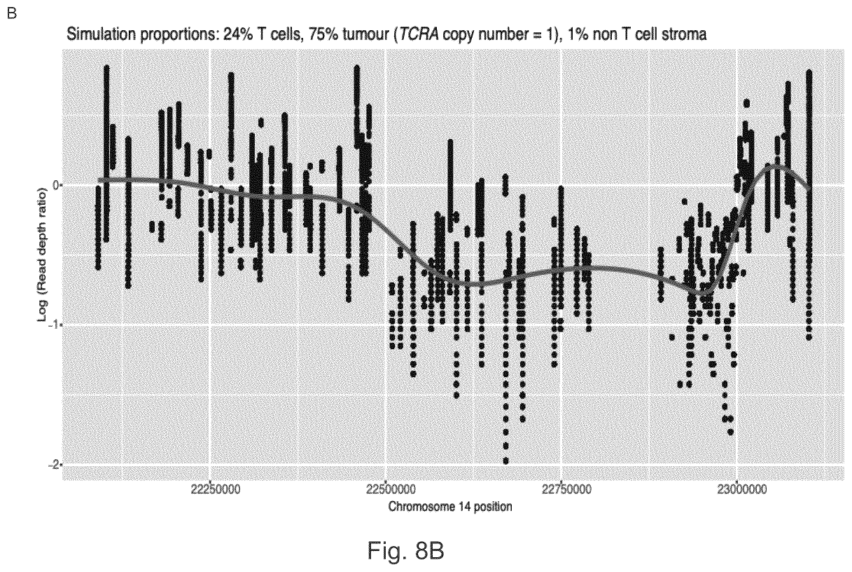

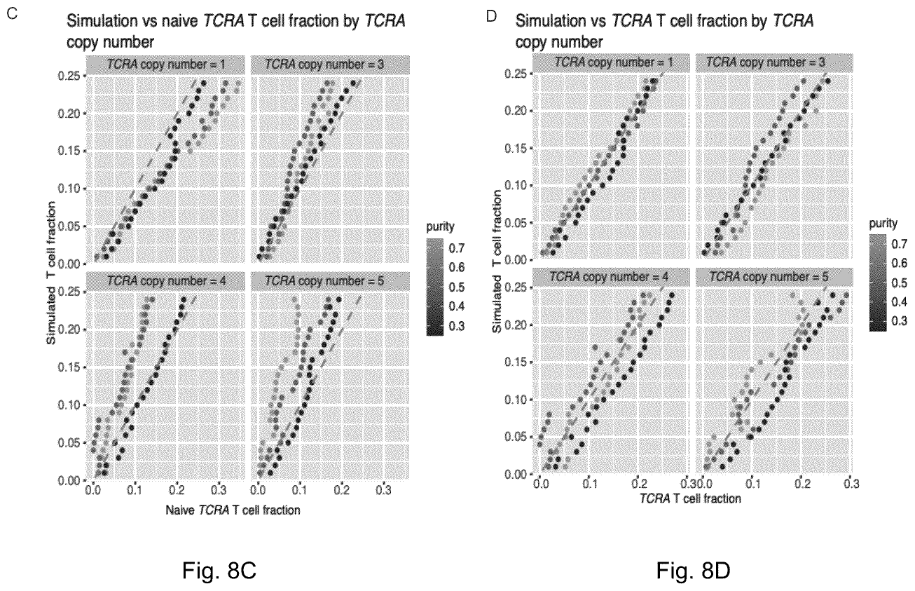

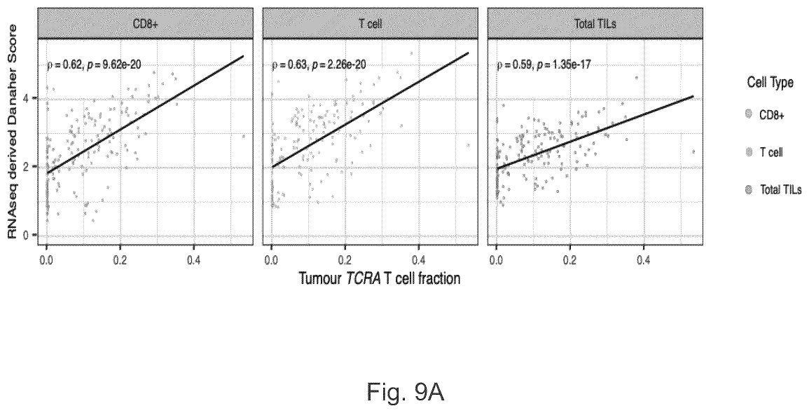

As a second validation approach, to further evaluate the accuracy of the TCRA T cell fraction beyond binary presence or absence, and for a range of T cell fractions, simulated NGS data was obtained (see Methods) with a range of T cell fraction values. As can be seen on FIG. 8A, there was a highly significant, near perfect, relationship between simulated and calculated T cell fraction in samples with a background TCRA copy number of 2 (ρ=1, ρ<2.2e−16). Further investigation of the effect of local tumour TCRA copy number (see Methods) and tumour purity on the accuracy of the score was investigated with simulated data, and also demonstrated a significant relationship between the simulated and inferred TCRA T cell fraction (as shown on FIG. 8C-D).

As a third validation approach, the inventors examined how the score compares to orthogonal immune related data within the TRACERx100 cohort and also within the lung adenocarcinoma (LUAD) TCGA (Cancer Genome Atlas Research Network, 2014) and lung squamous cell carcinoma (LUSC) (Cancer Genome Atlas Research Network, 2012) cohorts. These NSCLC cohorts have had estimates for T cell content from a range of different data types, including slides and RNA-seq, histopathological methylation data.

The TRACERx100 cohort is made up of 327 tumour regions subjected to WES derived from 100 tumours from 100 patients each with a matched germline blood sample also subjected to WES (see Table 1). 189 of these tumour regions also had matched RNA-seq data from which transcriptomic-based immune scores could be calculated.

TABLE 1
Overview of samples in the TRACER × 100 cohort.

| TRACER × 100 | LUAD | LUSC | Other | Total |
|---|---|---|---|---|
| Number of patients | 61 | 32 | 7 | 100 |
| WES regions | 181 | 116 | 30 | 327 |
| RNA-seq regions | 105 | 51 | 20 | 176 |
| TIL regions | 127 | 96 | 23 | 246 |
| TIL and RNA-seq regions | 81 | 49 | 18 | 148 |

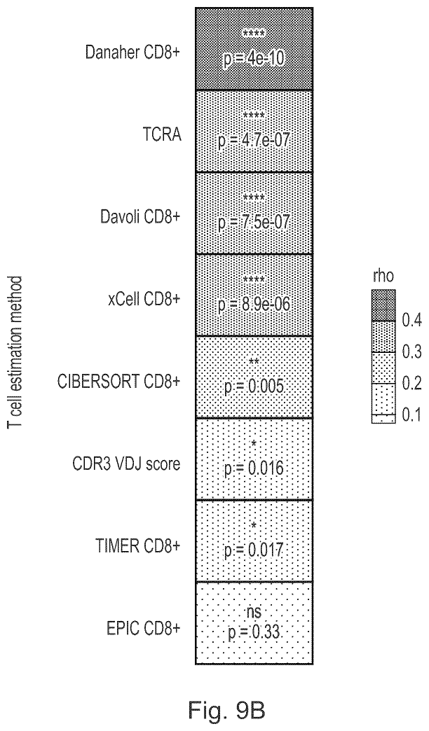

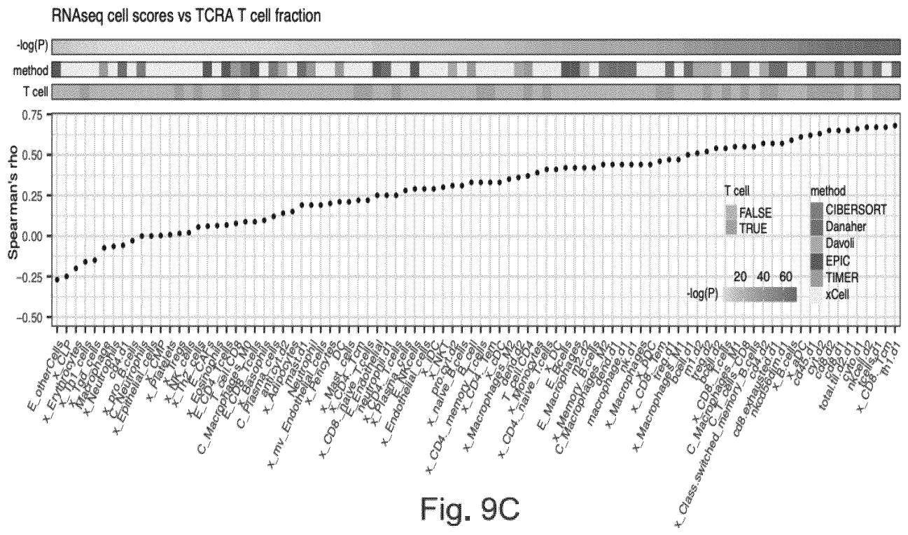

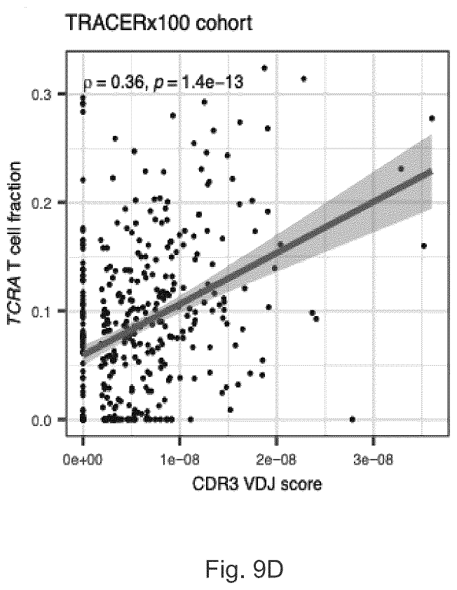

In particular, the inventors examined how the score compares to orthogonal non-WES immune-related data within the TRACERx100 cohort. Immune-related signature scores for various cell types from Danaher et al., Davoli et al., xCell, TIMER, EPIC and CIBERSORT were calculated for the 189 TRACERx100 regions with RNA-seq data (Rosenthal et al., 2019). The TCRA T cell fraction from WES was found to have a significant positive relationship with multiple immune scores derived from the different methods. The top three most strongly related terms were all T cell related (Danaher Th1: ρ=0.68, P=9.0e−23, xCell CD8 T cell memory ρ=0.67, P=2.3e−22 and Danaher T cell: ρ=0.67, P =2.94e−22). Other non-T cell scores also ranked very highly such as the Davoli score for NK cells (ρ=0.67, P=3.92e−22) and the xCell identified activated dendritic cells (ρ=0.61, P=1.26e−17), indicating the complexity of the tumour immune microenvironment and that T cell infiltration is strongly correlated with infiltration from other immune cells. The TCRA T cell fraction from WES was found to have a significant positive relationship with the Danaher transcriptomic signatures corresponding to CD8+ cells (ρ=0.62, P=9.6e−20), T cells (ρ=0.63, P=2.3e−20), and total TIL (ρ=0.59, P =1.4e−17) (as shown on FIGS. 9A, 9C).

To further confirm the TCRA T cell fraction, the inventors evaluated its association with TIL scores based on histopathological H&E slides that were manually examined and scored by a pathologist available for 88 patients. Reassuringly, the TCRA T cell fraction significantly correlated with pathology TIL fraction estimates. Notably, the pathology TIL fraction estimates will not exclusively include CD8+ and CD4+ T cells, and thus a perfect correlation between it and the TCRA T cell fraction is not expected.

As a fourth approach, the inventors calculated how similar their score was to an alternative DNA based method of inferring immune content (Levy et al), based on the number of reads in each WES that align to the CDR3 region following VDJ recombination relative to total coverage (CDR3 VDJ score, see Methods for details). In the TRACERx100 cohort the inventors observed a significant positive correlation between TCRA T cell fraction and the CRD3 VDJ score (FIG. 9D, ρ=0.36, P=1.4e−13). However, we noted that despite the high coverage in the TRACEERx100 cohort, the CDR3 VDJ score is significantly constrained by sequencing depth; the number of reads aligning to the CDR3 region is typically very low (Q1=0, medium=2, mean=2.335, Q3=3, maximum =14). To explicitly quantify the extent to which T cell ExTRECT derived TCRA T cell fraction as well as the CDR3 VDJ score is robust to differences in sequencing coverage the inventors employed two complementary methods (described below).

Next, selecting the subset of tumour regions with both RNA-seq data and pathology-derived TIL scores (147 regions), the inventors were also able to evaluate how both the TCRA T cell fraction, CDR3 VDJ score and six RNA-seq based immune measures for CD8+ cells (Danaher, Davoli, xCell, TIMER, CIBERSORT and EPIC) compared to the TIL scores (as illustrated on FIG. 9B). The Danaher CD8+ score had the strongest association (ρ=0.49) with pathology TILs, followed by our TCRA T cell fraction (ρ=0.41), Davoli (ρ=0.4), xCell (ρ=0.36), CIBERSORT (ρ=0.23), TIMER (ρ=0.2), CDR3 VDJ score (ρ=0.2) and EPIC (ρ=0.082), with all but EPIC being significantly associated. Thus, the TCRA T cell fraction estimation is a vast improvement on alternative DNA based measures and competitive with state-of-the-art RNA-seq based immune signatures in revealing immune cell tumour infiltration content but, unlike many existing methods, provides a precise point estimate of T cell fraction.

Additionally, the inventors further evaluated TIL ExTRECT in an independent NSCLC dataset with lower coverage (median coverage: LUAD =84, LUSC=88.2, compared to a median depth of 426× in TRACERx) from the TCGA. The samples making up the LUAD and LUSC cohort are described in Table 2. The inventors correlated the TCRA T cell fraction with the immune related data from Thorsson et al (Thorsson et al., 2018). Thorsson et al. calculated a measure of total TILs in a sample called “leukocyte fraction” from DNA methylation, as well as an RNA-seq CIBERSORT based T cell CD8 fraction.

TABLE 2
Overview of samples in the TCGA LUAD
and LUSC cohort.

| TCGA | LUAD | LUSC |
|---|---|---|
| Number of patients | 582 | 502 |
| Primary tumour WES | 633 | 558 |
| Normal blood WES | 441 | 316 |
| Normal tissue WES | 224 | 227 |
| RNA-seq tumour | 457 | 486 |
| Methylation tumour data | 573 | 489 |
| RNA-seq and methylation | 455 | 485 |

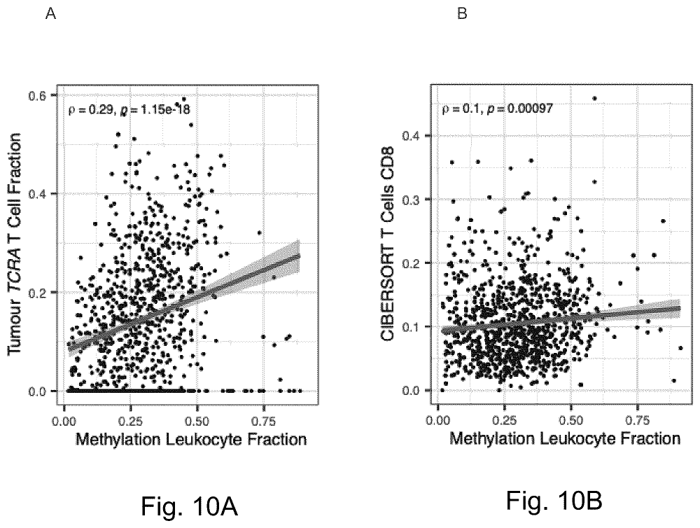

Compared to the CIBERSORT based CD8+ T cell estimate, the TCRA T cell fraction had a stronger association with methylation-derived leukocyte fraction used by Thorrson et al (as shown on FIG. 10, ρ=0.29, P=1.2e−18 vs. ρ=0.1, P=0.00097), indicating both that TCRA T cell fraction correlates well with a methylation-based measure of total TILs and that it is potentially superior to certain RNA measures of T cell content.

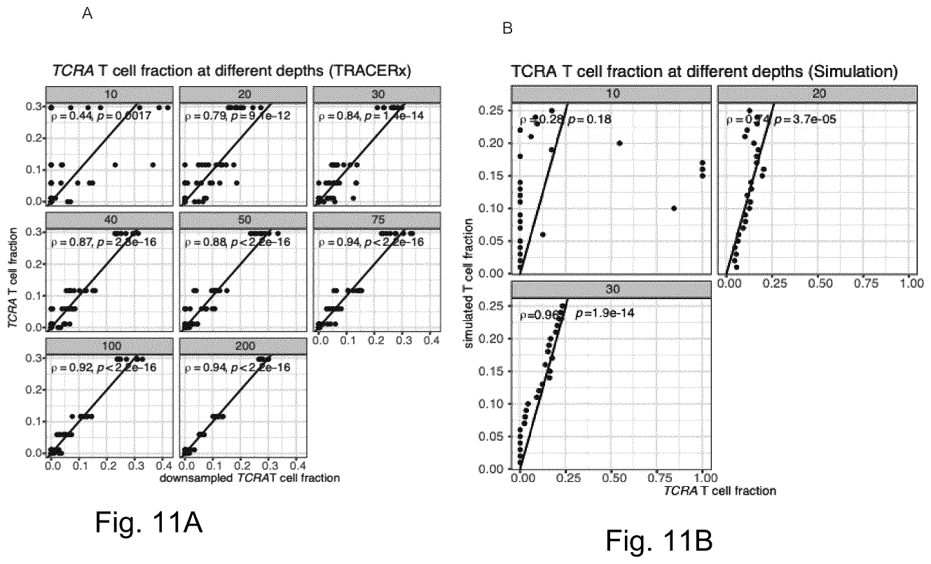

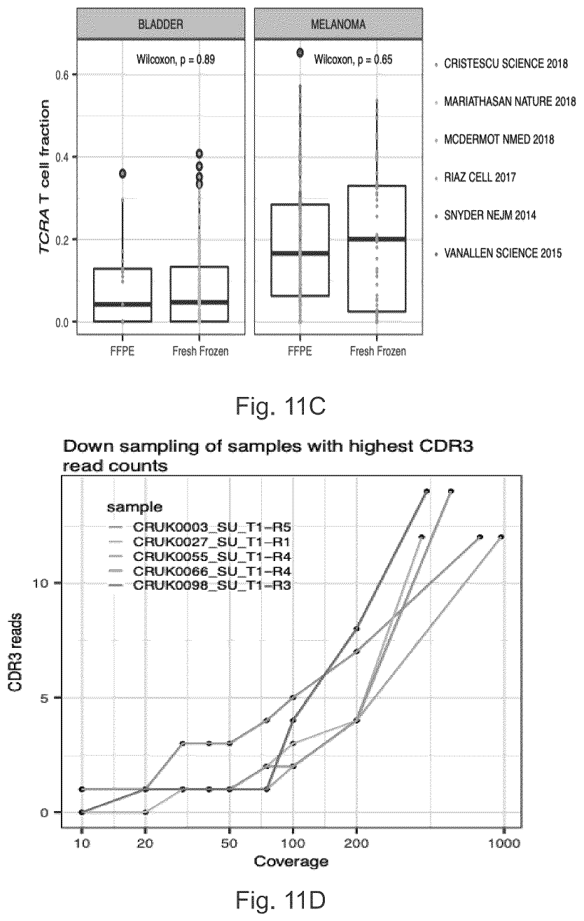

Additionally, the inventors sought to test the extent to which TIL ExTRECT derived TCRA T cell fraction is robust to differences in sequencing coverage. Specifically, to ascertain the minimum sequencing depth required to yield an accurate T cell fraction estimates they employed two complementary methods. First, five NSCLC tumour regions from the TRACERx100 were chosen with TCRA T cell fraction from TIL ExTRECT ranging from 0 to 0.35. These regions were then down sampled to 5, 10, 20, 30, 40, 50, 75, 100 and 200× coverage independently 10 times. The inventors found that the T cell fraction estimates remained consistent at coverage above and including 30× (ρ=0.84, P=1.4e−14) (as shown on FIG. 11A). Consistent with this, the simulated data showed reliable correspondence between 30× coverage and the known T cell fraction (as shown on FIG. 11B, ρ=0.96, P=1.9 e−14). At 20× coverage and below, the inventors observed that the TCRA T cell fraction begins to lose fidelity, although useful information may be obtainable even with coverage of 10× (as shown on FIG. 11A); this is likely due to increasing noise, particularly for lower T cell fractions which start to fall below the limit of detection. Thus, coverages below 30× (including 10× and 20×) may still be useful to distinguish between high and low T cell fractions, but may not be sufficient to obtain reliable estimates of very low T cell fractions. As T cell fractions below 0.1 are common, the use of coverages of 30× and above may be advantageous in providing additional certainty and broad applicability. By contrast, the inventors found that the CDR3 method for determining T cell infiltrate was heavily skewed by low sequencing coverage, selecting the five samples with the highest CDR3 reads and down-sampling to 50× coverage there was one sample with 3 reads detected and the remaining with only a single CDR3 read (FIG. 11D). Consistent with this, it is notable in the TCGA BRCA analysis by Levy et al. that in 56% of the tumours no CDR3 reads were identified.

Finally, from testing on multiple different datasets the inventors identified no systematic significant difference in T cell fraction dependent on whether samples were fresh frozen or formalin-fixed paraffin-embedded (FFPE) (See methods and FIG. 11C) implying that the method is not sensitive to any FFPE-specific DNA alterations.

Thus, the data demonstrates that the TCRA T cell fraction estimation method described herein is an improvement on alternative DNA based measures and competitive with state-of-the-art RNA-seq-based immune signatures in revealing immune cell tumour infiltration content but, unlike many existing methods, provides a precise point estimate of T cell fraction. Further, T cell ExTRECT can be applied to any sample subject to WES, thus permitting an analysis of T cell fraction in both tumour and blood samples.

### Methods

### Statistics

All statistical tests in this example and all others were performed in R 3.6.1. No statistical methods were used to predetermine sample size. Tests involving correlations were done using ‘stat cor’ from R package ggpubr (v0.4.0) with the Spearman's method. Tests involving comparisons of distributions were done using ‘stat_compare_means’ using either ‘wilcox.test’ using the unpaired option, unless otherwise stated. Effect sizes for the corresponding Wilcoxon tests were measured using the ‘wilcox_effsize’ function from the rstatix package (v0.6.0). Hazard ratios and p values were calculated with the ‘survival’ package (v3.2-3) for both the Kaplan Meier curves and Cox proportional hazard model. For all statistical tests, the number of data points included are plotted or annotated in the corresponding figure.

### Definition of Immune Hot and Cold Tumour Regions

Throughout these examples, an immune hot region was defined as one that contains greater than 10% T cells as measured by TIL ExTRECT and an immune cold region was defined as one that contains less than 10% T cells. This threshold was defined based on expert knowledge, and others could be used depending on the context and the purpose of the analysis.

### Fresh Frozen Vs FFPE Samples

To test that the TCRA T cell fraction was reliable and consistent for both fresh frozen and FFPE samples the non-GC corrected TCRA T cell fractions were calculated for six different studies within the CPI response cohort. Three of these studies utilised WES derived from FFPE tissues (n=460) while the other three utilised WES samples derived from fresh frozen tissue (n=357). Fitting a linear model to predict TCRA T cell fraction by histology and FFPE status (Table 6) revealed that cancer type was the main driver of this significance with FFPE status not being significant. Additionally, for melanoma and bladder tumours that had FFPE and fresh frozen WES samples there was no significant difference found (FIG. 11C). It can thus be concluded that whether a WES sample is derived from fresh frozen or FFPE tissue does not significantly affect the values of the TCRA T cell fraction calculated by TIL ExTRECT.

TABLE 6
Summary of linear model for prediction of non-GC corrected
TCRA T cell fraction from histology and FFPE
sample status within the CPI cohort.

| | TCRA T cell fraction | | |
|---|---|---|---|
| Predictors | Estimates | CI | p |
| (Intercept) | 0.07 | 0.04-0.10 | <0.001 |
| Histology [Breast] | −0.01 | −0.09-0.06 | 0.760 |
| Histology | −0.05 | −0.16-0.07 | 0.397 |
| [Colorectal] | | | |
| Histology | 0.05 | 0.01-0.10 | 0.010 |
| [Head and neck] | | | |
| Histology [Lung] | 0.00 | −0.13-0.13 | 0.977 |
| Histology [Melanoma] | 0.12 | 0.09-0.15 | <0.001 |
| Histology [Other] | 0.00 | −0.04-0.04 | 0.982 |
| Histology [Renal] | 0.04 | 0.01-0.07 | 0.009 |
| FFPE status | 0.01 | −0.02-0.04 | 0.589 |
| [FreshFrozen] | | | |

| | |
|---|---|
| Observations | 817 |
| R²/R² adjusted | 0.144/0.136 |

### Simulated Data

Simulated data to test the results of the non-GC-corrected TCRA T cell score was created using ART Illumina (version 2.5.8) (Huang, Li, Myers, & Marth, 2012) combined with tools from the MASCOTE (Zaccaria & Raphael, 2020) tumour simulation method. In short, ART was used to generate simulated paired end FASTQ files for human chromosome 14 as coming from a HiSeq 2500 DNA sequencer with coverage of 30 and read length 150 (parameters-p-ss HS25-f 30-na-1 150-m 200-s 10, where-ss refers to the sequencing platform, -f to the coverage, -l to the read length, -m to the mean size of DNA fragment, -s to the standard deviation of fragment size and -na indicates that no output alignment file is to be provided see https://manpages.debian.org/stretch/art-nextgen-simulation-tools/art_illumina. 1 for details). These FASTQ files were then aligned to the human genome hg19 with bwa mem (v 0.7.15). Picard tools (version 1.107) were then used to clean, sort and index the resulting BAM file. Since only coverage at the TCRA gene was relevant for the testing of the simulated data a BAM file was created with samtools (version 1.3.1) view of all reads mapping to chr14: 20000000-24000000. This BAM file served as the template for the normal and tumour cells. A T cell template was created from this BAM file by only extracting reads that mapped to chr14: 20000000-22500000 or chr14: 23000000-24000000 and merging the results into a single BAM file with samtools merge. This T cell BAM file therefore has a manually created 100% deletion between the positions chr14: 22500000-23000000. Any read that mapped partially to either of the regions on either side of this gap was included (i.e. not deleted). Since read sizes are 150 bp, the presence or absence of these reads is believed to be insignificant compared to the size of the deletion. Using this T cell and normal/cancer BAM files different mixed BAM files were generated using the MixBam.py module used within the MASCOTE method. This creates mixed BAMs by sampling of the normal/cancer and T cell BAMs depending on both the genome length and copy number of the mixture. In this way it was possible to create simulated data containing differing proportions of normal, T cell, and cancer cell populations with different cancer somatic copy number alteration values.

Simulated data using this method was created for all the possible combinations of a tumour purity=[0.25, 0.5, 0.75], a tumour copy number=[1, 3, 4, 5] and a T cell fraction=[0.01 to 0.25 by 0.01], as well as for tumour copy number 2 with T cell fractions ranging from 0.01 to 0.99 by intervals of 0.01. Following creation of this simulated data, the T cell fraction was calculated only using the reads from positions within exons of the TCRA gene as is done in the standard TIL ExTRECT method applied to WES (as described in Example 1). An example of the output for a single simulated run with 24% T cells and 75% tumour with a local copy number around the TCRA of 1 is given in FIG. 8B, while FIGS. 8C and D show the difference between simulated and calculated values with different local tumour copy number and purity values. The naïve score overestimates the T cell fraction when the copy number is below 2 and underestimates it when it is above 2. However, the exact score follows the line y=x, indicating that it is indeed exact.

### Investigating the Effect of Sequencing Depth on TCRA T Cell Fraction Estimation

To assess the effect of lower coverage, five TRACERx100 regions were chosen on the basis of the range of TCRA T cell fractions they represented (high=0.35, medium=0.156, low=0.053, very low=0.010, none=0). For simplicity all regions had a local TCRA tumour copy number of 2. TRACERx100 as a study has median coverage of 430.91×. Although these samples had a range of depths, samtools view was used with the -s option to down sample the aligned BAM files to a specific depth, taking account of the original depth of the region. In this way 10 down-sampled depths created from different random seeds were created to make BAM files at depths of 200, 100, 75, 50, 40, 30, 20, 10 and 5× coverage. Following the down sampling to produce these BAMs, the non-GC-corrected TCRA T cell fraction was estimated using the method described above.

### Capture Kit Exon Bias Detection and Quality Control of Exons Used in Calculation of TCRA T Cell Fraction

As a general quality control step every exon was checked for sufficient coverage before being used in the calculation of the TCRA T cell fraction. Exons with a median value of less than 15× were removed, and if more than 30 exons were below this threshold the sample was marked as failed due to low coverage.

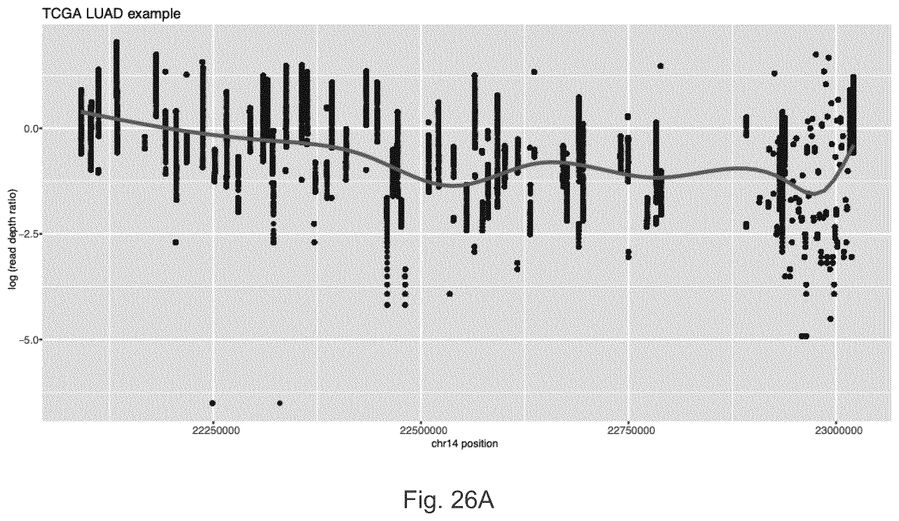

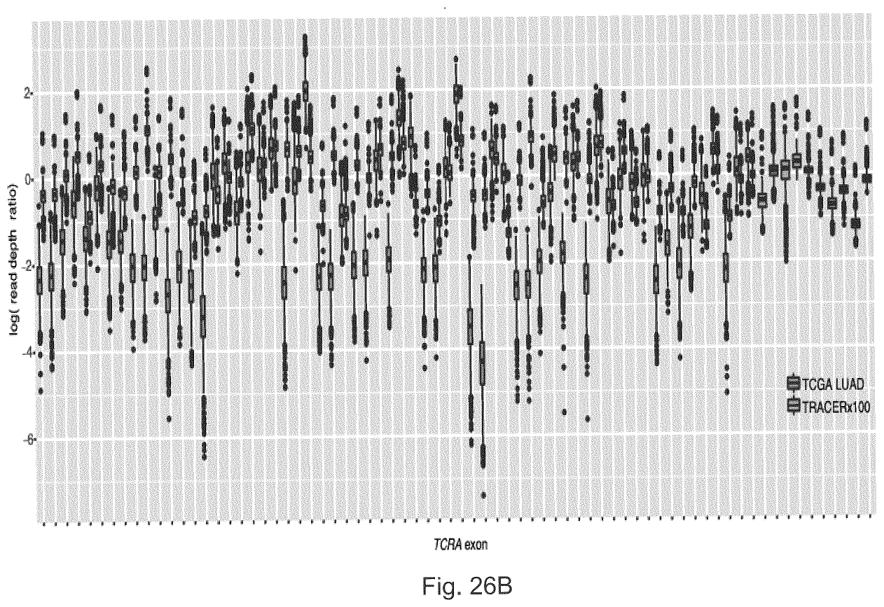

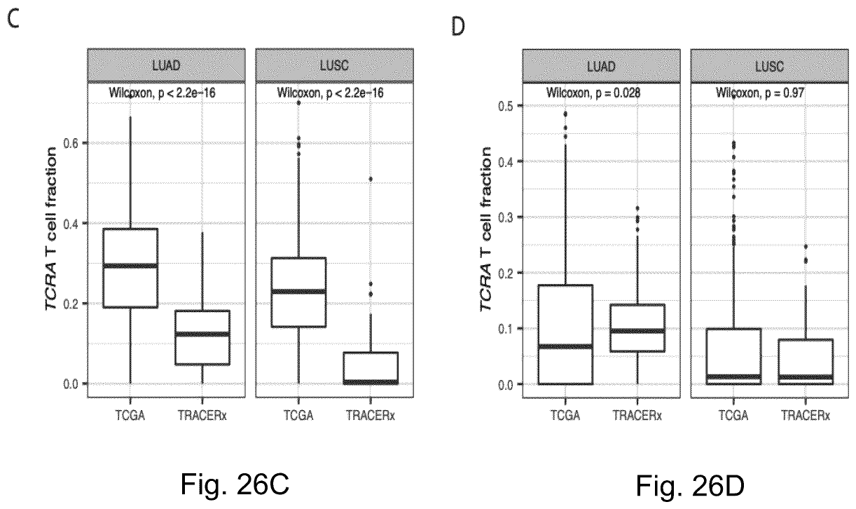

In the course of analysing the TCGA dataset a bias due to the capture kit used (Broad custom set based on Agilent SureSelect v2) was observed. This bias has previously been described by Wang et al (Wang, Kim, & Chuang, 2018) affecting 4833 genes including the TCRA. This bias led to coverage in certain exons being much lower than expected and thus interfering with the calculation of the score. An example of this bias can be seen in FIG. 26A. To resolve this issue, the inventors calculated the average log R ratio in each individual exon (i.e. the average of the log ratio of the read coverage in the exon vs the median read coverage in the first 12V segments and the C segment—as explained above) within the TRACERx100 and TCGA LUAD cohorts (see FIG. 26B). The TCGA LUSC cohort was not used for this analysis although the inventors do not expect the results from this cohort to differ significantly from those for the TCGA LUAD cohort. Exons which had a difference in their medians of >0.5 (where the median is calculated across all samples in each cohort, for each exon, then the difference in medians between the cohorts is examined) were flagged and removed from the TCGA analysis. This led to the exclusion of 99/192 exons. Note that these exons were removed in the TRACERx100 data in the analysis below (even though this data is not expected to suffer from the same bias as the TCGA data, as the bias is believed to be associated with the capture kit used) to show that there is minimal difference in using this reduced set when there is no bias (compared to the big effect that can be observed when the bias exists). Other cohorts, particularly within the CPI dataset (Rizvi et al., 2015; Shim et al., 2020) that used Agilent SureSelect v2 capture kits were also observed to have the same bias (i.e. a bias affecting the same exons—as shown by calculating the median values for all exons as described above and examining the correlation between these and the median values of exons from the data set affected by bias, i.e. the TCGA LUAD cohort in this case) and had scores calculated with this reduced exon set. Additionally, post-GC correction there was a bias observed in a single exon when using the Agilent SureSelect v2 capture kits within one of the TCRδ gene segments. Since this segment is within the focal region used to calculate the TCRA fraction and due to the smaller number of exons in the reduced set, any small change in this exon can create a large effect on the calculated score. Post-GC correction this was found to be much more noticeable and therefore in cohorts such as the TCGA where this effect was large this exon was also removed from the analysis.

When the inventors applied the reduced exon set to the TCGA cohort with the additional TCRδ gene segment also removed and comparing the TCRA T cell scores they observed a large difference between the TCGA and TRACERx cohort for tumour samples from both LUAD and LUSC (FIG. 26C: Wilcoxon test, LUAD: P<2.2e−16 LUSC: P<2.2e−16). This difference however is entirely due to GC biases within this reduced exon set. When the GC correction method is used there is only a significant difference in TCRA T cell fraction with LUAD patients, and this only being slightly significant presumably due to cohort population differences (FIG. 26D, Wilcoxon test, LUAD: P=0.028, LUSC: P=0.97).

There were no other biases beyond that which could be fixed by GC correction detected in any other capture kit used in any of the cohorts in this paper. The cohorts using the Nimblegen capture kit were however found to be sufficiently different from that of the exons defined from the Agilent capture kit that all samples from that kit were calculated using exons defined directly from the Nimblegen exon capture regions. There were also some capture kits identified such as Nextera Rapid Capture and IDT xGen Exome Research Panel had extremely low coverage within the TCRA gene. Manually checking the exome capture regions of these kits revealed that no regions within the TCRA gene were included in their design. For any datasets using these kits it is not possible to use TIL ExTRECT to calculate the TCRA T cell fraction.

### Calculation of CDR3 VDJ Scores

The procedure outlined in Levy et al. was followed to calculate the CDR3 VDJ scores. First reads aligning to TCRB (hg19: chr7: 142000817-142510993) and unaligned reads were extracted with samtools, this resulting bam was converted to fastq using bedtools and them the tool IMSEQ (v1.1.0) was used on the resulting output to identify VDJ recombinant reads aligning to the CDR3 region, the number of aligned reads was than normalised by the total number of reads in the original bam file (as measured by samtools flagstat) to create the CDR3 VDJ scores.

### TRACERx100 Patients

The first 100 patients prospectively analysed by the NSCLC TRACERX study (https://clinicaltrials.gov/ct2/show/NCT01888601, approved by an independent research ethics committee, 13/LO/1546) were used in this study. This is identical to the 100 patient cohort originally described in Jamal-Hanjani et al (Jamal-Hanjani et al., 2017). Describing this cohort in brief, informed consent was a mandatory requirement for entry into the TRACERx study. This NSCLC cohort consisted of 68 males and 32 female patients with a median age of 68. Finally the cohort is predominantly made up of early-stage tumours (Ia (26), Ib (36), IIa (13), IIb (11), IIIa (13) and IIIb (1)) and 28 patients also had adjuvant therapy. Both WES (aligned to hg19) and RNA-seq samples were obtained from the TRACERx study for the first 100 patients, the method for processing these samples is as previously described (Jamal-Hanjani et al., 2017). Notably for the WES samples, exome capture was performed using a custom version of Agilent Human All Exome V5 kit as per the manufacturer instructions.

### TCGA LUAD and LUSC Cohorts

Aligned BAM files (hg38) from the TCGA LUAD and LUSC cohorts were downloaded from the genomic data commons (dataset ID: phs000178.v10.p8). Sample purity and ploidy calls were generated using ASCAT (v2.4.2), and obtained from a previous analysis of the TCGA data (Middleton et al., 2020). In short, Affymetrix SNP6 profiles from paired tumour-normal samples (dataset ID: phs000178.v10.p8) were processed by PennCNV libraries (K. Wang et al., 2007) to obtain BAFs and log ratios which were GC corrected before being processed with ASCAT. Immune related data including leukocyte fraction and CD8+ fraction was acquired from Thorsson et al. 2018.

### Cancer Cell Line Data

The non-T cell derived cell lines HCT116 were sequenced with Illumina HiSeq 2500 and aligned with bwa mem using hg19 as described in López et al., 2020. The T cell derived cell lines were from the dataset described in Ghandi et al. 2019 downloaded from the Sequence Read Archive (SRA) under accession number PRJNA523380. Cell lines derived from T cells were chosen ensuring that any cell line derived from precursor T cell acute lymphoblastic leukemia was excluded as these have not undergone VDJ recombination. This process led to WES data from three cell lines being chosen: JURKAT, HPB-ALL and PEER.

Due to the difficulty of running ASCAT without matching germline samples, for all cell line work the naïve TCRA T cell fraction was used.

### Orthogonal Immune Measures: 1. RNA-Seq Signatures

The method of Danaher et al. (Danaher et al., 2018) was used as the primary method of estimating T cell content from RNA-seq measures as it has been previously demonstrated that this is most strongly correlated to TIL scores calculated in TRACERx (Rosenthal et al., 2019). Other RNA-seq signature tested against the TCRA T cell fractions were the Davoli method (Davoli, Uno, Wooten, & Elledge, 2017), xCell (Aran, Hu, & Butte, 2017), TIMER (Li et al., 2017) and EPIC (Racle, de Jonge, Baumgaertner, Speiser, & Gfeller, 2017).

### Orthogonal Immune Measures: 2. Pathology TIL Scores

TILs were estimated, as previously described in Rosenthal et al. (Rosenthal et al., 2019), from histopathology slides using internationally established developed the guidelines, by International Immuno-Oncology Biomarker Working Group (Hendry et al., 2017). In brief, the relative proportion of stromal area to tumour area was determined from the pathology slide of a given tumour region. TILs were reported for the stromal compartment (=percent stromal TILs). The denominator used to determine the percent stromal TILs was the area of stromal tissue (that is, the area occupied by mononuclear inflammatory cells over total intratumoral stromal area) rather than the number of stromal cells (that is, the fraction of total stromal nuclei that represent mononuclear inflammatory cell nuclei). This method has been demonstrated to be reproducible among trained pathologists (Denkert et al., 2016). An inter-person concordance was performed, and this demonstrated high reproducibility. The International Immuno-Oncology Biomarker Working Group has developed a freely available training tool to train pathologists for optimal TIL assessment on haematoxylin-eosin slides (www.tilsincancer.org).

### Example 3—Assessment of the Prognostic Value of the WES Derived TCRA T Cell Fractions in the TRACERx100 and TCGA NSCLC Cohorts

### Introduction

To explore the potential clinical utility of TIL ExTRECT, the inventors first considered whether the TCRA T cell fraction was prognostic in the TRACERx100 NSCLC cohort. TIL levels inferred from histopathological H&E slides from samples in the TRACERx100 cohort have previously been associated with disease-free survival (AbdulJabbar et al., 2020). The inventors therefore explored whether they could identify a similar association using the novel TCRA T cell fractions described herein. A similar investigation was also performed using data from the TCGA.

Finally, the inventors used their approach to quantify the TCRA T cell fraction in matched blood samples in these cohorts.

### Results

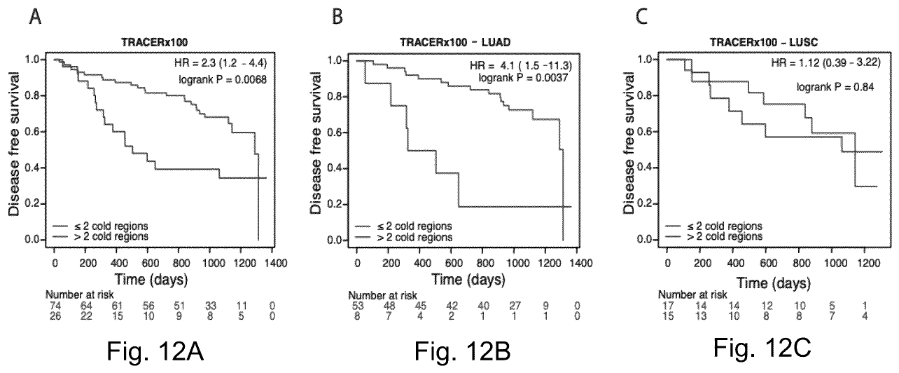

Dividing the patients in the TRACERx100 NSCLC cohort into two groups according to the number of immune cold regions within a patient's tumour (22 immune cold regions, where an immune cold region is defined as one with a TCRA T cell fraction≤0.1), revealed a significant difference: the observed presence of two or more immune cold regions was found to be associated with reduced relapse-free survival (see FIG. 12A, log rank test P=0.0068, HR=2.3). The threshold of ≥2 immune cold regions was chosen based on expert knowledge (see e.g. AbdulJabbar et al., 2020). Separating the cohort by histology revealed that this significance was primarily derived from LUAD patients, while there was no significance in relapse-free survival in LUSC patients (see FIG. 12B, LUAD: P=0.0037, HR=4.1; FIG. 12C, LUSC: P=ns, HR=1.12).

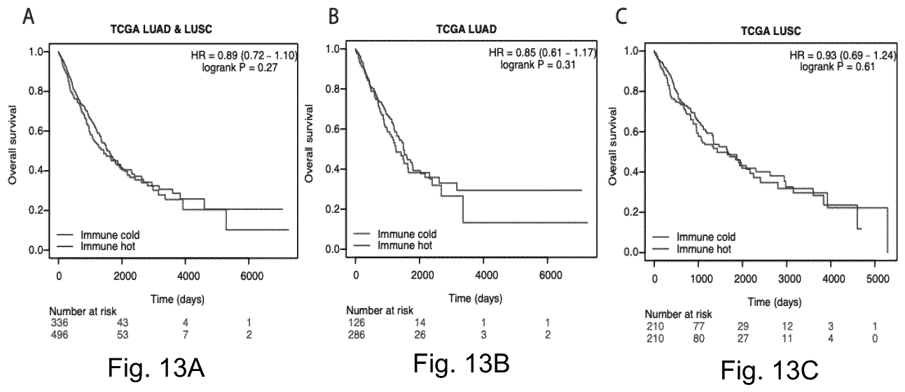

This result in the TRACERx100 cohort suggests the TCRA T cell fraction has significant associations with relapse-free survival for LUAD patients. However, when the inventors assessed whether overall survival in the TCGA LUAD dataset (Table 2) also associated with TCRA T cell fraction they observed no significant relationship (see FIG. 13, log rank test, P=ns) when dividing the patients into two categories based on the definition of an immune cold region having a TCRA T cell fraction <0.1. The TCGA dataset is single region and suffers from sampling bias and cannot distinguish patients who have tumours that are uniformly cold vs only a single cold region. The lack of significance within the TCGA cohort compared to the TRACERx100 suggests the importance of multi-region immune data when predicting survival.

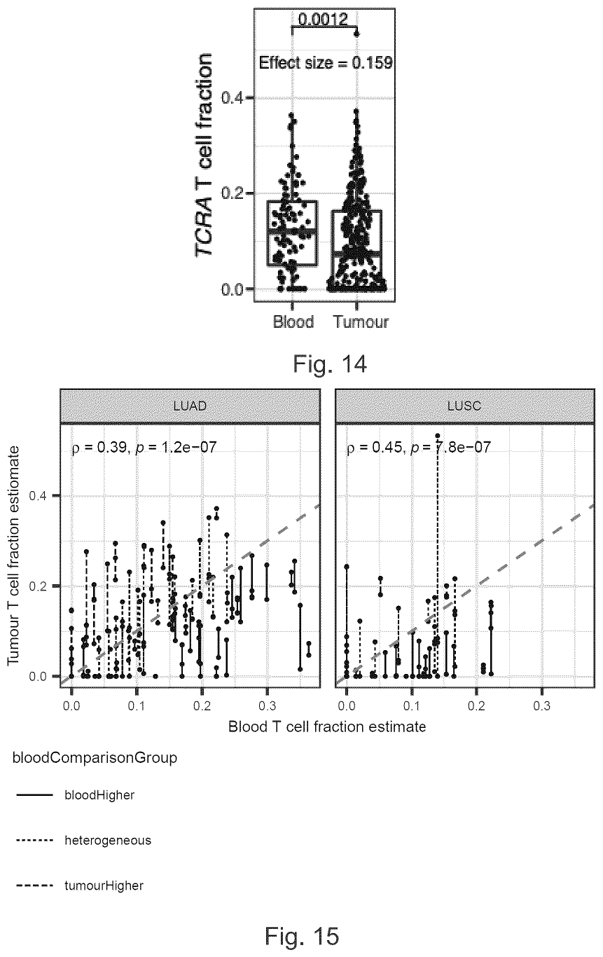

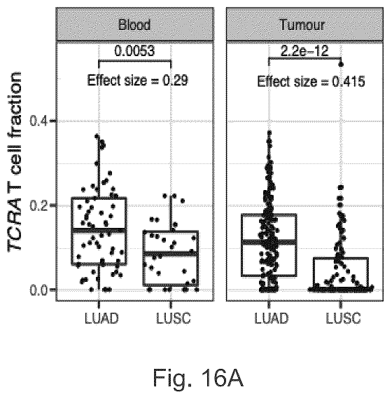

In addition to estimating the TCRA T cell infiltrate in tumour samples, TIL ExTRECT can also be applied to matched blood samples from the TRACERx100 cohort. The inventors observed that matched blood samples had a significantly higher TCRA T cell fraction (see FIG. 14, wilcoxon test P=0.0012, effect size=0.16) than their corresponding paired primary tumour samples. However, it is worth noting that this proportion reflects the proportion of cells with DNA, thus it is likely that if red blood cells were accounted for this proportion of T cells in blood would be much lower. Notably, the tumour TCRA T cell fraction was not consistently lower in tumour regions compared to matched blood. Indeed, a substantial minority (139/339) of tumour regions harboured higher TCRA T cell fractions within the tumour region than in the matched blood. Summarising this on a patient rather than region level, 44 patients had all regions with lower scores than the matched blood sample, 24 patients had all regions with higher scores than the matched blood sample and the remaining 32 were heterogeneous. The inventors also observed (using the TRACERx100 data, where one blood sample is matched to multiple tumour regions due to the multi-region nature of the data) that tumour TCRA T cell fractions were significantly associated with blood TCRA T cell fraction in both LUAD and LUSC (LUAD: ρ=0.39, P=1.2e−07, LUSC: ρ=0.45, P=7.8e-07, see FIG. 15) suggesting the TCRA T cell fractions in the blood and the tumour from the same patient are associated.

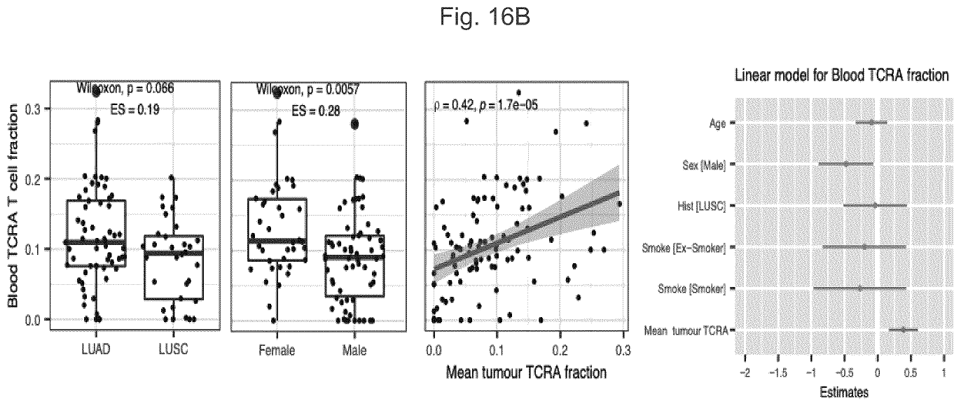

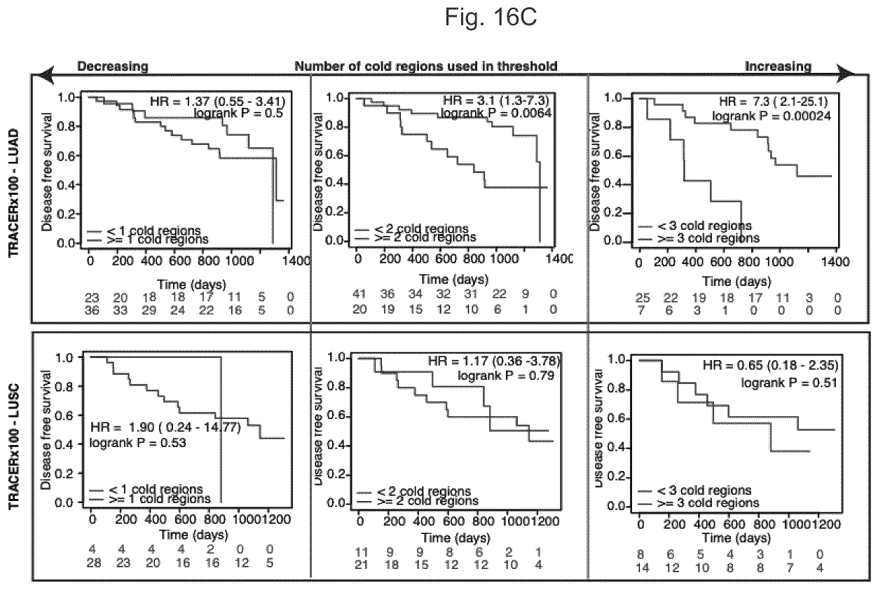

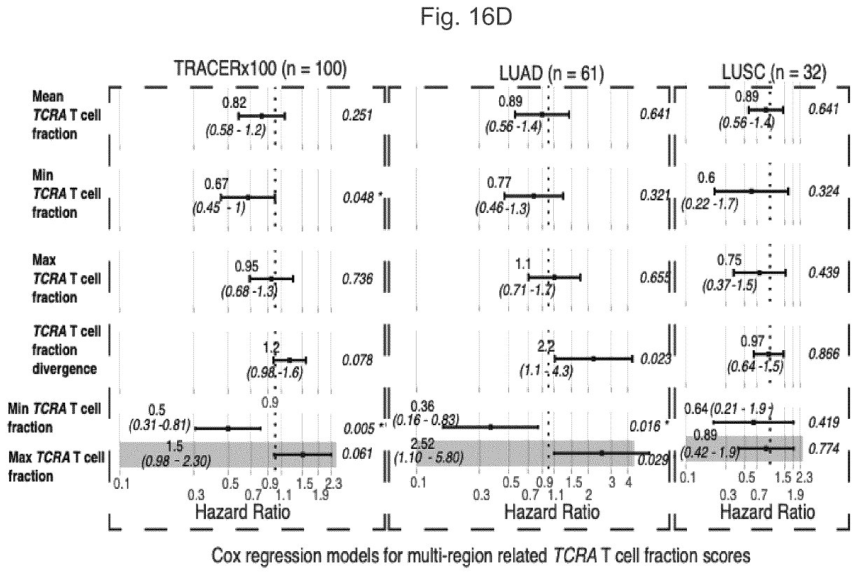

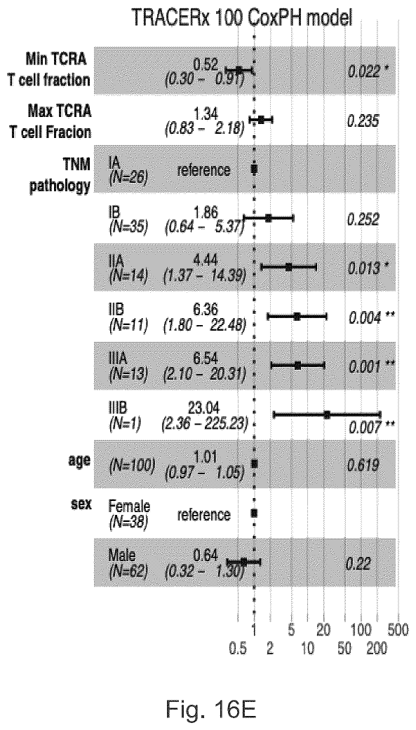

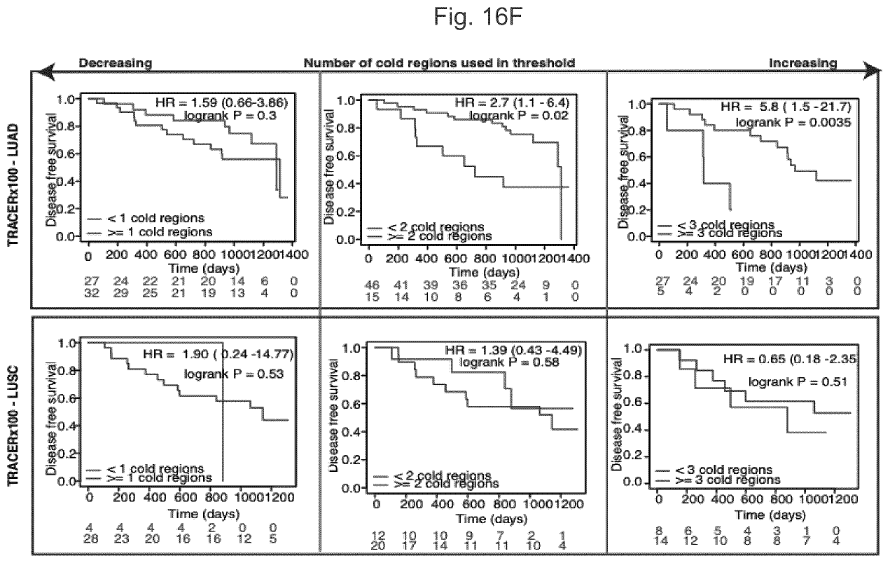

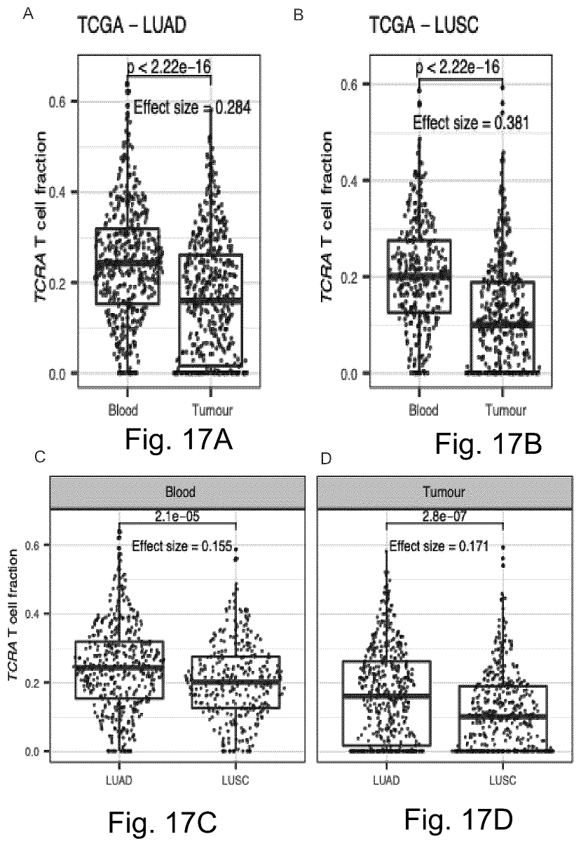

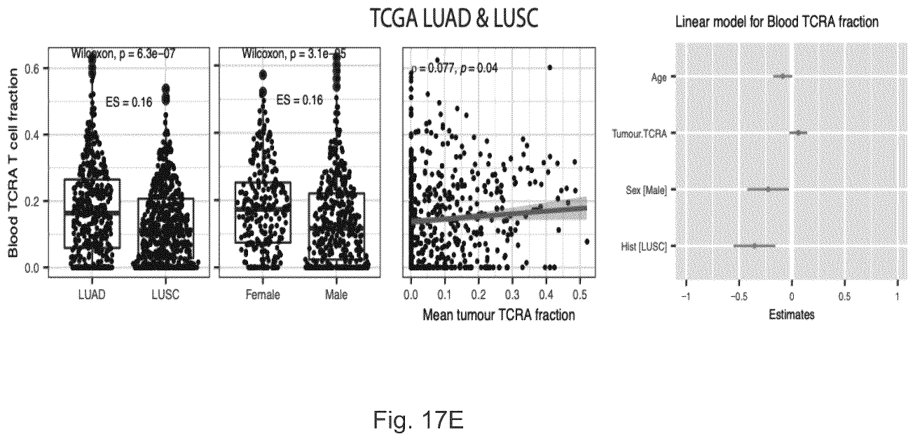

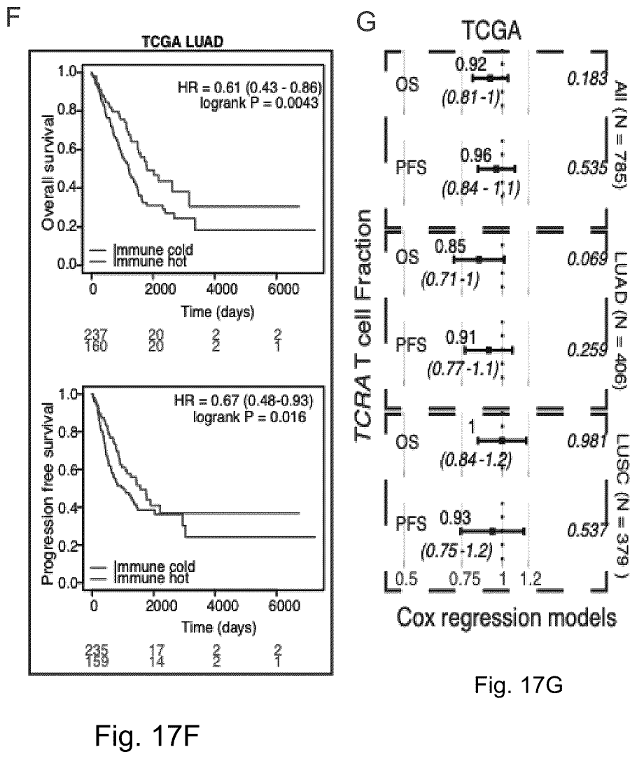

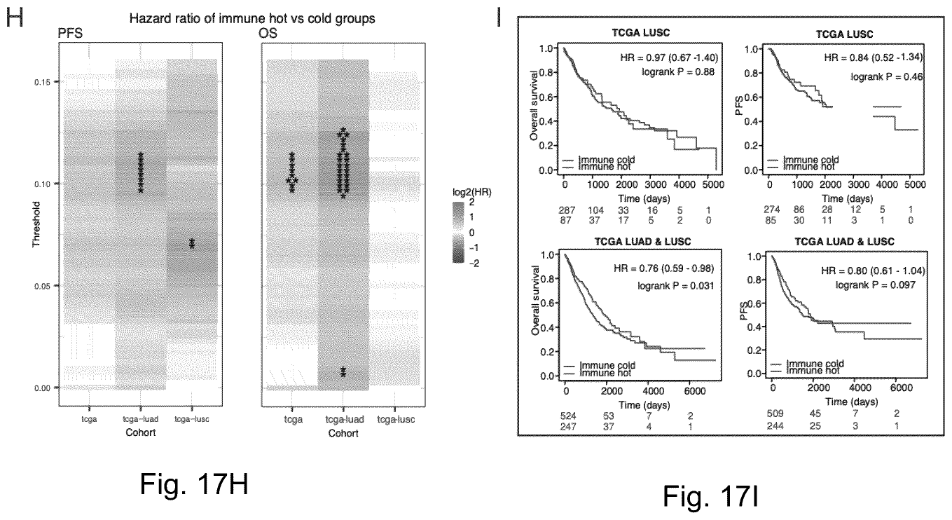

Splitting the TRACERx100 cohort by histology the inventors found that the blood TCRA T cell fraction was significantly higher in LUAD patients compared to LUSC (see FIG. 16A, left plot, Wilcoxon test P =0.0053, effect size=0.29), primary tumour samples also had higher TCRA T cell fraction identified in LUAD compared to LUSC tumours (see FIG. 16A, right plot, Wilcoxon test, P=2.2e−12, effect size=0.42). Similar results were observed in TCGA tumours (see FIGS. 17A-D), where the inventors also found significant differences according to sample type with the TCRA T cell fraction found in blood being higher than the primary tumour (see FIGS. 17A and B, Wilcoxon test LUAD: P<2.2 e−16, effect size=0.28, LUSC: P<2.2 e−16, effect size =0.38). These results suggest that a tumour which stimulates a strong immune response may also lead to higher T cell levels circulating in the blood or potentially that higher T cell levels in the blood may allow a stronger immune response in the tumour to take place.

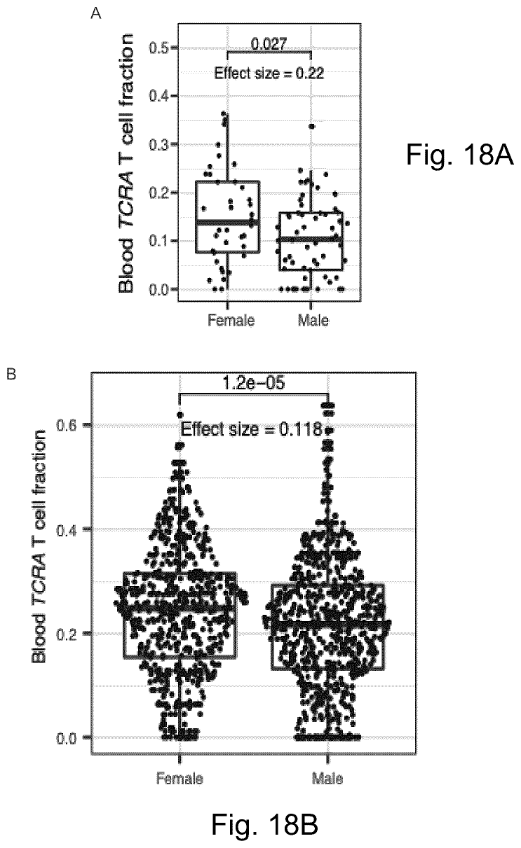

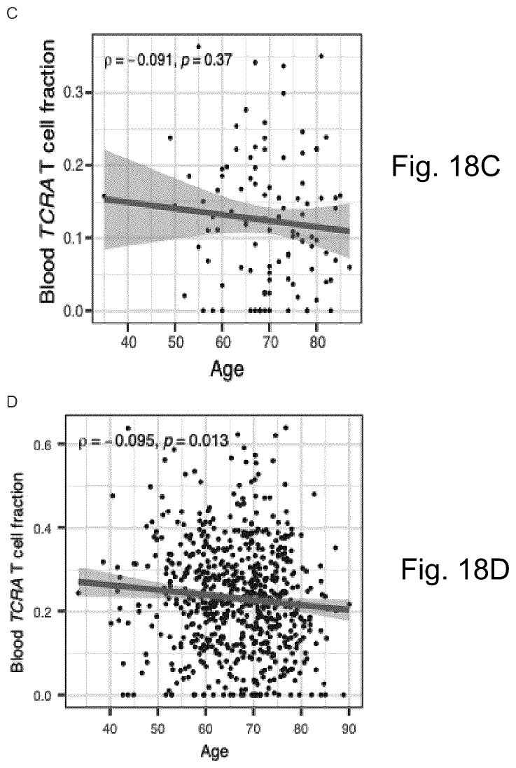

To further investigate factors that could explain the variation in TCRA T cell f fraction within the blood samples, the inventors considered the clinical characteristics of patients, including their sex, and age. Indeed, it has been noted that there is considerable sexual dimorphism in the human immune system during ageing (Márquez et al., 2020) with decreased CD8+ T cell content being associated both with increasing age and the male sex. For both the TRACERx100 and TCGA cohorts there was a significant difference between male and female patients in terms of the TCRA T cell fraction in blood samples, with increased levels found in females (TRACERx100: FIG. 18A, Wilcoxon test P=0.027, effect size=0.22. TCGA: FIG. 18B, Wilcoxon test, P=1.2e−5, effect size=0.12). The effect of age was weaker; the TRACERx100 cohort showed a slight negative trend that was not significant (see FIG. 18C: ρ=−0.091, P=0.37), while the TCGA cohort revealed a weak but significant association (see FIG. 18D: ρ=−0.095, P=0.013).

In a further analysis, the inventors analysed LUAD and LUSC separately and separated patients into either immune hot and cold groups based on the number of cold regions present in the tumour. Here, they used a simple threshold based on the mean TCRA T cell fraction in all tumour regions (0.081) and restricted their analysis to tumours with greater than the number of cold regions used in each threshold. The results in FIG. 16C show that for LUAD as the number of cold regions used in the threshold is increased there is greater significance in survival between the immune hot and cold groups (LUAD: >=2 cold regions, HR=3.1, P=0.0063 logrank test, LUAD: >=3 cold regions HR=7.3, P=0.00024 logrank test). In contrast for LUSC there was no significant difference in survival between the immune cold or hot groups for any of the different thresholds (FIG. 16C). Using the median (0.074) instead of the mean as a threshold for immune hot or cold regions yielded similar results (FIG. 16F).

In the single region TCGA LUAD cohort the inventors also found a significance in survival between hot and cold tumours (overall survival (OS): HR=0.61, P=0.0043, progression free survival (PFS): HR=0.67 P=0.016-see FIG. 17F) using the mean as the threshold (0.11) between hot and cold tumours. For both OS and PFS there were a range of possible thresholds that would yield similar results (FIG. 17H). Using the same threshold for defining immune hot and cold tumours there were no significant associations with survival for either OS or PFS in the TCGA LUSC cohort (FIG. 17I).

The inventors then used Cox regression models to ascertain if a continuous analysis using different multi-region scores related to the TCRA T cell fraction was equally prognostic for survival. The inventors chose to study the following four scores for each patient: 1) the mean TCRA T cell fraction across all regions, an indicator of the overall T cell infiltration in the tumour, 2) the minimum TCRA T cell fraction across all regions 3) the maximum TCRA T cell fraction across all regions, and 4) the TCRA T cell fraction divergence defined as the maximum region TCRA T cell fraction divided by the upper 95% confidence interval (to avoid division by 0 or very small numbers) of the minimum region TCRA T cell fraction. The latter score represents the fold difference between the region with the maximum and minimum TCRA T cell fraction and when high may indicate T cell divergence/heterogeneity, perhaps indicative of a subclone having undergone immune escape. Since the relative region immune escape score is determined by the minimum and maximum score the inventors also built a Cox model including both these scores. The results of this analysis are summarised in FIGS. 16D and 17G. Consistent with the importance of the tumour region with the lowest TCRA score³², the minimum TCRA fraction across tumour regions was prognostic in the entire TRACERX cohort (HR=0.52, P=0.048). However, both the mean and maximum TCRA T cell fraction were not significant in any group. The TCRA T cell fraction divergence score was significant in LUAD (HR=2.2, P=0.023 logrank test). A model containing both the minimum and maximum TCRA scores were significant in both the entire cohort (Min: HR=0.5, P =0.005, Max: HR=1.5, P=0.061), and the LUAD subset (Min: HR=0.36, P=0.016, Max: HR=2.52, P=0.029), see FIG. 16D, suggesting that there is added predictive potential when considering the heterogeneity of the TCRA T cell fraction. No TCRA related score was significant in LUSC and the maximum TCRA T cell fraction was only significant in combination with the minimum and only in LUAD.

When combining the minimum and maximum TCRA T cell fraction with other clinical phenotypes such as tumour stage, sex and age, the minimum TCRA T cell fraction was still significant (HR=0.52, P=0.022, see FIG. 16E). Examining the single region TCGA survival data for both overall survival (OS) and progression free survival (PFS), the TCRA T cell fraction was not significant in a univariate Cox model for any comparison, the closest association being in TCGA LUAD in the OS model (HR=0.85, P=0.069).

Combined, the inventors believe this data to be suggestive that TCRA T cell is prognostic for survival in LUAD patients. Particularly the inventors noted that a stronger overall signal was seen in the TRACERx100 cohort despite its smaller size, indicating the importance of immune heterogeneity within a tumour that is revealed by multiregion data.

### Methods

See Examples 1-2.

### Multi-Sample Tumour Cohort of Patients

The multi-sample pan-cancer cohort (see Table 5) was created by combining the TRACERx cohort with a subset of the cohort presented recently by Watkins et al. Tumours were included if they had at least two regions sequenced in the primary tumour for which it was possible to calculate the TCRA T cell fraction using TIL ExTRECT. The final cohort therefore consisted of a multi-region primary tumour data set with the addition of any metastasis samples that were also sequenced for these patients.

TABLE 5
Overview of patients in the multi-sample pan-cancer cohort

| | | Number of | Number of |
|---|---|---|---|
| Study | Histology | patients | regions |
| TRACER × 100 | LUAD | 60 | 166 |
| TRACER × 100 | LUSC | 32 | 106 |
| TRACER × 100 | Lung (other) | 7 | 26 |
| Brastianos et al. | Lung carcinoma | 1 | 3 |
| Gerlinger et al. | KIRC | 10 | 66 |
| Harbst et al. | SKCM | 3 | 17 |
| Lamy et al. | BLCA | 22 | 52 |
| Savas et al. | BRCA ER+ | 3 | 36 |
| Murugaesu et al. | ESCA | 8 | 37 |
| Suzuki et al. | Glioma | 12 | 45 |
| Turajlic et al. | KCCS | 1 | 3 |
| Turajlic et al. | KIRC | 14 | 101 |
| Turajlic et al. | KIRP | 1 | 13 |
| Messaoudene et al. | BRCA ER+ | 3 | 32 |
| Messaoudene et al. | BRCA HER2+ | 5 | 44 |

Besides TRACERx100 the following datasets were combined into the final multi-sample pan-cancer cohort:
1. Brastianos et al.—a cohort focused on studying brain metastasis originating from different histologies, only tumours with multi-region primary samples from this cohort were included.
2. Gerlinger et al.—A multi-sample primary cohort of kidney renal clear cell carcinoma (KIRC) patients.
3. Harbst et al.—A multi-region primary cohort of skin cutaneous melanoma (SKCM) patients.
4. Lamy et al.—A multi-region primary cohort of bladder cancer patients (BLCA)
5. Savas et al.—A multi-sample cohort of ER+ and triple negative breast cancer patients (BRCA ER+ and TNBC)
6. Suzuki et al.—A multi-region primary cohort of glioma.
7. Turajlic et al.—A multi-region primary cohort of kidney clear cell renal cell carcinoma (KIRC), Kidney renal papillary cell carcinoma (KIRP) and Kidney Chromophobe (KICH) patients.
8. Messaoudene et al.—A multi-region primary cohort of HER2+ and ER+ breast cancer patients.

### Selection of Subregions for Multi-Region Sequencing in Different Data Sets

In all of the multi-region cohorts regions were selected though by different methods (see associated publications) with two main criteria in mind, first that tumour content be maximised at the expense of stromal in order to assure good quality mutation and copy number analysis for the main goal of the genomic analysis and second that each region represent a physically separate and distinct part of the tumour. In cases where these were not at separate sites different measures were used. In the TRACERx100 cohort for example regions sequenced were a minimum of 3 mm apart.

### Example 4—TCRA T Cell Fraction is Predictive of Response to Immune Checkpoint Blockade

### Introduction

To further explore where TCRA T cell fraction may be of clinical utility, the inventors evaluated whether it could be exploited to predict response to immune checkpoint blockade, using data from the CPI1000+ cohort (Litchfield et al, 2020).

### Results

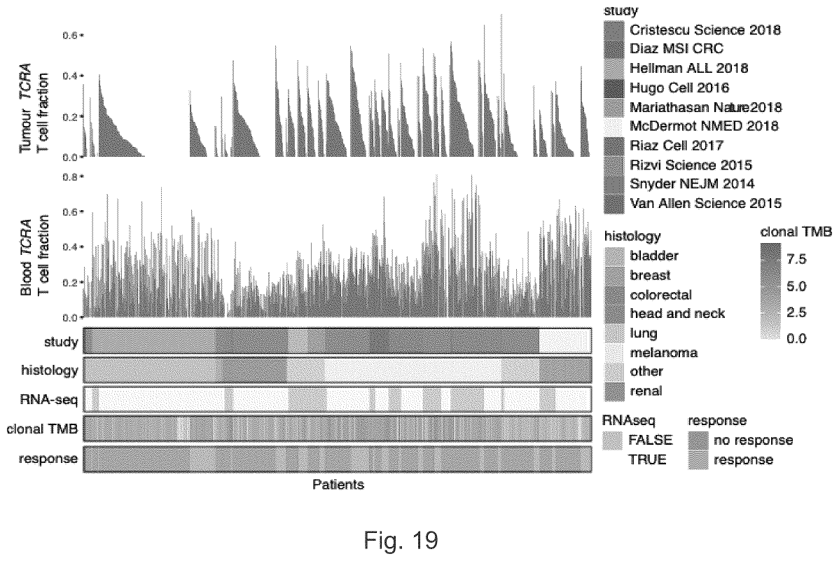

The CPI1000+ cohort (Litchfield et al, 2020) multi-study dataset consists of 1070 CPI (checkpoint inhibitor) treated tumours receiving either anti-CTL4 or anti-PDL1 therapy across 8 main cancer types (see FIG. 19 and Table 3). Response was defined as a patient having a radiological response with complete response (CR) or partial response (PR), while a non-responder was defined as stable disease (SD) or progressive disease (PD) by RECIST criteria. Table 3 details the percentage of tumours with both WES and RNA-seq data.

TABLE 3
Overview of samples in the CPI1000+ cohort excluding Snyder et al., 2017

| CPI1000+ | Melanoma | Bladder | Head and neck | Renal | NSCLC | Other | Colorectal | Breast | Total |
|---|---|---|---|---|---|---|---|---|---|
| WES | 350 | 261 | 107 | 104 | 76 | 76 | 20 | 14 | 1008 |
| | | | | | | | | | (89%) |
| RNA-seq | 215 | 348 | 105 | 72 | 4 | 70 | 5 | 14 | 833 |
| | | | | | | | | | (74%) |
| WES + | 209 | 247 | 105 | 72 | 4 | 70 | 5 | 14 | 726 |
| RNA-seq | | | | | | | | | (65%) |
| Patients | 364 | 363 | 107 | 105 | 76 | 76 | 20 | 14 | 1125 |

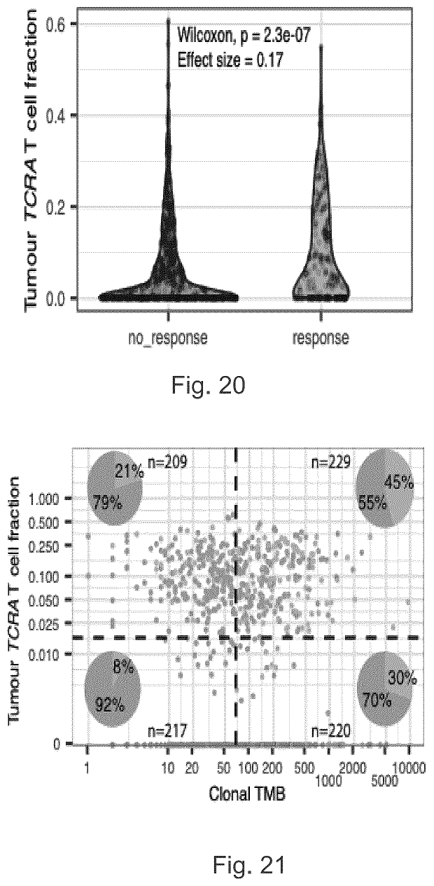

Consistent with the importance of the tumour TCRA T cell fraction in predicting response to CPI, the inventors observed a significant difference in TCRA T cell fractions between responders and non-responders across the entire cohort (see FIG. 20, P=2.3e−7, effect size=0.17). The median TCRA T cell fraction in the tumour DNA sample for responders was 0.053 (Q1=0, Q3=0.17) compared to 0.000268 (Q1 =0, Q3=0.084) in non-responders. With regards to immune cold tumours (defined here as tumours with TCRA T cell fraction <0.1), there was a highly significant enrichment in the non-responders (FIG. 21, immune cold tumours are those below the dotted line, 78% vs. 63%, Fisher's exact test, odds ratio (OR)=0.47, P=2.25e−06).

Separating the cohort by both mutation burden (high: total clonal TMB ≥68, low: clonal TMB<68) and the immune microenvironment (hot: TCRA T cell fraction ≥0.016, cold: TCRA T cell fraction <0.016) reveals that the association between TCRA T cell fraction and response is independent of mutation burden (see FIG. 21). In the low mutation burden patients, there is a 21% response rate in immune-hot tumours compared to 8% in immune cold. In the high mutational burden tumours there is a 45% response rate when the tumour is immune hot and 30% when it is cold.

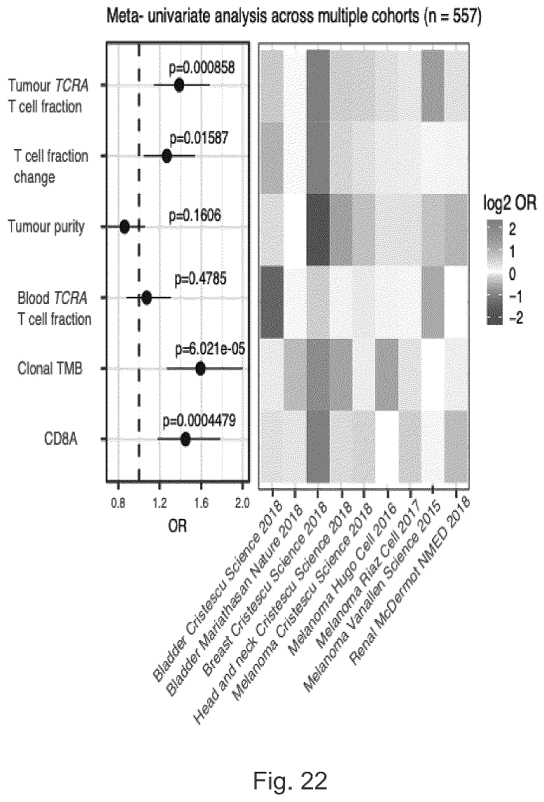

To further evaluate the utility of the TCRA T cell fraction in comparison to RNA-seq based measurements, all studies with greater than 10 samples deriving from an individual cancer type with both RNA-seq and TCRA T cell fractions were selected for a univariate meta-analysis (see FIG. 22: 557 patients across 7 studies and 5 cancer types). As expected, TCRA T cell fraction (odds ratio (OR)=1.39, P =0.00858), clonal TMB (OR=1.59, P=6.021e−05) and CD8A expression (OR=1.45, P=0.0004479) were all found to be significantly associated with response. Neither the TCRA T cell fraction from the blood nor tumour purity was found to be significantly associated with response, while the difference between tumour and blood TCRA T cell fraction was marginally significant (OR=1.27, P=0.016).

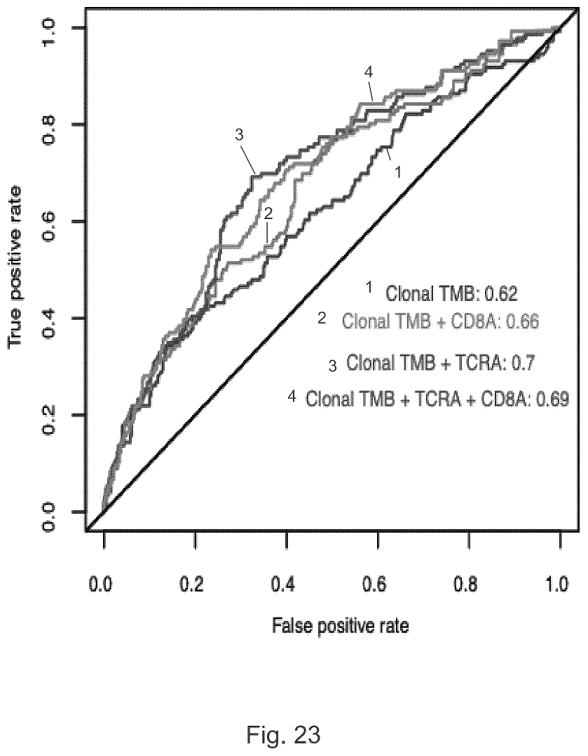

The inventors then assessed whether the tumour TCRA T cell fraction provided additional clinical utility beyond TMB (tumour mutational burden), and moreover, whether it improved prediction of response to a greater extent than RNA-seq measures such as CD8A expression. They created four general linear models (GLM) to predict whether a patient was a responder or non-responder. The first model consisted of only clonal TMB, while the second and third used clonal TMB combined with either CD8A RNA-seq expression or the TCRA T cell fraction. The fourth used clonal TMB combined with both CD8A expression and the TCRA T cell fraction. Either using CD8A expression or TCRA improved the predictive value of the model (see FIG. 23 AUC=0.66 for CD8A expression and AUC=0.70 for TCRA T cell fraction compared to 0.62 for clonal TMB alone). However out of these only the model for clonal TMB+TCRA T cell fraction (ROC test, P=0.0028, GLM: clonal TMB+TCRA, AUC=0.68, GLM: clonal TMB, AUC=0.62) was significant compared to clonal TMB alone. Combining the TCRA T cell fraction with CD8A expression failed to significantly increase the predictive value of the model (see FIG. 23, AUC=0.68, ROC test versus Clonal TMB+TCRA model P =0.72). However, examining the significance of the variables in all 4 models, TCRA T cell fraction was more significant than CD8A (GLM: clonal TMB+TCRA, P=4.62e−05; GLM: clonal TMB+CD8A, P=0.000431) and when combined into a single model the TCRA T cell fraction remained significant, but CD8A expression did not (TCRA, P=0.00601, CD8A, P =0.06246).

Taken together, these results suggest the tumour TCRA T cell fraction can be used as a substitute for RNA-seq measures of CD8+ infiltrate, and, moreover, the WES based TCRA T cell fraction estimation adds prognostic value to TMB estimates.

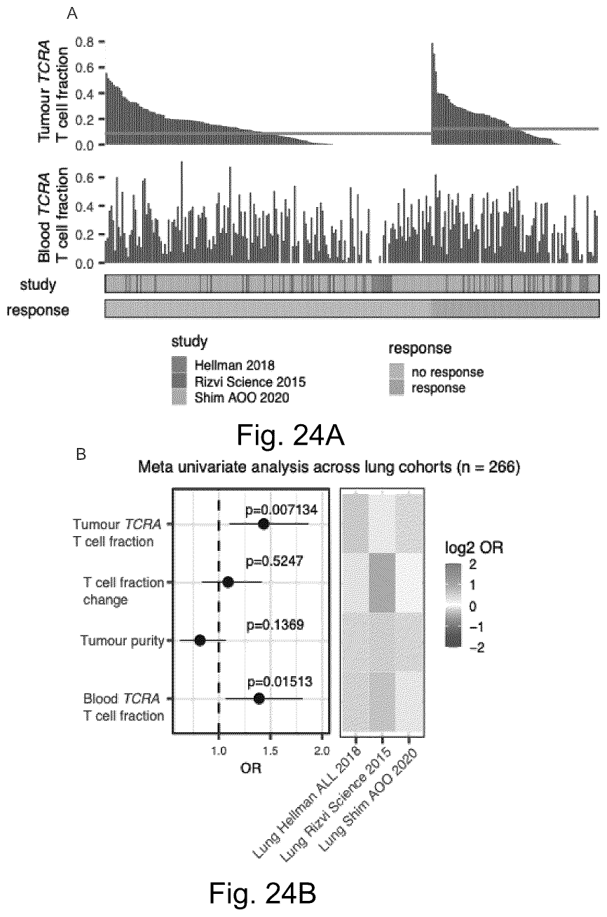

Given that RNA-seq is unavailable in many cases the inventors next assessed the predictive potential of the TCRA T cell fraction in a combined NSCLC CPI cohort (see Table 4). FIG. 24A provides an overview of this cohort which contains 266 patients assayed with WES and, crucially, lacks any RNA-seq or other orthogonal immune measures. Analysis of this lung CPI cohort, running a univariate analysis (see FIG. 24B) TCRA T cell fraction (OR=1.44, P=0.005) and blood TCRA T cell fraction (OR=1.39, P=0.0015) were found to be significantly associated with response to CPI. On an individual study basis TCRA T cell fraction had OR >1 in both the Hellman and Shim cohorts but not the Rizvi, in contrast blood TCRA had OR >1 in all three separate studies. These results show that a TCRA T cell fraction can be calculated from WES data alone and that such estimates from NSCLC as well as from matched blood are predictive of response to immunotherapy.

TABLE 4
Overview of patients
in the CPI Lung cohort.
Study (Number of patients)

| Rizvi | Hellman | Shim | Total |
|---|---|---|---|
| et al. | et al. | et al. (195) | (266) |
| (34) | (37) | | |

### Methods

See Examples 1-2.

### CPI1000+ Meta-Analysis of Cohorts

The CPI1000+ cohort is fully described in Litchfield et al. (2020) and contains the following datasets: 1. Snyder et al. (Snyder et al., 2014), an advanced melanoma anti-CTLA-4 treated cohort. 2. Van Allen et al. (Van Allen et al., 2016), an advanced melanoma anti-CTLA-4 treated cohort. 3. Hugo et al. (Hugo et al., 2016), an advanced melanoma anti-PD-1 treated cohort.4. Riaz et al. (Riaz et al., an advanced melanoma anti-PD-1 treated cohort. 5.2017),
Cristescu et al. (Cristescu et al., 2018) an advanced melanoma anti-PD-1 treated cohort. 6. Cristescu et al. (Cristescu et al., 2018) an advanced head and neck cancer anti-PD-1 treated cohort. 7.
Cristescu et al. (Cristescu et al., 2018) “all other tumor types” cohort (from KEYNOTE-028 and KEYNOTE-012 studies), treated with anti-(Snyder et al., 2017), a metastatic PD-1.8. Snyder et al. urothelial cancer anti-PD-L1 treated cohort. 9. Mariathasan et al. (Mariathasan et al., 2018), a metastatic urothelial cancer anti-PD-L1 treated cohort. 10. McDermot et al. (McDermott et al., 2018), a metastatic renal cell carcinoma anti-PD-L1 treated cohort. 11.
Rizvi et al. (Rizvi et al., 2015), a non-small cell lung cancer anti-PD-1 treated cohort. 12. Hellman et al., a cohort of non-small cell lung cancer samples treated with anti-PD-1 used by Litchfield et al., 2020.13. Le et al. (Le et al., 2015), a colorectal cancer cohort treated with anti-PD-1 therapy.

Of these studies Snyder et al. (Snyder et al., 2017) was excluded from the analysis due to extremely poor coverage within the TCRA gene. All samples were aligned to hg19 using bwa mem (v0.7.15) with purity and copy number data was calculated from ASCAT as described in Litchfield et al. (2020). Notably, 1008/1125 (90%) samples had WES data, 941/1125 (83%) had sufficient purity and coverage to enable copy number calculation enabling the TCRA T cell fractions to be calculated. 833/1125 (74%) of these samples had matched RNA-seq data allowing orthogonal assessment of T cell estimates. For an extension to this dataset, Shim et al. (Shim et al., 2020) a NSCLC anti-PD-1 treated cohort was added for a specific NSCLC analysis.

### Univariate and Multivariate Model for CPI Response

For the univariate model an adapted procedure from Litchfield et al. (2020) was followed with the main difference being that only samples with complete data (RNA-seq for CD8A, clonal TMB and TCRA T cell fraction) were included. The univariate model meta-analysis was conducted using R package ‘meta’ (version 4.13-0). The multivariate model was created with general linear models using the function ‘glm’ from the ‘stats’ R package using default values. The R package ‘ROCR’ (version 1.0-11) was used for the ROC curve analysis.

### Example 5—Determinants of the TCRA T Cell Fraction in Blood, Normal and Malignant Tissue

While previous analyses have necessarily focussed on T cell infiltrate in tumour tissue, the TIL ExTRECT method described herein provides the opportunity to explore T cell infiltrate in any sample which has been subject to WES. In this example, the inventors therefore undertook an analysis to evaluate the key determinants of T cell immune infiltrate in different sample types.

### Results

The inventors first explored the extent of T cell infiltrate within blood taken as a normal control for tumour analysis. Within the TRACERx100 cohort, TCRA T cell fraction in blood was significantly higher in females compared to males and we observed a significant positive relationship between tumour sample TCRA T cell fraction blood TCRA T cell fraction (FIG. 16B, histology: P=0.066, ES=0.19; sex: P=0.0057, ES=0.28; mean tumour infiltration: ρ=0.42, P=1.7e−05). These data suggest that tumour immune infiltrate may influence T cell levels in circulating blood. Sex and mean tumour infiltration were significant univariate predictors of TCRA T cell fraction and remained significant in a linear model predicting blood TCRA T cell fraction controlling for smoking status, age and histology. By contrast, no significant differences in T cell infiltration in blood was observed between LUAD and LUSC patients in the multivariate model.

The inventors also analysed matched blood samples taken from the TCGA LUAD and LUSC cohort and found similar results with sex and tumour histology being strongly associated in a linear model predicting TCRA T cell fraction in blood (FIG. 17E).

The inventors then evaluated T cell ExTRECT in an independent NSCLC dataset with lower coverage from the TCGA, as described above and in Table 2. They observed broadly consistent results in blood samples from LUAD and LUSC TCGA patients (FIG. 17E).

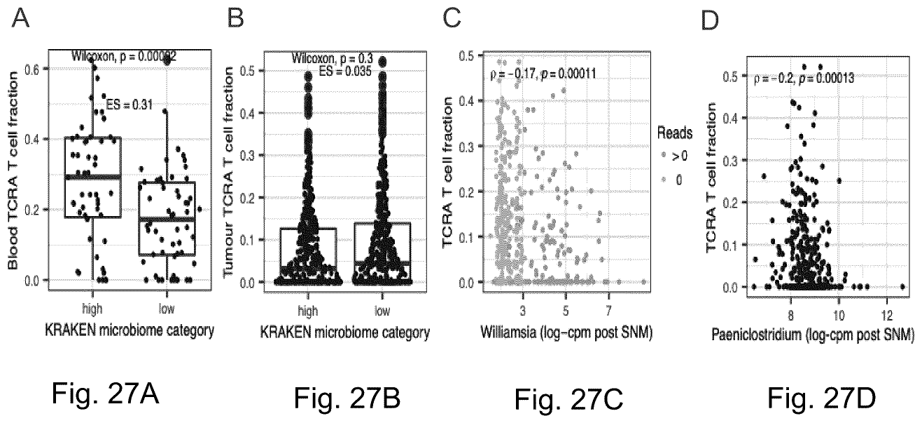

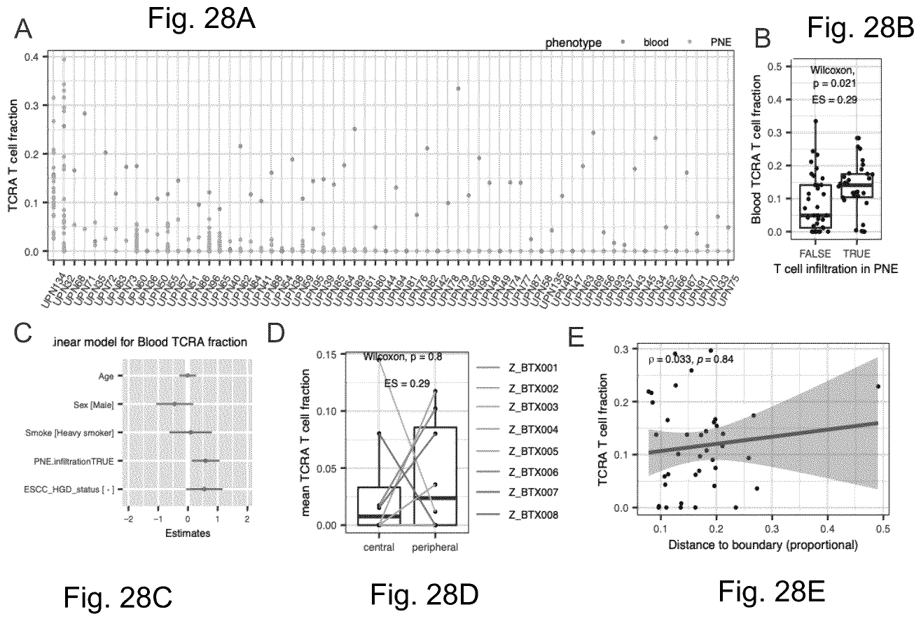

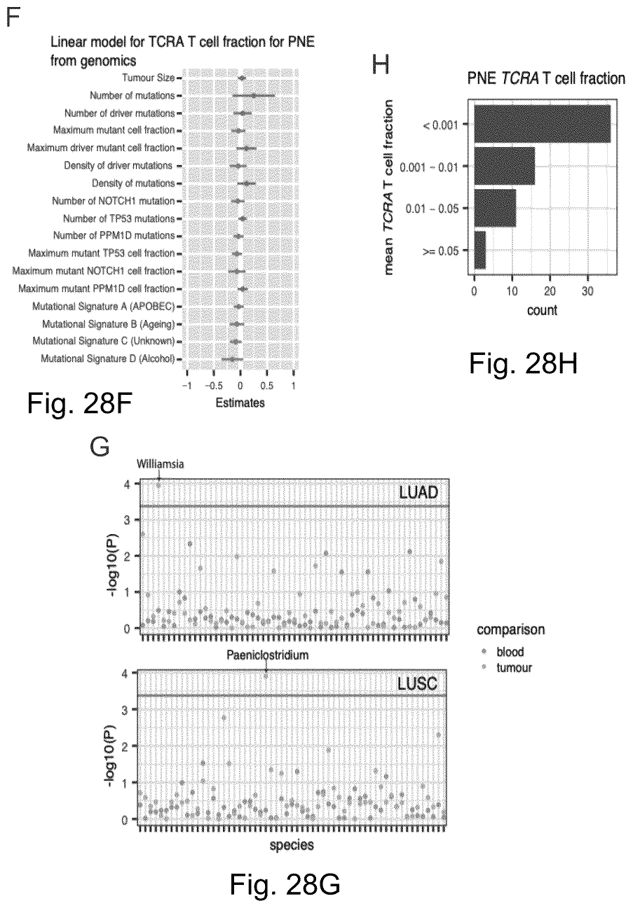

Another factor that would potentially influence T cell infiltration with the blood is the presence of viral or bacterial infections elsewhere in the body. To explore this hypothesis the inventors used the data presented in Poore et al. that used the bioinformatics tool KRAKEN to quantify the number of microbiome reads from WGS blood samples and RNA-seq tumour samples from the LUAD and LUSC TCGA cohort. The inventors observed that blood samples with an elevated number of microbial reads (greater than the median, 6.81) had a significantly higher blood TCRA T cell fraction than the low group (FIG. 27A P=0.00092, ES=0.31, Wilcoxon test). By contrast, no association between total microbial read number and tumour TCRA T cell fraction was observed, suggesting that the presence of viruses or bacteria does not drive the levels of T cell fraction we see in cancers (FIG. 27B P=n.s). The inventors then examined if on an individual species level, there were any viruses or bacteria associated with TCRA T cell fraction (see methods). There were no significant associations after multiple hypothesis adjustment for blood samples. For tumour samples there was one hit each for LUAD and LUSC tumours, for bacteria in genus Williamsia and Paeniclostridium respectively (FIGS. 27C and 28G Williamsia in LUAD: ρ=−0.17, P=0.00011, FDR P=0.013, FIGS. 27D and 28G Paeniclostridium in LUSC: ρ=−0.2, P=0.00013, FDR P=0.015). However, both of these had higher numbers of normalised log-cpm values when TCRA T cell fraction was lower suggesting that the presence of these bacterial species may not lead to increased T cell infiltration and instead they may be opportunistic species taking advantage of the immune cold tumour microenvironment.

To further understand the key determinants of blood TCRA T cell fraction, the inventors explored WES sequencing samples derived from both blood and physiologically normal oesophagus epithelia (PNE) tissue. In particular, the inventors examined a data set containing micro-dissected WES sequencing samples derived from physiologically normal oesophagus epithelia (PNE) tissue, as described by Yokyama et al. Here, there was a wide range of TCRA T cell fraction calculated in the blood samples but the majority of the PNE tissue had no detectable T cell infiltration (FIG. 28A, FIG. 28H). Dividing the participants into those with or without T cell infiltration in the PNE samples revealed a significant association with blood TCRA T cell fraction (FIG. 28B, P=0.021, ES=0.29), again indicating that similarly to tumour samples, high levels of T cell infiltration in normal tissues influences the TCRA T cell fraction visible in the blood. In a linear model predicting T cell fraction in blood, only the infiltration level in normal tissue was significantly independently predictive (FIG. 28C). In a separate linear model, no genomic factors such as normal tissue mutation burden or cancer driver mutation status were found predictive of T cell infiltration in PNE tissue (FIG. 28F). This suggests that the T cell infiltration that is detected is not due to ongoing immune surveillance, and could instead be due to the presence of microbial infections.

To further evaluate the factors influencing T cell infiltrate in tumour tissue, the inventors utilized a recently published pan-cancer cohort of multi-sample data (Watkins et al.), allowing to investigate the extent to which different regions of the same tumour may exhibit disparate levels of immune infiltrate, and whether it was possible to identify a genomic basis for this difference. In total, the inventors were able to evaluate T cell infiltrate in 739 tumour regions from 182 tumours, from 14 cancer types (see Table 5).

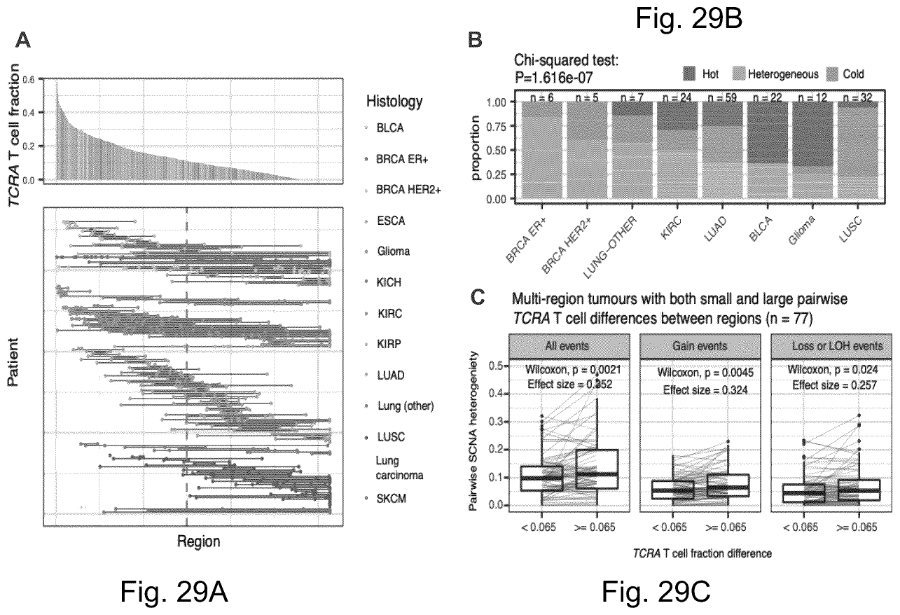

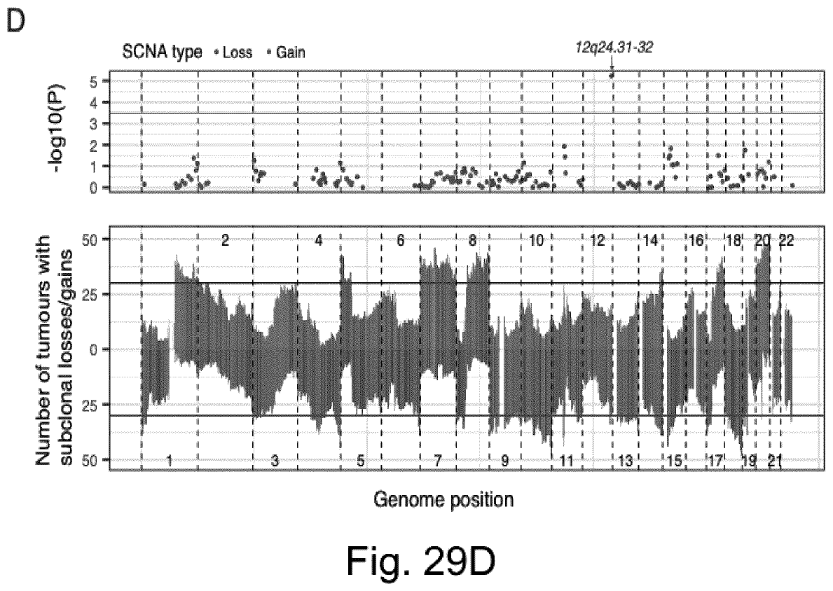

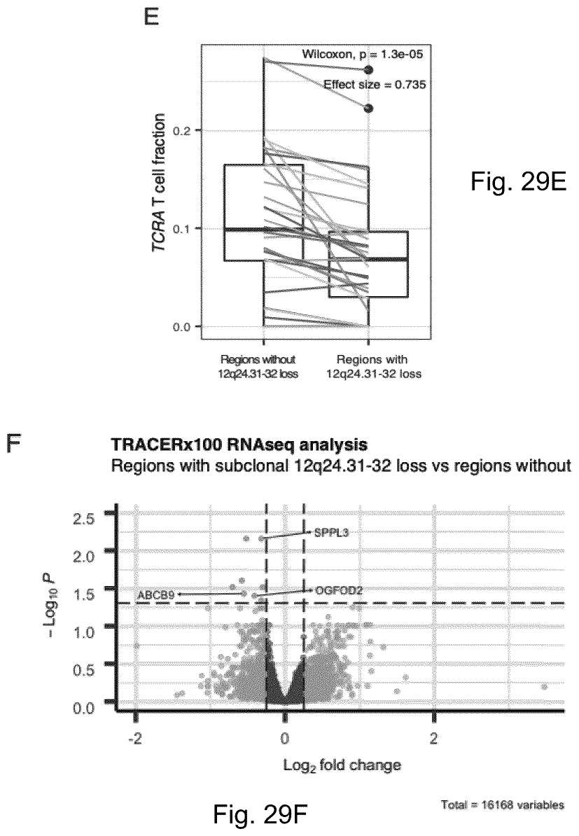

A range of T cell infiltration was observed across the cohort (FIG. 29A, range 0-58%). Interestingly the third highest score with 518 of sequenced cells were found to represent T cells in a single region of a KIRC tumour from patient RMH002. Notably, patient RMH002 had been treated with the anti-angiogenic drug sunitinib for 14 weeks before nephrectomy, and sunitinib treatment has been shown to increase T cell infiltration in KIRC tumours (Haywood et al.) To quantify the extent of T cell infiltrate diversity within tumours, the inventors divided each tumour into one of three categories based on whether the calculated TCRA T cell fraction was uniformly hot across all regions (defined here as having all regions ≥0.11, the mean TCRA T cell fraction in the cohort), whether it was uniformly cold (defined as having all regions <0.11) or whether the T cell fraction was heterogeneous. There was a significant difference in the proportion of uniformly hot, uniformly cold and tumours with heterogeneous immune infiltration assessed by cancer type (FIG. 29B chi-squared test: P =1.62e−07) with BRCA ER+ tumours being the most heterogeneous (83%) and LUSC tumours being the least (22%). There were also clear differences in the extent of immune infiltrate and the prevalence of the different immune group categories uniformly hot, cold or heterogeneous across cancer types. For instance, while BLCA and LUSC had similar numbers of heterogeneous tumours (36% vs 37%), ˜64% of BLCA tumours were uniformly hot and 0% were uniformly cold, while in LUAD 37% tumours were uniformly cold and 25% uniformly hot. This suggests that for certain cancer types, most notably BRCA ER+, BRCA HER+, LUAD, and KIRC, there is a highly localized immune infiltrate, which can be subject to considerable sampling bias.

Next, the inventors examined whether there was any relationship between genomic diversity and immune diversity, investigating subclonal SCNA heterogeneity as a potential mechanism that could lead to a heterogeneous immune response within a single tumour. The inventors first restricted the analysis to tumours with at least three samples and a heterogeneous mixture of T cell fractions (see Methods). Pairwise SCNA heterogeneity between any two regions was calculated as the sum of the proportion of the genome with unique SCNA in either region. FIG. 29C shows with a pairwise analysis that within a tumour pairs of regions with a larger difference in TCRA T cell fraction (>=the mean of all pairwise distances, 0.065) were more likely to have higher levels of pairwise SCNA heterogeneity (All events: P=0.00021, effect size=0.352; gain events: P=0.0045, effect size=0.324; loss or LOH events: P=0.024, effect size=0.257, n=77).

Next, to explore whether any specific subclonal SCNA events were associated with immune depletion or activation, the inventors identified cytoband regions that were subclonally lost or gained in more than 30 tumours in the full pan-cancer multi-sample cohort (FIG. 29D) and tested whether these gain or loss events were associated with changes in TCRA T cell fraction. Only one cytoband-level event, subclonal loss of 12q24.31-32 was found to be significantly associated with a decrease in TCRA T cell fraction (FIG. 29E: P=1.3e−05, effect size=0.735).

To determine whether this effect was due to any single gene the inventors selected the tumours with subclonal loss of 12q24.31-32 within the TRACERx100 cohort and ran a differential gene expression analysis on the associated RNA-seq data. After testing 16, 168 genes only eight remained significant after multiple testing correction: SPPL3, C12orf76, LYRM9, CIT, UBE3B, ABCB9, OGFOD2 and USP30 (FIG. 29F). Notably, SPPL3 was the most significant gene and along with ABCB9 and OGFOD2 is located on 12q24.31. SPPL3 has been recently found to augment B3GNT5 enzyme activity which upregulates cell surface glycosphigolipids that in turn impede class I HLA function and diminish CD8+ T cell activation (Jongsma et al.). Thus, these data suggest that subclonal loss of 12q24.31 may be selected in tumour evolution across cancer types (occurring in 18.7% or 34/182 of tumours within the cohort) as a mechanism of immune evasion.

### Methods

See Examples 1-4.

### KRAKEN TCGA Analysis

Pre-processed microbiome data output from the KRAKEN (Wood & Salzberg) analysis performed by Poore et al. was downloaded from ftp://ftp.microbio.me/pub/cancer_microbiome_analysis/.

To create the high and low KRAKEN microbiome groups for both the blood and tumour samples the file Kraken-TCGA-Voom-SNM-Most-Stringent-Filtering-Data.csv was downloaded containing normalised log-cpm values, for each sample the rows were summed giving a overall ‘microbiome’ score. The samples were then divided into high and low groups based on the median of this score.

To investigate the role of any individual microbial species in influencing TCRA T cell fraction a reduced list of the species from the Kraken-TCGA-Voom-SNM-Most-Stringent-Filtering-Data.csv file were selected, by removing all species with less than 1000 total raw reads in the TCGA LUAD and LUSC cohort as called from the raw data file Kraken-TCGA-Raw-Data-17625-Samples.csv. This left a total of 59 microbial species that were individually tested for association with TCRA T cell fraction using Spearman's correlation for both LUAD and LUSC blood and tumour samples.

### Identification of Gain, Loss, and LOH Events in a Pan-Cancer Multi-Sample Cohort

Analysis of whole-exome sequencing was performed as described previously in Jamal-Hanjani et al. Copy-number segmentation, tumour purity and ploidy for each sample were estimated using ASCAT (Van Loo et al.) as described previously (Jamal-Hanjani et al.). These data were used as input to a multi-sample SCNA estimation approach to presence of loss of produce genome-wide estimates of the heterozygosity as well as loss, neutral, gain and amplification copy-number states relative to sample ploidy. The log ratio values present in each copy-number segment with 25 log ratio values in all samples of a tumour were examined relative to three sample-ploidy-adjusted log ratio thresholds using one-tailed t-tests with a P<0.01 threshold. These log ratio thresholds were equivalent to <log 2 [1.5/2] for losses, >log 2 [2.5/2] for gains in a diploid tumour. Any segment not classified as a loss or gain were classed as neutral. For each segment, these relative to ploidy definitions were combined with loss of heterozygosity detection across all samples from a single tumour.

### Pairwise Subclonal SCNA Scores

To calculate pairwise subclonal SCNA measures, the classifications outlined in the previous methods section were used to create three groups of pairwise subclonal SCNA scores. First, the inventors considered any segment affected by any of gain or loss relative to ploidy or LOH as aberrant and compared each pair of regions from a single patient's disease, classifying aberrant areas as clonal if aberrant in both samples or subclonal if aberrant in only one sample. This same process was repeated for gains relative to ploidy alone and then losses relative to ploidy and LOH considered together.

### Cytoband-Level SCNA Analysis

To enable comparisons across tumours, segments were mapped to hg19 cytobands. If multiple segments mapped to a cytoband, the SCNA status (gain or loss relative to ploidy) of the segment with the largest overlap with the cytoband was chosen. For the SCNA gain and loss analysis, cytoband level events were selected if they occurred subclonally across the entire cohort greater than 30 times. Bands passing this threshold within the same region (e.g. all cytobands on 1p36) were then grouped together. A Wilcoxon paired test was used to assess whether the tumour regions within a single patient with the subclonal SCNA events had a significant difference in TCRA T cell fraction to those regions without the event.

Selection of Multi-Sample Tumours with Heterogeneous Immune Infiltration

To be included a tumour had to have at least 3 regions sequenced and meet the following two requirements, 1) have a pair of regions with a large change in immune infiltration defined as having ≥0.1 difference in TCRA T cell fraction, and 2) have a pair of regions with a small or no change in immune infiltration defined as having <0.1 difference in TCRA T cell fraction. An example of a tumour matching this requirement would be one with three regions R1, R2 and R3 with TCRA T cell fractions of 0.01, 0.01 and 0.2 respectively. The R1-R2 pair has a difference in TCRA T cell fraction of 0 while the R1-R3 and R2-R3 pairs would both have a large difference of 0.19. Within the multi-sample tumour cohort 58 patients matched these criteria.

RNA-Seq Differential Gene Expression Analysis for Patients with Subclonal 12q24.31-32 Loss

Differential gene expression analysis was performed on the TRACERx100 RNA-seq patients with subclonal 12q24.31-32 loss. Using R 4.0.0, first the edgeR R package (version 3.32.1) was used for sample-specific TMM (Trimmed mean of M-values) normalisation, any genes with low expression were then filtered out using the standard edgeR filtering method before using the Limma-Voom method from the limma R package (version 3.46.0) to calculate the Voom fit and obtain p-values for the gene expression differences. The comparison controlled for patient and histology as blocking factors and p-values were FDR corrected for multiple testing. Results were then visualised with the R EnhancedVolcano package (version 1.8.0).

### Example 6-Estimation of T Cell Fraction in a Sample from WGS Data, Estimation of B Cell Fraction and Investigation of Immune Clonotypes

### Introduction

The approach described in Example 1 and exemplified in Examples 2-5 can be used to investigate further situations, such as where whole genome sequencing data is available, where other immune cell compartments than αβ T cells are of interest, and/or where usage of specific V and J segments is of interest such as e.g. to investigate clonotypes. In example, inventors this the demonstrate an implementation of the general approach described in Example 1 for WGS data. By utilising a subset of the TRACERx100 cohort with both WES and WGS data they validate the WGS version of the method showing high concordance between the use with WES data and the use with WGS data. They further provide an alternative model for the modelling of the read depth ratio (segment model) that takes into account the nature of V(D)J recombination making use of the greater coverage within WGS samples. They show using orthogonal RNA-seq data that the scores are accurately depicting the immune microenvironment and using down-sampling that the method remains accurate to as little as a depth of 2×. They then also show that the approach can be used to investigate the fraction of γδ T cells as well as TCR and BCR diversity. Finally, they also demonstrated the use of the method to study survival in a large pan-cancer cohort (data not shown).

### Results

Unlike whole exome sequencing (WES), WGS contains coverage across the entire genome. In the context of estimating copy number alterations, this provides a number of important advances, 1) uniform coverage allows for the accurate identification of the location of copy number breakpoints and 2) there are fewer biases such as those due to either GC content or reference allele bias. The inventors therefore reasoned that the T cell ExTRECT method described in Example 1 applied to WGS would have the potential to estimate T cell fraction to a greater accuracy at a lower depth. Additionally, six other genes TCRD, TCRG, TCRB, IGH, IGK and IGL (in addition to TCRA as demonstrated above) also undergo V(D)J recombination and have the potential to be quantified in the same way potentially allowing the estimation of αβ and γδ T cell compartment as well as B cells within a sample. This would be particularly beneficial as many WGS data sets lack accompanying immune data. The methods described herein could provide a comprehensive immune profile of a sample of any WGS sample without the need for any additional data.

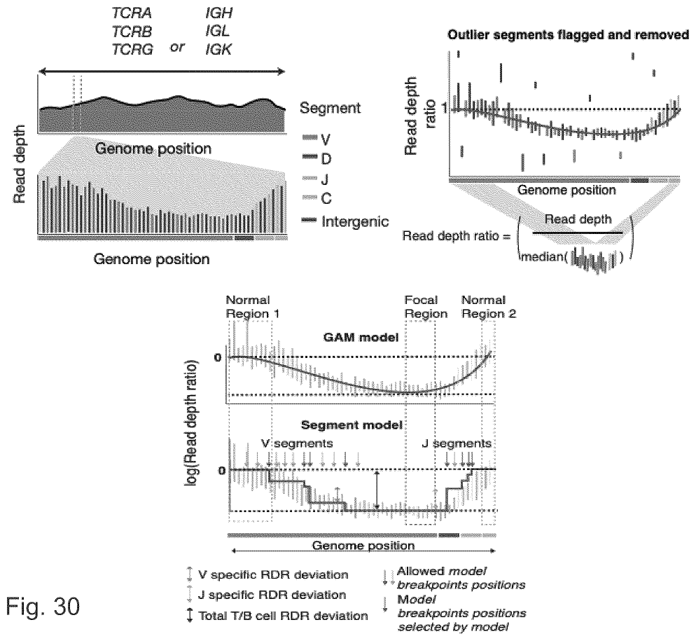

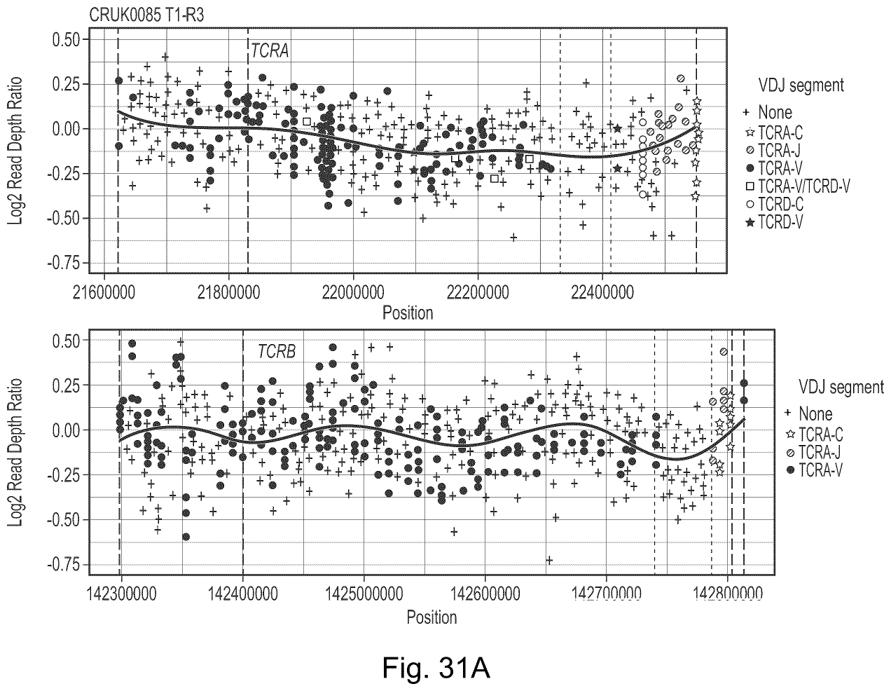

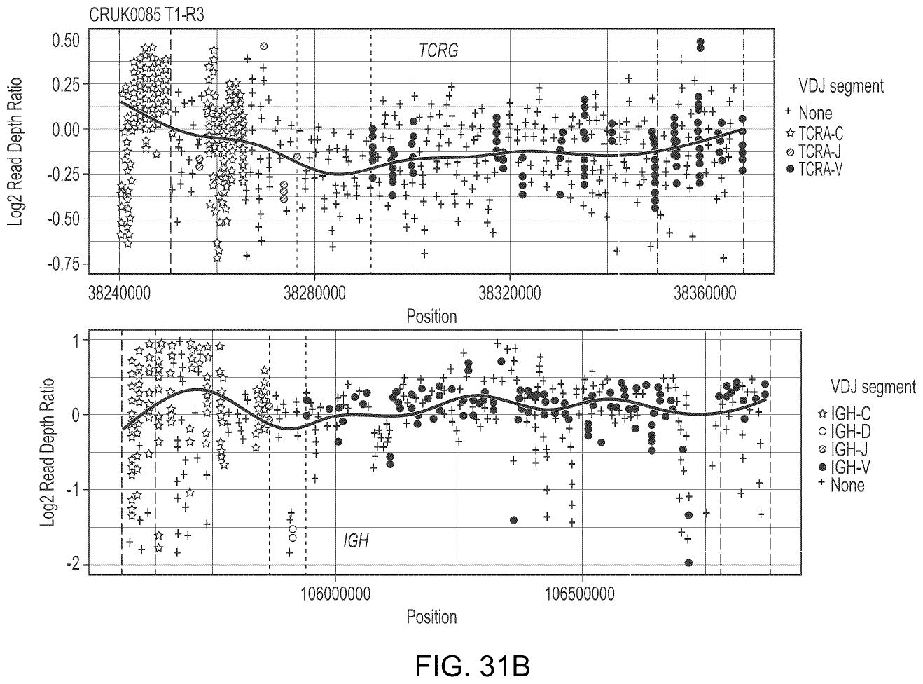

The method described above was adapted to WGS by applying the GC correction and quality control to 1000 bp windows instead of exon segments from WES capture kits and applying additional quality control to identify any 100 bp genomic window that had evidence of being an outlier in terms of having a much higher or lower read depth than nearby regions (see methods for full details). An overview of this adapted method (referred to as “adapted GAM”) is given in FIG. 30. Additionally, the inventors were able to apply the same WGS method to TCRB, TCRG and IGH by choosing different genomic loci for the focal and normalisation regions (see Table 7 for genomic locations used and plots of CRUK0085 T1-R3 as an example in FIG. 31). This particular data set was not amenable to exemplification of the method for IGL and IGK due to severe issues in quality of sequence alignment within these loci, presumably due to their high complexity and quality of the reference sequence resulting in difficulty mapping short read sequences in a uniform and non-bias way. Nevertheless, should a higher quality alignment be available, such as e.g. by making use of higher quality reference sequences, the same method should be applicable to these loci. Similarly, the method was not directly applied to TCRD due to the small size of its locus within TCRA. However, a segment usage based approach as described below was able to estimate the fraction of T cells including the TCRD segments.

### Benchmarking of Application to WGS Data

To take advantage of the uniform coverage in WGS data, an alternative model to the smooth general linear model (GAM) was designed. This is a piecewise constrained linear model where the breakpoints are forced to be located at the known locations of the V and J segments within the V(D)J locus. This “segment model” is described in the methods and FIG. 30 and has the following theoretical advantages over the GAM model: 1) it is based on real V(D)J recombination biology; 2) individual fractions can be calculated for the separate V and J gene segments giving an insight into segment usage and T or B cell receptor diversity and; 3) the fraction of the TCRD specific segments can be used as an indicator of gamma delta T cells. The model could be used with any sequencing data that has sufficient coverage throughout the region of interest. With the particular WES data this approach would not have been as advantageous (although it is theoretically possible) because the particular exon capture kits used have large gaps in coverage in the regions of interest. However, this may not be the case in other capture data sets.

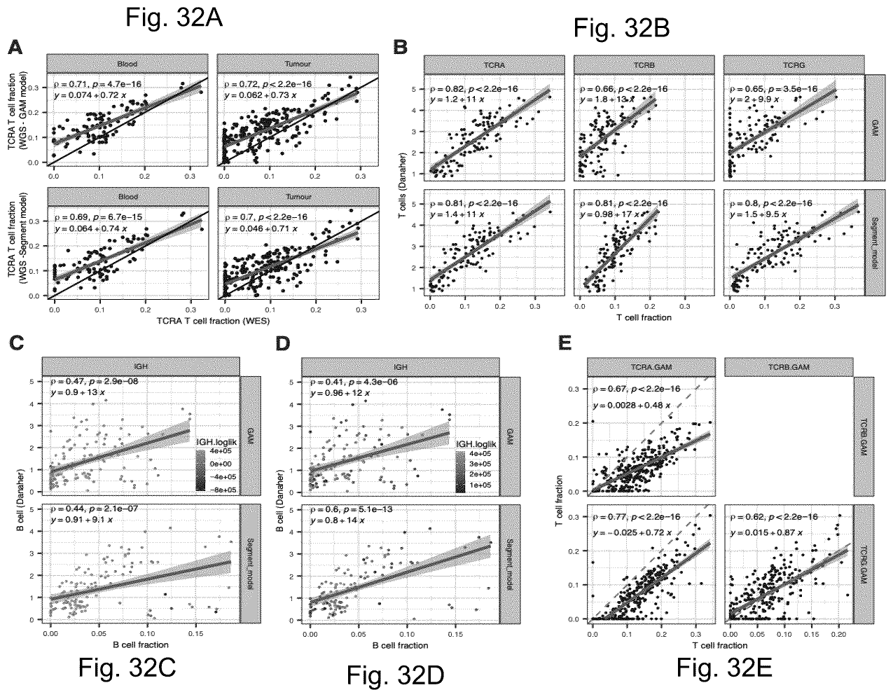

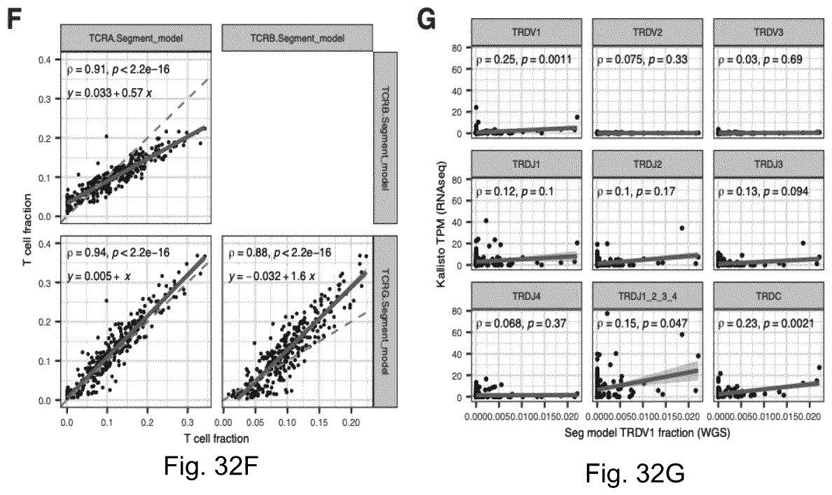

The accuracy of both the GAM model applied to WGS data and the segment model were tested by determining the T and B cell fractions on a set of 322 TRACERx100 samples for which matched WES and WGS was available, and 126 WGS samples with orthogonal RNAseq data. Comparing WES and WGS TCRA T cell fractions using the GAM model the inventors found a significant correlation in both the blood (ρ=0.71, P=4.7e−16, FIG. 32A) and tumour (ρ=0.72, P<2.2e−16, FIG. 32A) samples. Using the segment model the inventors found similar significant correlations with the GAM-based WES scores (blood: ρ=0.69, P=6.7e−15; tumour: ρ=0.7, P<2.2e−16, FIG. 32A). However, in both models the WGS values were found to be higher at lower T cell fractions, which may be due to the increased accuracy and sensitivity possible with WGS data. Supporting this when examining the orthogonal TRACERx100 RNAseq data a stronger correlation with the Danaher T cell scores was found with the WGS scores than the matched WES scores (WES: ρ=0.64, P=1.6e−15, (Danaher T cell score)=1.9+8.3 (ExTRECT T cell fraction (WES)), WGS (GAM): ρ=0.82, P<2.2e−16, WGS (segment model): ρ=0.81, P<2.2e−16, FIG. 32B).

### Validation of Approach for Other Loci

The Danaher scores calculated from the matched RNAseq data was used to orthogonally validate the accuracy of the T cell fractions calculated from TCRB and TCRG, and the B cell fraction calculated from IGH. Significant correlations with the T cell Danaher score were found for TCRB (GAM: ρ=0.66, P<2.2e−16; Segment model: ρ=0.81, P<2.2e−16, FIG. 32B) and TCRG (GAM: rho=0.64, P=3.1e−16; Segment model: rho=0.8, P<2.2e−16, FIG. 32C), and with the B cell Danaher score for IGH (GAM: ρ=0.47, P=2.3e−8; Seg model: ρ=0.44, P=2.1e−07 FIG. 32C). For the B cell fraction score from the segment model there were some clear outliers points with high B cell fraction but low B cell Danaher score. These scores tended to have very low log likelihood values from the segment model indicating a very noisy sample and a poor fit. Removing all samples with log likelihood <0 resulted in an improved correlation with the B cell Danaher score (GAM: ρ=0.41, P=4.3e−06; Segment model: ρ=0.6, P=5.1e−13). Overall these results demonstrate that the segment model produces scores that have stronger associations with the Danaher score than the corresponding values from the GAM model.

Comparing the different T cell fractions from TCRA, TCRB and TCRG, the TCRB T cell fraction was highly correlated to the TCRA T cell fraction but was roughly half its value in the GAM model (ρ=0.69, P <2.2 e−16, y=0.00059+0.49×, FIG. 32E) and slightly over half in the segment model (ρ=0.91, P<2.2e−16, y=0.033+0.57×, FIG. 32F). This is assumedly due to allelic exclusion with frequently only one of the TCRB alleles undergoing V(D)J recombination. In contrast TCRG T cell fraction was unexpectedly higher than the 1-5% of CD3+ T cells typically characterised as γδ T cells and found to be also highly correlated to the TCRA T cell fraction (GAM: ρ=0.71, P<2.2e−16, y=−0.028+0.71, FIG. 32E and segment model: ρ=0.94, P<2.2e−16, y=0.005+x). While there were a significant higher number of samples with close to 0 TCRG fraction compared to TCRA in the GAM model (32.8% of TCRG T cell fraction <1e−5 vs 1.7% TCRA T cell fraction <1e−5) the high fraction in the majority of samples and the near exact correlation between the two in the segment model would be widely considered biologically implausible if this signal was due completely to γδ T cells. Instead αβ T cells are known to initially undergo rearrangement of the TCRG locus before committing to their final lineage, the results from the segment model suggest that this it is a near universal feature of αβ T cells to have a rearranged TCRG locus and that the TCRG locus can provide an accurate measure of T cell fraction.

The TCRD locus is located entirely within the TCRA locus and too small in size to produce a score for fraction of γδ T cells accurately using the GAM model with the available data due to large amounts of noise.

Using the segment model it is however possible to calculate the fraction of T cells with segments coming from TCRD gene segments and thus calculate a T cell fraction arising from γδ T cells. The segment based score for TRDV1 was found to be significantly correlated to TPM RNAseq values for TCRD segments TRDV1 (ρ=0.25, P=0.0011, FIG. 32G), TRDJ1+TRDJ2+TRDJ3+TRDJ4 (ρ=0.15, P=0.047, FIG. 32G), TRDC (ρ=0.23, P=0.0021 FIG. 32G) in the TRACERx100 cohort.

### Investigation of Depth Requirements

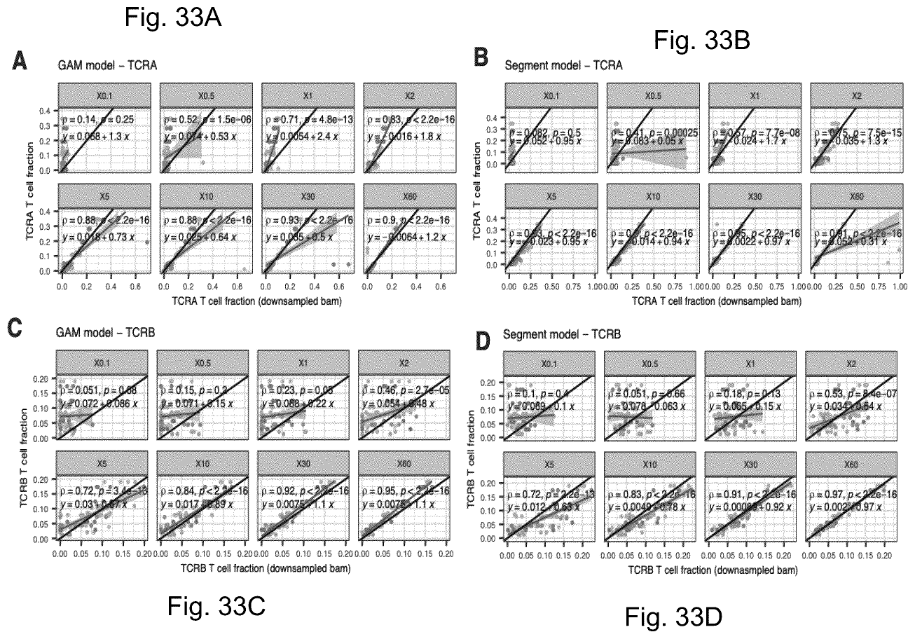

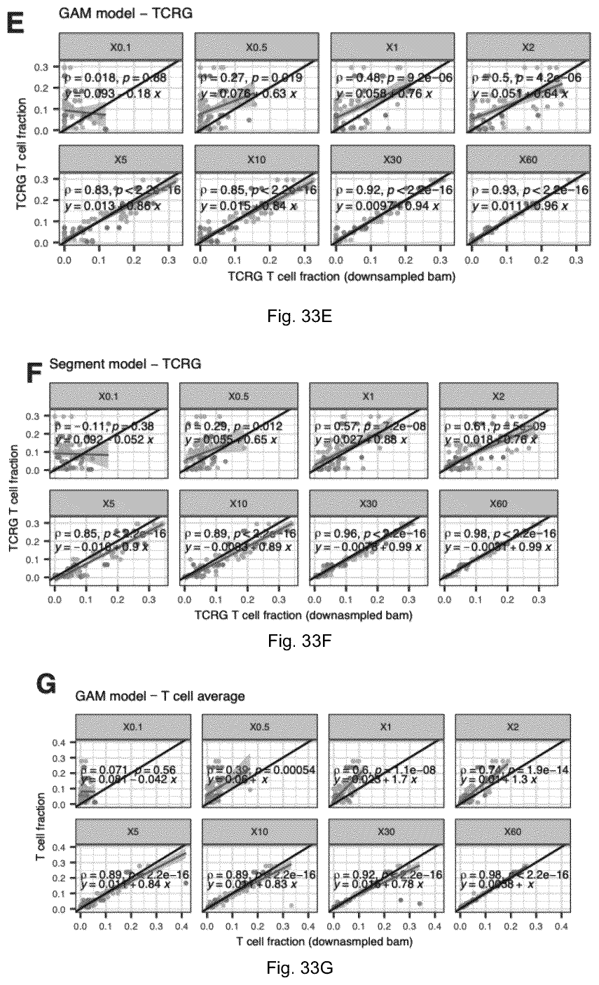

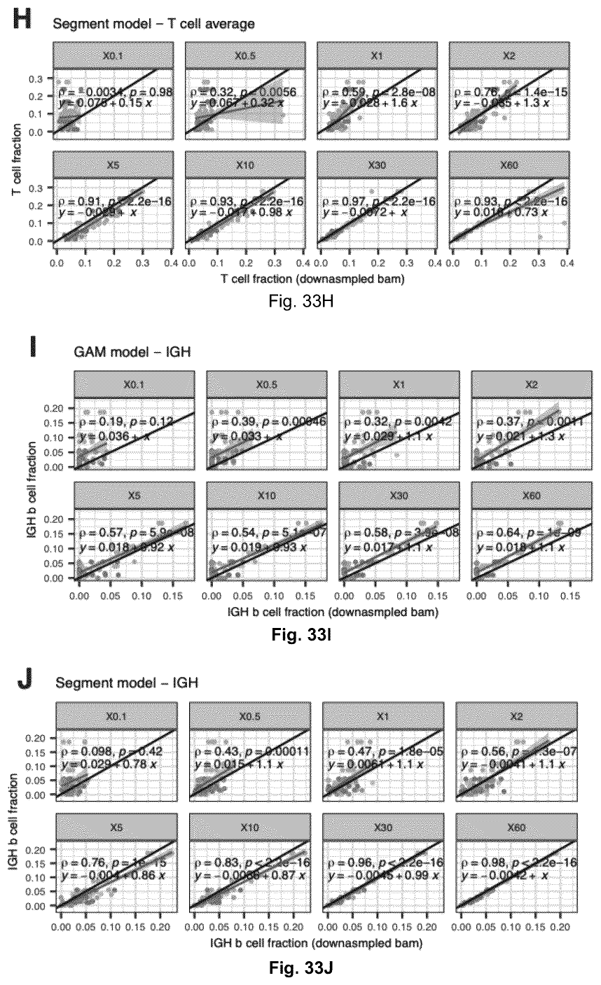

A subset of TRACERx100 samples (n=19) representing a range of T and B cell fractions from either the TCRA, TCRB, TCRG or IGH loci were selected for a downsampling analysis to ascertain the lowest depth that either the GAM or segment model was able to recapitulate the full depth scores. Using samtools each sample was downsampled randomly four times to a depth of either 60×, 30×, 10×, 5×, 2×, 1×, 0.5× or 0.1×. The results of this analysis are given in FIG. 33, and show high fidelity to the full depth values at 2× for both the GAM and segment model in all cases (TCRA GAM: ρ=0.83, P<2.2e−16, TCRA seg: ρ=0.75, P=7.5e−15, TCRB GAM: ρ=0.46, P=2.7e−05, TCRB seg: ρ=0.53, P=8.4e−07, TCRG GAM: ρ=0.5, P=4.2e−06, TCRG seg: ρ=0.61 P=5e−09, T cell average GAM: ρ=0.74, P=1.9e−14, T cell average seg: ρ=0.76 P=1.4e−15, IGH GAM: ρ=0.37, P=0.0011, IGH seg: ρ=0.56, P =1.3e−07, FIG. 33A-J) though at this low depth in many cases the values become deflated (e.g T cell average segment model at 1× has rho=0.59 but points lie on the line y=−0.028+1.6× when compared to the full depth values). Due to the observed high fidelity of scores at these low depth downsampled files, it is likely that by calculating the coverage in bins (such as e.g. bins from about 100 bp to about 10,000 bp, specifically values between 100 bp and 1000 bp would likely be particularly useful-appropriate values can be selected using a benchmarking approach as described above), the T cell ExTRECT method could be designed to work very well on low-pass WGS samples (such as e.g. 0.1× and above, between 0.1× and 0.5×, between 0.1× and 2×) with either the GAM or the segment model.

### The ExTRECT Approach can be Used to Investigate Immune Cell Repertoire Diversity

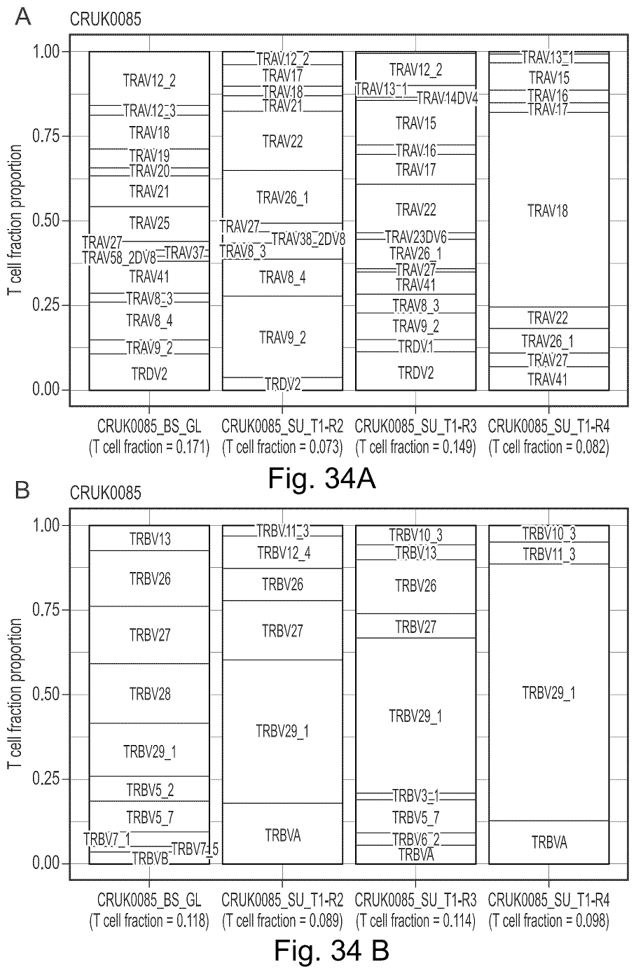

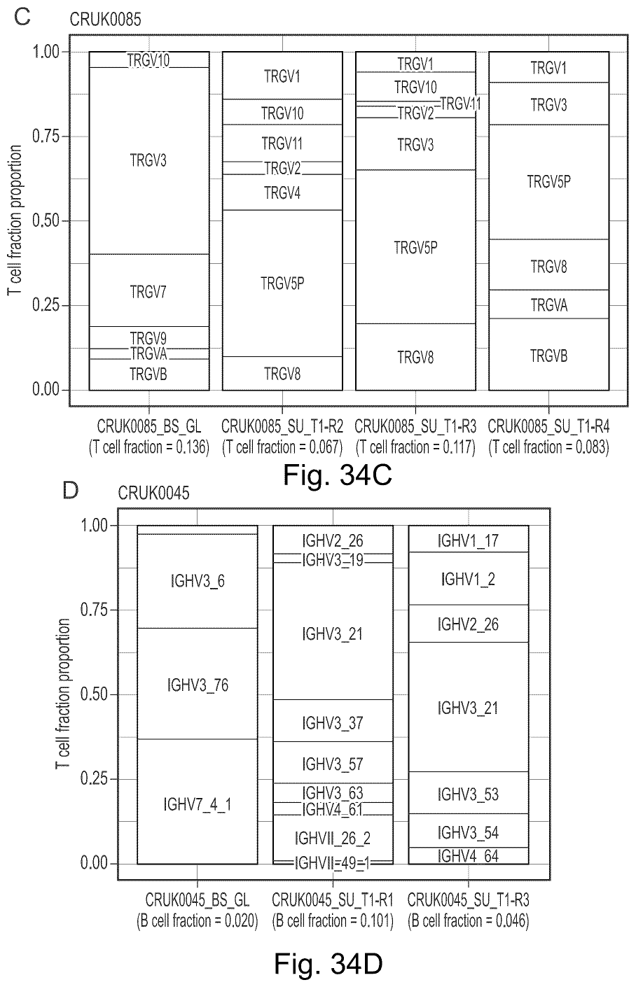

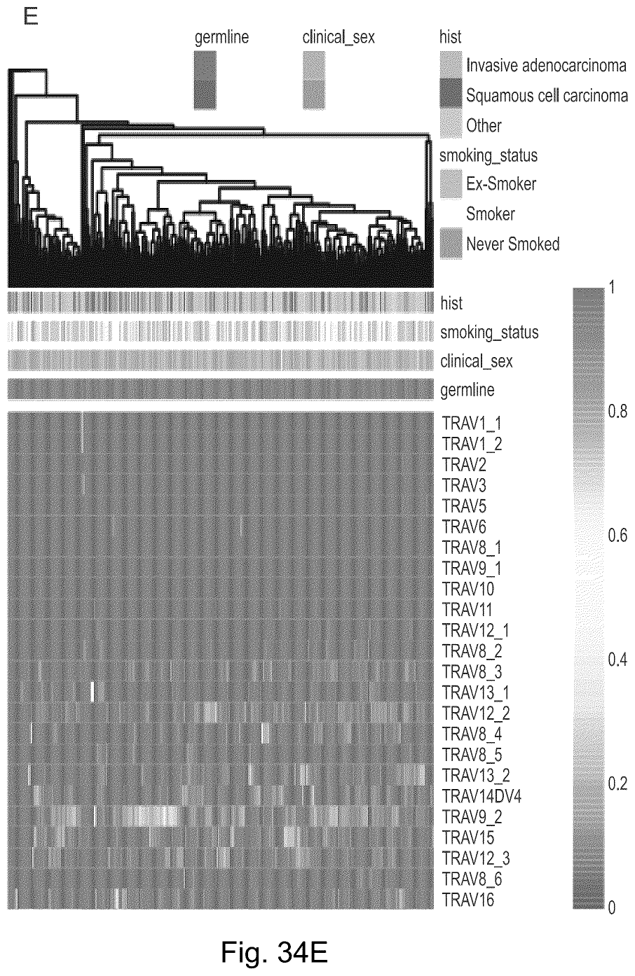

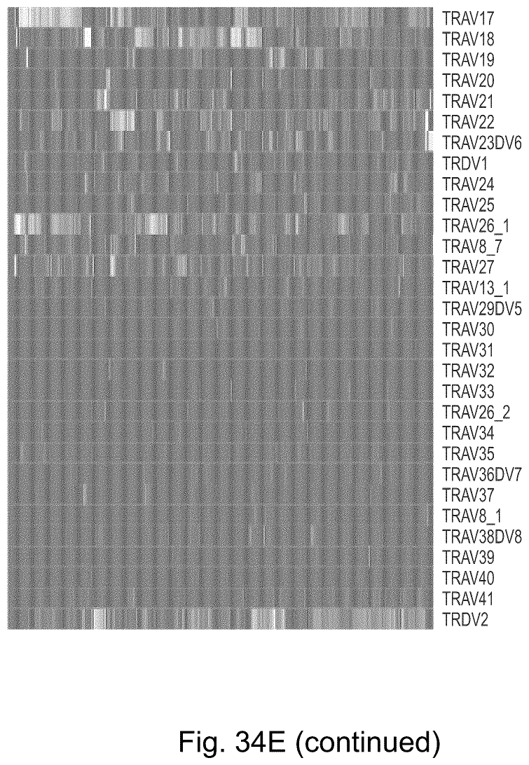

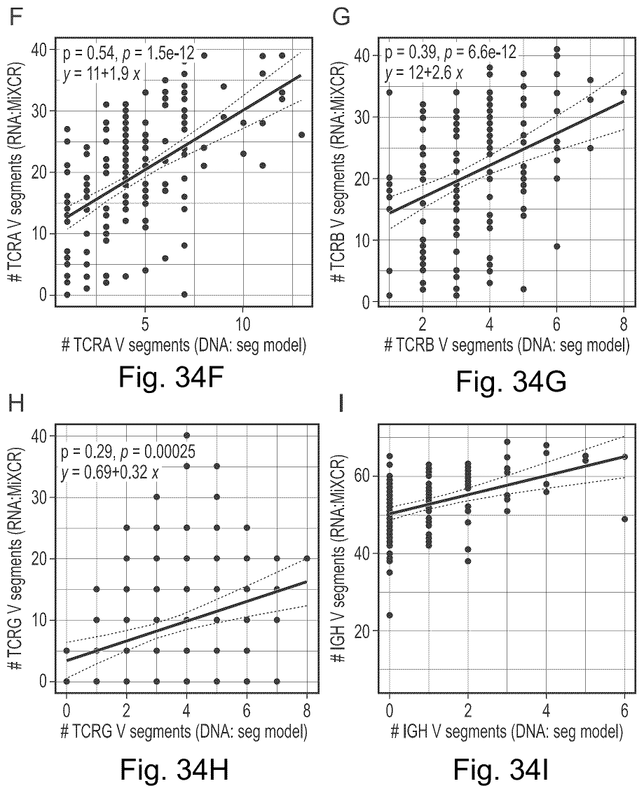

The use of the approach described above to give insight into more general TCR and BCR diversity was investigated. This was done within a sample by examining the V and J segment usage predicted from the optimal fit of the segment model. FIG. 34A-C show the proportion of V segments used in TRACERx patient CRUK0085 for TCRA, TCRB and TCRG across the different tumour regions and germline blood sample, and FIG. 34D shows the V segment usage for IGH in TRACERx patient CRUK0045. These can be obtained using the same equations for f as explained above based on the location of maximum recombination, but instead using the model estimate for the relevant segment as the value of r_{VDJ}. These clonotype plots show the potential of the method to identify samples with expanded clonotypes such as in the case of CRUK0085 where well over half of the T cells are predicted to use V segment TRAV18 in R4, as well as an increased proportion of TRBV29_1. Across an entire cohort it is possible to plot the changes in the proportions of segment usage, as shown in FIG. 34E with the TRAV segment. In this case despite a distinct clustering being formed there was no clear relationship with histology, clinical sex or whether the data was from a tumour or blood sample. Comparing the V segment called to those called from matched RNAseq data using bioinformatics software MiXCR (Bolotin et al, 2015) identified a significant correlation with the number of V segments called from T cell ExTRECT segment model (TCRA: ρ=0.54, P=1.5e−12, TCRB: ρ=0.39, P=6.6e−07, TCRG: ρ=0.29, P=0.00025, IGH: ρ=0.42, P=5.9e−08, FIG. 34F-I). The number of V segments called with MiXCR however is remarkably higher than the T cell ExTRECT model in all cases suggesting that the model being fit is oversimplified and not capturing the full TCR or BCR diversity. This may be addressed by expanding the V and J segments chosen from the model by taking a sample of the top fitting solutions instead of just the ‘optimal’ solution. For example, the top 10, 20, 30, 50 or 100 solutions (in terms of fit to the data) could be selected and investigated based on e.g. their confidence interval to select a model are capturing the full TCR or BCR diversity. These results particularly significant as some features of the TCR or BCR diversity have been shown to be indicative of prognosis and/or response to therapy in cancer. For example, B-cell gene expression signatures associated with various eve of diversity were found to be indicative of prognosis in breast and ovarian cancer (Iglesia et a., 2014), TCR repertoire analysis including determination of TCR baseline clonality and post treatment expansion was shown to be a source of biomarkers of response to checkpoint inhibitors therapy (Aversa et al, 2020, Hopkins et al., 2018), and TCR diversity in TIL at baseline was found to be prognostic in various cancers (Valpione et al., 2021).

### Association Between the Cancer Immune Environment and Survival, Clinical Determinants and Cancer Types in a Pan-Cancer Cohort

The T cell ExTRECT segment model was applied to a large pan-cancer WGS cohort consisting of >15000 participants across 20 different cancer types. This analyses provided T cell fractions derived from TCRA, TCRB and TCRG, and B cell fractions from IGH as well as fractions for individual V and J gene segments. The majority of these participants had a matched blood germline WGS sample with the remaining majority haematological cancer patients having matching germline samples from either saliva or other normal tissue. This allowed for the first time in a large pan-cancer cohort the opportunity to investigate differences in immune fraction in patient blood at the time of sampling.

This analysis revealed extensive diversity in the extent of immune landscape (TCRA T cell fraction, TCR diversity as measured by the number of TRAV segments call by the model with fractions over 0.01) both within and across cancer types, in blood and tumour samples. For both TCRA T cell fraction and the number of TRAV segments called in the segment model it is also possible to fit linear models to ascertain these values dependence on sex, age, and ancestry. These models can be fitted for blood TCRA T cell fraction and number of predicted TRAV segments, to investigate e.g. age is associated with decreasing T cell fraction and diversity in the pan-cancer cohort and whether this is observed to a different extent across the different cancer subtypes

Such an analysis can also be performed for the IGH B cell fraction score from T cell ExTRECT, investigating IGH B cell fraction in blood and tumour across different cancer types, as well as clinical determinants (e.g. sex, age, ancestry) of IGH B cell fraction and diversity.

To ascertain the germline and ancestry contribution to determining T cell fraction in blood, a GWAS analysis can be performed using PLINK and a set of unrelated patients amongst a cancer or pan-cancer cohort (see Methods). To increase power, a GWAS can be run separately for the blood TCRA, TCRB and TCRG T cell fraction values. These values are independently calculated from different regions of the genome and by looking at hits above the suggested level of significance (P<1e−5) in all three we can be confident that they are not the result of noise or artifacts within the data arising from SNPs within the V(D)J genes associated with reduction in mapping quality and coverage values (instead of real T cell fraction signal). Then, for any SNP identified in one or all three of these GWAS, it is possible to look at the enrichment of these SNPs in different cancer types.

Finally, survival data can be analysed, such as e.g. use data for date of cancer diagnosis, death records and latest follow up times from the most recent record in hospital episode statistics. Kaplan Meier curves can then be obtained for TCRA T cell and IGH B cell fraction in e.g. blood and tumour samples, dividing the cohort into high or low fraction in each case by the median value. This analysis can be used to investigate whether TCRA T cell fraction in blood/tumour or IGH B cell fraction in blood/tumour is significantly associated with survival

Finally, to better understand the relationship between immune cell fraction and other clinical features, as well as its heterogeneity across different cancer types, a Cox proportional model can be fitted composed of TCRA T cell fraction in blood and tumour, IGH B cell fraction in blood and tumour, and TRDV1 T cell fraction as a surrogate for gamma delta T cells in blood and tumour. The Cox model can also include age and sex, to control for possible confounding associations with T or B cell fraction, as well as whether the patient received chemotherapy pre-surgery. This final factor can be included as a surrogate for how aggressive or later stage the cancer was clinically. Alternative metrics such as cancer stage can be used. The results of such an analysis can be examined by looking a heatmap of the z scores from the Cox model run on any particular cohort. This can reveal whether TCRA T cell fraction in blood/tumour or IGH B cell fraction in blood/tumour is a significant risk in a pan-cancer cohort as well as any individual cancer types.

### Methods

The overall process to calculate the T cell fractions from WGS closely followed the method in Example 1. This includes using regions within the TCRA locus at the beginning and end as previously defined to normalise the coverage and calculate a single sample read depth ratio, as well as the formula in equations (3) or (7) for the T cell fraction, using γ=1 for Illumina HiSeq. The only differences reside in the details of calculation of r_{VDJ} (the deviation from 0 of the read depth ratio at the location of maximum V(D)J recombination). In particular, the GC correction method described in Example 1 was adapted to work on WGS instead of exons as described by a capture kit. The GC content was calculated at two scales, within 1000 bp windows (GC_{1000 bp}) and a smoothed version as calculated using a GAM model across all 1000 bp segments (GC_{macro}), as explained above. The coverage values were then normalised for GC content by fitting and taking the residuals from the following linear model:

$$lm ( basepair ∼ {GC}_{exon} + {GC}_{exon}^{2} + {GC}_{macro} + {GC}_{macro}^{2} )$$

This is identical to the one described in Example 1 except that the local GC content was calculated at the level of 1000 bp windows (GC_{1000 bp}) rather than at the level of individual exons (GCₑₓₒₙ).

As a final step a robust quality control process identified any 100 bp segments within any of the V (D) genes that were outliers in terms of coverage either 1) across the entire cohort or 2) associated with a subset of the cohort and linked to known germline genomic variants. For the first step the mean GC corrected ratio of all the V(D)J genes were calculated for the set of all the GEL Lung cohort. This revealed segments with either extremely low coverage compared to the regions surrounding them or extremely high coverage. By fitting a GAM model to the mean GC corrected ratio, those above and below a certain threshold from the fitted line (+/−0.25) could be excluded. This worked well for TCRA but for other genes such as TCRB there were large segments clustered together that interfered with the fit of the GAM model. For these some clear outlier regions were initially removed manually, e.g. all regions with mean GC ratio above 0.25 for TCRB. Following the removal of segments identified in this way, additional segments with biases were identified following a GWAS analysis using the PLINK software (see Renteria et al., 2013). In particular, a GWAS analysis was conducted to identify any SNPs associated with the T or B cell fraction. Regions of genes with bias in coverage in certain exons were identified and flagged for removal. The scores were recalculated and the GWAS process was repeated to check for removal of the bias. The removed segments had either under or over coverage relative to their surrounding region associated with a particular germline genotype and caused certain variants within the genes to be strongly associated with T or B cell fraction purely due to this artifact. In these cases, the specific samples with the associated genotype were selected and the average GC corrected ratio calculated. Additional outlier segments were then identified and flagged for removal by the same procedure as described above and the PLINK/GWAS analysis was rerun to assure that there were no further artifact segments.

The method was extended to other T cell receptor genes undergoing V(D)J recombination, TCRB and TCRG as well as B cell receptor IGH using the above method but redefining the locations used for normalisation of the read depth ratio as well as the location where maximum deviation is expected. In general the maximum deviation can be expected to be located between the final V segment and the first J segment and regions were chosen accordingly and are given in Table 7.

TABLE 7
Locations of regions (hg38) used for single sample
normalisation and calculation of r_{VDJ}

| | | | Maximum | |
|---|---|---|---|---|
| | | | V(D) J | |
| | | Normalisation | recombination | Normalisation |
| Gene | Chr | region 1 | region | region 2 |
| TCRA | 14 | 21621904- | 22331570- | 22547506- |
| | | 21830070 | 22411598 | 22752132 |
| TCRB | 7 | 142299011- | 142740894- | 142803288- |
| | | 142400160 | 142786879 | 142813287 |
| TCRG | 7 | 38240024- | 38276319- | 38350399- |
| | | 38250399 | 38291615 | 38368055 |
| IGH | 14 | 105566277- | 105865679- | 106779812- |
| | | 105633663 | 105939755 | 106879844 |

Similarly, the method was also applied to study segment usage, using the above method but using for the value of rVDJ the estimate from the segment model for the particular segment corresponding to the particular gene under investigation (in particular, the absolute value of the difference between the estimate from the segment model for the particular segment and the preceding segment—i.e. the drop or increase, depending on whether a V or J segment is considered, at the start of the segment under investigation, as illustrated on FIG. 31). Thus, the normalisation regions in Table 7 are used together with the segments equivalent to the “Maximum V(D)J recombination region” for each gene (provided as gene hgnc symbol-start_hg38-end_hg38) in Table 8 below. For the estimation of the fraction of γδ T cells, any of TRDV1, TRDV2, TRDJ1, TRDJ2, TRDJ3 and TRDJ4 can be used (all of which are located within the TCRA locus). The inventors found the V segment TRDV1 to most strongly correlate to RNAseq values related to γδ T cells. Thus, this segment was used for the estimation of the fraction of γδ T cells in this example. In this example, the segment model was used when studying segment usage, with the estimate from the model for the corresponding segment representing the value of r_{VDJ}. However, the same regions provided in Tables 7 and 8 can be used in combination with a GAM, preferably when coverage allows for studying of these regions, by estimating a summarised value for each of a plurality of segments to be investigated and determining the difference between consecutive segments as the r_{VDJ} for the second of a pair of consecutive segments.

TABLE 8
Locations of regions (hg38) used for single
segment calculation of r_{VDJ}

| | |
|---|---|
| TCRA | TRAV1-1-21621904-21622606; |
| segments | TRAV1-2-21642973-21643586; |
| | TRAV2-21712331-21712843; |
| | TRAV3-21723887-21724321; |
| | TRAV4-21736219-21736982; |
| | TRAV5-21749190-21749705; |
| | TRAV6-21768552-21769080; |
| | TRAV7-21782993-21783503; |
| | TRAV8-1-21797419-21797886; |
| | TRAV9-1-21811380-21812016; |
| | TRAV10-21825508-21826075; |
| | TRAV11-21829539-21830070; |
| | TRAV12-1-21841243-21841774; |
| | TRAV8-2-21846762-21847221; |
| | TRAV8-3-21852558-21853006; |
| | TRAV13-1-21868862-21869365; |
| | TRAV12-2-21887959-21888502; |
| | TRAV8-4-21894561-21895037; |
| | TRAV8-5-21903077-21904598; |
| | TRAV13-2-21918258-21918756; |
| | TRAV14DV4-21924138-21924651; |
| | TRAV9-2-21941184-21941657; |
| | TRAV15-21950127-21951654; |
| | TRAV12-3-21945507-21966061; |
| | TRAV8-6-21978679-21979120; |
| | TRAV16-21990496-21990938; |
| | TRAV17-21997686-21998168; |
| | TRAV18-22003211-22003673; |
| | TRAV19-22007629-22008181; |
| | TRAV20-22040663-22041153; |
| | TRAV21-22052521-22053056; |
| | TRAV22-22070690-22071208; |
| | TRAV23DV6-22086452-22086961; |
| | TRDV1-22096050-22096619; |
| | TRAV24-22105343-22105846; |
| | TRAV25-22112423-22113031; |
| | TRAV26-1-22123527-22124285; |
| | TRAV8-7-22132553-22133034; |
| | TRAV27-22148092-22148633; |
| | TRAV28-22155079-22155638; |
| | TRAV29DV5-22163349-22163870; |
| | TRAV30-22168429-22168988; |
| | TRAV31-22177273-22177864; |
| | TRAV32-22185562-22186061; |
| | TRAV33-22190158-22190714; |
| | TRAV26-2-22202583-22203368; |
| | TRAV34-22207534-22208129; |
| | TRAV35-22221896-22222475; |
| | TRAV36DV7-22226762-22227254; |
| | TRAV37-22265749-22267173; |
| | TRAV38-1-22271971-22272563; |
| | TRAV38-2DV8-22281151-22281748; |
| | TRAV39-22304054-22304553; |
| | TRAV40-22314490-22314919; |
| | TRAV41-22320188-22320691; |
| | TRDV2-22422546-22423042; |
| | TRDD1-22438547-22438554; |
| | TRDD2-22439007-22439015; |
| | TRDD3-22449113-22449125; |
| | TRDJ1-22450089-22450139; |
| | TRDJ4-22455249-22455296; |
| | TRDJ2-22456689-22456742; |
| | TRDJ3-22459098-22459156; |
| | TRDC-22462914-22466577; |
| | TRAJ61-22475316-22475375; |
| | TRAJ60-22476306-22476362; |
| | TRAJ58-22477707-22477769; |
| | TRAJ57-22478872-22478934; |
| | TRAJ56-22479521-22479582; |
| | TRAJ55-22481697-22481753; |
| | TRAJ54-22482287-22482346; |
| | TRAJ53-22483004-22483069; |
| | TRAJ52-22486228-22486296; |
| | TRAJ51-22487183-22487245; |
| | TRAJ50-22488593-22488652; |
| | TRAJ49-22489488-22489543; |
| | TRAJ48-22490491-22490553; |
| | TRAJ47-22492851-22492907; |
| | TRAJ46-22493403-22493465; |
| | TRAJ45-22493925-22493990; |
| | TRAJ44-22494821-22494883; |
| | TRAJ43-22495913-22495966; |
| | TRAJ42-22496887-22496952; |
| | TRAJ41-22497657-22497718; |
| | TRAJ40-22499689-22499749; |
| | TRAJ39-22501601-22501663; |
| | TRAJ38-22502231-22502292; |
| | TRAJ37-22503750-22503811; |
| | TRAJ36-22505110-22505168; |
| | TRAJ35-22506644-22506702; |
| | TRAJ34-22507666-22507723; |
| | TRAJ33-22508602-22508658; |
| | TRAJ32-22509341-22509406; |
| | TRAJ31-22510968-22511024; |
| | TRAJ30-22512852-22512908; |
| | TRAJ29-22513939-22513998; |
| | TRAJ28-22515623-22515688; |
| | TRAJ27-22516273-22516331; |
| | TRAJ26-22518446-22518505; |
| | TRAJ25-22518812-22518871; |
| | TRAJ24-22519969-22520031; |
| | TRAJ23-22520416-22520478; |
| | TRAJ22-22522040-22522102; |
| | TRAJ21-22523600-22523654; |
| | TRAJ20-22524325-22524381; |
| | TRAJ19-22525263-22525322; |
| | TRAJ18-22525650-22525715; |
| | TRAJ17-22526844-22526906; |
| | TRAJ16-22528527-22528586; |
| | TRAJ15-22529629-22529688; |
| | TRAJ14-22530327-22530378; |
| | TRAJ13-22531076-22531138; |
| | TRAJ12-22531939-22531998; |
| | TRAJ11-22532502-22532561; |
| | TRAJ10-22533497-22533560; |
| | TRAJ9-22535554-22535614; |
| | TRAJ8-22536145-22536204; |
| | TRAJ7-22537622-22537680; |
| | TRAJ6-22539073-22539134; |
| | TRAJ5-22540247-22540306; |
| | TRAJ4-22542199-22542261; |
| | TRAJ3-22543179-22543240; |
| | TRAJ2-22544071-22544136; |
| | TRAJ1-22545037-22545098; |
| | TRAC-22547506-22552132. |
| TCRB | TRBV1-142299177-142299460; |
| segments | TRBV2-142300924-142301432; |
| | TRBV3-1-142308542-142309048; |
| | TRBV4-1-142313184-142313666; |
| | TRBV5-1-142320677-142321544; |
| | TRBV6-1-142328297-142328786; |
| | TRBV7-1-142332182-142332701; |
| | TRBV4-2-142345421-142345985; |
| | TRBV6-2-142349152-142349664; |
| | TRBV7-2-142352819-142363358; |
| | TRBV8-1-142358639-142358917; |
| | TRBV5-2-142372640-142372912; |
| | TRBV6-4-142380806-142381261; |
| | TRBV7-3-142384329-142384841; |
| | TRBV8-2-142386378-142386647; |
| | TRBV5-3-142389202-142389668; |
| | TRBV9-142391891-142392412; |
| | TRBV10-1-142399860-142400377; |
| | TRBV11-1-142407672-142408136; |
| | TRBV12-1-142415224-142415666; |
| | TRBV10-2-142424965-142425465; |
| | TRBV11-2-142433895-142434394; |
| | TRBV12-2-142440883-142441325; |
| | TRBV6-5-142450947-142451448; |
| | TRBV7-4-142455174-142455635; |
| | TRBV5-4-142462916-142463581; |
| | TRBV6-6-142469537-142470013; |
| | TRBV7-5-142474096-142474567; |
| | TRBV5-5-142482548-142483019; |
| | TRBV6-7-142487863-142488295; |
| | TRBV7-6-142492132-142492673; |
| | TRBV5-6-142500028-142500534; |
| | TRBV6-8-142507382-142507810; |
| | TRBV7-7-142511626-142512127; |
| | TRBV5-7-142520090-142520556; |
| | TRBV7-9-142529290-142529762; |
| | TRBV13-142535809-142536292; |
| | TRBV10-3-142544212-142544685; |
| | TRBV11-3-142554836-142555318; |
| | TRBV12-3-142560423-142560931; |
| | TRBV12-4-142563740-142564245; |
| | TRBV12-5-142580917-142581427; |
| | TRBV14-142587868-142588359; |
| | TRBV15-142592928-142593473; |
| | TRBV16-142598016-142598469; |
| | TRBV17-142601628-142602360; |
| | TRBV18-142615716-142616415; |
| | TRBV19-142618849-142619532; |
| | TRBV20-1-142626649-142627399; |
| | TRBV21-1-142636924-142637384; |
| | TRBV22-1-142641746-142642196; |
| | TRBV23-1-142645961-142646467; |
| | TRBV24-1-142656701-142657213; |
| | TRBV25-1-142670740-142671244; |
| | TRBVA-142681415-142681869; |
| | TRBV26-142695699-142696183; |
| | TRBVB-142711384-142711924; |
| | TRBV27-142715346-142715861; |
| | TRBV28-142720660-142721160; |
| | TRBV29-1-142740206-142740894; |
| | TRBD1-142786213-142786224; |
| | TRBJ1-1-142786880-142786927; |
| | TRBJ1-2-142787017-142787064; |
| | TRBJ1-3-142787630-142787679; |
| | TRBJ1-4-142788225-142788275; |
| | TRBJ1-5-142788498-142788547; |
| | TRBJ1-6-142788988-142789040; |
| | TRBC1-142791694-142793368; |
| | TRBJ2-1-142796365-142796414; |
| | TRBJ2-2-142796560-142796610; |
| | TRBJ2-2P-142796697-142796742; |
| | TRBJ2-3-142796847-142796895; |
| | TRBJ2-4-142796998-142797047; |
| | TRBJ2-5-142797119-142797166; |
| | TRBJ2-6-142797239-142797291; |
| | TRBJ2-7-142797456-142797502; |
| | TRBC2-142801041-142802748; |
| | TRBV30-142812586-142813399. |
| TCRG | TRGC2-38239580-38249572; |
| segments | TRGJ2-38253380-38253429; |
| | TRGJP2-38256337-38256396; |
| | TRGC1-38257879-38265678; |
| | TRGJ1-38269491-38269540; |
| | TRGJP-38273587-38273647; |
| | TRGJP1-38276259-38276318; |
| | TRGV11-38291616-38292078; |
| | TRGVB-38295763-38296233; |
| | TRGV10-38299811-38300322; |
| | TRGV9-38317017-38318861; |
| | TRGVA-38322446-38322730; |
| | TRGV8-38330343-38330935; |
| | TRGV7-38335041-38335514; |
| | TRGV6-38340700-38340998; |
| | TRGV5P-38345030-38345499; |
| | TRGV5-38349355-38350022; |
| | TRGV4-38353715-38354517; |
| | TRGV3-38358512-38359162; |
| | TRGV2-38362864-38363518; |
| | TRGV1-38367586-38368169. |
| IGH | IGHA2-105583731-105588395; |
| segments | IGHE-105597691-105601728; |
| | IGHG4-105620506-105626066; |
| | IGHG2-105639559-105644790; |
| | IGHGP-105664633-105669843; |
| | IGHA1-105703995-105708665; |
| | IGHEP1-105721794-105722527; |
| | IGHG1-105736343-105743071; |
| | IGHG3-105764503-105771405; |
| | IGHD-105836765-105845677; |
| | IGHM-105851705-105856218; |
| | IGHJ6-105863198-105863258; |
| | IGHJ3P-105863416-105863465; |
| | IGHJ5-105863814-105863862; |
| | IGHJ4-105864215-105864260; |
| | IGHJ3-105864587-105864635; |
| | IGHJ2P-105864793-105864852; |
| | IGHJ2-105865199-105865250; |
| | IGHJ1-105865407-105865458; |
| | IGHD7-27-105865551-105865561; |
| | IGHJ1P-105865624-105865678; |
| | IGHD1-026-105881034-105881053; |
| | IGHD6-25-105881539-105881556; |
| | IGHD5-24-105883903-105883922; |
| | IGHD4-23-105884870-105884888; |
| | IGHD3-22-105886031-105886061; |
| | IGHD2-21-105888551-105888578; |
| | IGHD1-20-105891191-105891207; |
| | IGHD6-19-105891699-105891719; |
| | IGHD5-18-105893542-105893561; |
| | IGHD4-17-105894508-105894523; |
| | IGHD3-16-105895634-105895670; |
| | IGHD2-15-105897957-105897987; |
| | IGHD1-14-105900638-105900654; |
| | IGHD6-13-105901142-105901162; |
| | IGHD5-12-105902649-105902671; |
| | IGHD4-11-105903616-105903631; |
| | IGHD3-10-105904497-105904527; |
| | IGHD3-9-105904681-105904711; |
| | IGHD2-8-105907211-105907241; |
| | IGHD1-7-105909907-105909923; |
| | IGHD6-6-105910410-105910427; |
| | IGHD5-5-105912257-105912276; |
| | IGHD4-4-105913222-105913237; |
| | IGHD3-3-105914359-105914389; |
| | IGHD2-2-105916826-105916856; |
| | IGHD1-1-105919502-105919518; |
| | IGHV6-1-105939756-105940253; |
| | IGHVII-1-1-105945210-105945389; |
| | IGHV1-2-105986582-105987083; |
| | IGHVIII-2-1-106001534-106001824; |
| | IGHV1-3-106005095-106005574; |
| | IGHV4-4-106011922-106012420; |
| | IGHV7-4-1-106025145-106025630; |
| | IGHV2-5-106037902-106038365; |
| | IGHVIII-5-1-106039666-106039764; |
| | IGHVIII-5-2-106045600-106045860; |
| | IGHV3-6-106055549-106055998; |
| | IGHV3-7-106062151-106062683; |
| | IGHV3-64D-106088122-106088573; |
| | IGHV5-10-1-106107972-106108464; |
| | IGHV3-11-106116635-106117204; |
| | IGHVIII-11-1-106120207-106120473; |
| | IGHV1-12-106122420-106122709; |
| | IGHV3-13-106129540-106130072; |
| | IGHVIII-13-1-106142287-106142577; |
| | IGHV1-14-106145891-106146131; |
| | IGHV3-15-106153624-106154163; |
| | IGHVII-15-1-106163583-106163802; |
| | IGHV3-16-106165205-106165730; |
| | IGHVIII-16-1-106170749-106171052; |
| | IGHV1-17-106174342-106174760; |
| | IGHV1-18-106184901-106185394; |
| | IGHV3-19-106196700-106196990; |
| | IGHV3-20-106210936-106211453; |
| | IGHV3-21-106235064-106235594; |
| | IGHV3-11-106257762-106258223; |
| | IGHVII-22-1-106263362-106263626; |
| | IGHVIII-22-2-106264688-106264715; |
| | IGHV3-23-106268606-106269140; |
| | IGHV1-24-106276548-106277043; |
| | IGHV3-25-106289029-106289479; |
| | IGHVIII-25-1-106293568-106293808; |
| | IGHV2-26-106301396-106301862; |
| | IGHVIII-26-1-106309417-106309666; |
| | IGHVII-26-2-106314651-106314975; |
| | IGHV7-27-106317823-106318236; |
| | IGHV4-28-106324254-106324760; |
| | IGHVII-28-1-106329432-106329684; |
| | IGHV3-32-106331106-106331563; |
| | IGHV3-30-106335082-106335613; |
| | IGHVII-30-1-106342680-106342980; |
| | IGHV3-30-2-106344385-106344833; |
| | IGHV4-31-106349283-106349792; |
| | IGHVII-30-21-106354448-106354704; |
| | IGHV3-29-106356145-106356591; |
| | IGHV3-33-106359793-106360324; |
| | IGHVII-33-1-106367385-106367664; |
| | IGHV3-33-2-106369107-106369546; |
| | IGHV4-34-106373663-106374145; |
| | IGHV7-34-1-106377300-106377733; |
| | IGHV3-35-106389392-106389858; |
| | IGHV3-36-106392774-106393231; |
| | IGHV3-37-106396676-106397114; |
| | IGHV3-38-106410493-106411021; |
| | IGHVIII-38-1-106418018-106418299; |
| | IGHV4-39-106421711-106422218; |
| | IGHV7-40-106425359-106425654; |
| | IGHVII-40-1-106440950-106441007; |
| | IGHV3-41-106443133-106443583; |
| | IGHV3-42-106463256-106463691; |
| | IGHV3-43-106470264-106470800; |
| | IGHVII-43-1-106472791-106473000; |
| | IGHVIII-44-106478006-106478393; |
| | IGHVIV-44-1-106489345-106489756; |
| | IGHVII-44-2-106494134-106494383; |
| | IGHV1-45-106506996-106507491; |
| | IGHV1-46-106511117-106511856; |
| | IGHVII-46-1-106515818-106515854; |
| | IGHV3-47-106518582-106519027; |
| | IGHVIII-47-1-106531141-106531471; |
| | IGHV3-48-106537810-106538344; |
| | IGHV3-49-106556936-106557477; |
| | IGHVII-49-1-106564375-106564598; |
| | IGHV3-50-106566117-106566555; |
| | IGHV5-51-106578744-106579236; |
| | IGHV8-51-1-106583501-106583807; |
| | IGHVII-51-2-106584718-106584976; |
| | IGHV3-52-106586376-106586826; |
| | IGHV3-53-106592676-106593347; |
| | IGHVII-53-1-106599670-106599924; |
| | IGHV3-54-106601346-106601792; |
| | IGHV4-55-106606101-106606551; |
| | IGHV7-56-106609762-106610196; |
| | IGHV3-57-106618894-106619173; |
| | IGHV1-58-106622357-106622855; |
| | IGHV4-59-106627249-106627825; |
| | IGHV3-60-106631197-106631653; |
| | IGHVII-60-1-106637718-106637973; |
| | IGHV4-61-106639119-106639657; |
| | IGHV3-62-106643142-106643585; |
| | IGHVII-62-1-106650551-106650785; |
| | IGHV3-63-106652237-106652681; |
| | IGHV3-64-106657725-106658258; |
| | IGHV3-65-106666092-106666532; |
| | IGHVII-65-1-106671772-106672073; |
| | IGHV3-66-106675017-106675544; |
| | IGHV1-67-106680603-106681042; |
| | IGHVII-67-1-106686517-106686562; |
| | IGHVIII-67-2-106687113-106687200; |
| | IGHVIII-67-3-106692657-106692888; |
| | IGHVIII-67-4-106695102-106695397; |
| | IGHV1-68-106703846-106704286; |
| | IGHV1-69-106714684-106715181; |
| | IGHV2-70D-106723574-106724093; |
| | IGHV3-69-1-106728163-106728615; |
| | IGHV1-69-2-106737110-106737547; |
| | IGHV1-69D-106762092-106762588; |
| | IGHV2-70-106770577-106771020; |
| | IGHV3-71-106775157-106775618; |
| | IGHV3-72-106790694-106791233; |
| | IGHV3-73-106802694-106803233; |
| | IGHV3-74-106810442-106811131; |
| | IGHVII-74-1-106821174-106821414; |
| | IGHV3-75-106823687-106824147; |
| | IGHV3-76-106827869-106828163; |
| | IGHVIII-76-1-106831767-106832052; |
| | IGHV5-78-106851123-106851417; |
| | IGHVII-78-1-106865627-1068658985; |
| | IGHV3-79-106867660-106868092; |
| | IGHV4-80-106872783-106873186; |
| | IGHV7-81-106874583-106875071; |
| | IGHVIII-82-106879563-106879812. |

### Creation of New T Cell ExTRECT V(D)J Segment Model

The method described in Example 1 uses a GAM model to calculate r_{VDJ} and hence the T cell fraction within a sample. The GAM model simplifies modelling the process of V(D)J recombination as a smoothed process. This is useful when using WES data due to the non-uniformity of coverage with large segments of the genome within the TCRA locus not being sequenced by any exome capture kit. This comes at a cost of increased vulnerability to noise within the data. Further, this is not constrained to match the known biological reality of the process of V(D)J recombination. The benefits outweigh the costs in the case of WES data when the coverage within the locus or loci under investigation is not uniform. However, using WGS data (or any other data not suffering from this bias due to the particular regions captured) it is possible to use a model that is based directly on the biological process of V(D)J recombination. This was implemented by modelling the read depth ratio as a series of constant piecewise segments, with the location of breakpoints representing possible beginnings of deletion sites following V(D)J recombination, which are known from the location of the V and J gene segments and thus can be provided as constraints to the model. Thus, the inventors created a model where the segments are pre-chosen and align with the locations of the V and J segments. Additionally, the model can be constrained by the knowledge from V(D)J recombination that the read depth ratio should start from 0 and from there must be monotonically decreasing until the point of maximum V(D)J recombination. For example, in TCRA V(D)J recombination only some TCR chains will have selected the first TCRV-1 segment and all other V segments will be deleted, however all TCR chains will have a deletion following the final V segment. Likewise for J segments these must be monotonically increasing until reaching 0.

To fit this model, we transformed each possible segment with known break points corresponding to the V and J genes (see Table 8) into vectors of 1s and 0s, which are equal to 1 within their region and 0 outside. We then fit these vectors to the normalised read ratio data using a constrained linear model (using functions from the the R package restriktor v0.3), with inequality constraints chosen as follows for each of the n V segments and m J segments: V₁<0; V₂<V₁; . . . ; Vₙ<Vₙ₋₁; J₁>Vₙ; J₂>J₁; . . . ; Jₘ>Jₘ₋₁; Jₘ<0. Using these fitted values both the total T or B cell fraction can be calculated from the maximum deviation of the model e.g. the value from the last V segment as well as individual fractions from individual segments, e. g. segments relating to either TRDV1 or TRDV2 within the TCRA loci represent gamma delta T cells.

GWAS Analysis with PLINK

PLINK was run on a cohort of unrelated participants with WGS germline samples from blood and controlled with covariates for the first 20 genetic PCs, sex, age and disease type. The input of PLINK were the variants that had been pre-processed such that they had a MAF >0.001 in the entire cohort, passed QC filters including missingness and sufficient depth and underwent variant normalisation such as to make all multi-allelic variants bi-allelic as well as ensuring all variants were left aligned and parsimonious. In addition to these pre-set filters and QC before running PLINK we performed LD pruning in 500 kb windows with a R2 threshold of 0.2 as well as ensuring all variants had a MAF >0.001 in our cohort and a genotype missingness no greater than 0.2. Finally before running PLINK a Hardy-Weinberg test was performed with a threshold of 0.000001.

### Example 7—Discussion

The immune system is widely recognised as one of the key factors that influence both tumorigenesis and subsequent evolution of cancer. T cells play an important part of the cancer immune microenvironment by eliminating cancer cells that harbour neoantigens, and the number of T cells within a tumour sample is therefore an important clinical factor that may determine the course of disease. In the context of cancer, bulk DNA sequencing of tumour tissue has primarily been used to characterize the somatic alterations that drive tumour development. However, with the method presented in this paper we demonstrate that DNA sequencing can also be harnessed to study the immune microenvironment of a sample.

TRECs have been previously examined clinically in the context of screening for severe combined immune deficiency (SCID) in neonates (van der Spek, Groenwold, van der Burg, & van Montfrans, 2015) where their absence is used to infer T cell lymphopenia. However, these screening methods are based on the sequencing and quantification of TRECs themselves and are not based on WES data. A recent study of breast cancer single cell genome sequencing identified a deletion event in T cells (Baslan et al., 2020) within the TCRA gene. Here the inventors build upon this work to explicitly use TRECs to estimate T cell fraction in WES samples, as described in detail in Example 1.

The method described herein (TIL ExTRECT) provides an accurate estimate of immune infiltrate and its estimates show clinical utility. The inventors found that TCRA T cell fraction estimates are strongly correlated with orthogonal immune measures and that cancer cell lines and simulated WES data confirm their reliability (Example 2). Furthermore, the inventors demonstrated that the inferred TCRA T cell fraction is prognostic in LUAD and validate this finding in the TCGA LUAD cohort (Example 3). Relatedly, the inventors showed that the TCRA T cell fraction is associated with response to CPI in a pan-cancer cohort and improves upon the predictive value of clonal TMB alone (Example 4). The inventors further demonstrated that TCRA T cell fraction in blood and in tumour samples was predictive of survival in a pan-cancer cohort as well as in numerous specific cancer types, as was IGH B cell fraction (Example 6). The fraction of γδ T cells estimated based on the TRDV1 T cell fraction was found to be predictive of survival in specific cancer types.

In addition, TIL ExTRECT enables the T cell (or B cell) fraction to be calculated in datasets where this was previously not possible. Leveraging this, the inventors demonstrated that T cell fraction in blood is heterogeneous and is significantly higher in females than males, consistent with current findings and is also as expected associated with microbial infections (Example 5). Beyond recapitulating known immune associations we have shown that T cell ExTRECT can be used on data sets with no previous immune annotation. In a pan-cancer multi-sample cohort that lacked RNA-seq we observe a striking variation in the extent of heterogeneity in T cell infiltration across different cancer types (Example 5). Much of this heterogeneity appeared to be driven by subclonal SCNAs and they identified that subclonal loss of 12q24.31-32 was associated with significantly reduced T cell infiltrate. This may be linked to reduced expression of SPPL3 causing an upregulation of cell surface glycosphingolipids and thereby impeding the function of class I HLA molecules. This loss may be an immune escape mechanism in multiple cancer types, frequently occurring subclonally and therefore late in the evolutionary trajectory of a tumour.

In Example 2, the inventors have demonstrated that the TCRA T cell fraction is strongly correlated with orthogonal immune measures from different platforms and across multiple different datasets. Cancer cell lines and simulated WES data confirm that this score provides a reliable estimate for the fraction of T cells within a sequenced sample. The simulations and down sampling of high-depth TRACERx100 samples indicate 30× coverage provides sufficient signal to calculate a reliable T cell fraction (although lower coverages could be used to distinguish high and low T cell content, which may be useful as a more crude biomarker). This relatively low coverage means it should be applicable on most DNA sequencing datasets. In Example 6, the inventors demonstrate that the method can be applied to WGS data, where a signal with greater resolution than WES can be obtained outside of the TCRA region and even at lower depths. They show using orthogonal RNA-seq data that the scores are accurately depicting the immune microenvironment and using down-sampling that the method remains accurate to as little as a depth of 2×. In Example 6, they further demonstrate the potential of the method to investigate both gamma delta T cell fraction and both TCR and BCR diversity. Beyond WES and WGS, any NGS method that targets the TCRA gene (or any gene undergoing V(D)J recombination under investigation) is potentially compatible with this approach. Without wishing to be bound by theory, it is believed that sequencing data is preferable to e.g. SNP array data because the latter typically has significantly lower resolution in key areas of VDJ loci of interest. Additionally, a targeted panel-based sequencing approach could be used that would specifically target the TCRA gene (or any gene undergoing V(D)J recombination under investigation) and provide a TCRA T cell fraction (or TCRD, TCRG, TCRB, IGH, IGK or IGL cell fraction). Indeed, it is easy to envisage such an approach being combined with current panel-based sequencing approaches that estimate TMB in order to facilitate its use as a diagnostic tool. The sequencing platform specific constant (γ in equations (2), (3) and (7)—where a value of 1 is suitable for WES or WGS data) may be adjusted depending on the sequencing platform used. Further, platform-specific biases such as GC-content bias may be assessed and accounted for.

For these reasons, the TIL ExTRECT has potential value in the context of reanalysis of previously published NGS cancer datasets that otherwise lack unbiased estimates of T cell content. The TCRA T cell fraction also has the potential to be of value as a simple biomarker in the clinic due to its association with response to immunotherapy, as demonstrated in Example 4, and it association with survival as demonstrated in Example 6.

While there are other methods that provide immune related data from samples, often these are either unavailable or logistically cumbersome, for example imaging slides that require FFPE blocks to be retrieved, cut, stained before being manually or digitally evaluated. Meanwhile, T cell ExTRECT can be used in combination with DNA sequencing platforms that are commonly used in research and increasing the clinic.

While this method was developed within a cancer research framework, it is worth considering its application in a wider clinical setting. This measure can be calculated from any NGS analysis, taken from any tissue or blood in health or disease. The analysis of the matched blood samples within cancer cohorts demonstrated a significant relationship between the Blood TCRA T cell fraction with that of the patients' cancer type as well as sex shows the potential of this approach (see Example 3 and also Example 6). Further, the analysis of only the blood samples in the lung CPI cohort found the inferred TCRA T cell fraction to be predictive of response to immunotherapy without any analysis of the tumour samples themselves (FIG. 24B). Furthermore, non-NGS polymerase chain reaction (PCR) quantification of the levels of TRECs in blood samples has already been used to monitor thymic generation of T cells in trials of immunotherapy in breast cancer (Page et al.) and prostate cancer (Madan et al.). Additionally, T cell ExTRECT may have applications outside of oncology. One future clinical use of the Blood TCRA T cell fraction could be in the context of SCID (a group of rare congenital syndromes often resulting in severe T cell depletion for which treatment requires rapid haematopoietic stem cell transplantation), where TIL ExTRECT could be combined with genomic screening of neonates to identify potential damaging germline mutations and reduced T cell content simultaneously. Unfortunately, at the time of writing, SCID, though affecting 1 in 50,000 live births, is not part of routine neonatal screening in most countries and the majority of cases are diagnosed only after severe opportunistic infections and often too late for treatment (Chan et al.). TIL ExTRECT could potentially identify SCID by including TCRA within a newborn screening gene panel for genetic disease.

Since T cells and VDJ recombination are a defining feature of the adaptive immune system in jawed vertebrates, the general method presented herein is species agnostic. Although the TIL ExTRECT method described in the Examples above has been optimised for the human genome, this method can be extended to other species (primarily by defining the corresponding specific genomic regions to be used and assessing and addressing any region-specific bias) including much studied model organisms (including e.g. mouse, chicken, ferret etc.). It is also worth noting that VDJ recombination, while underpinning this novel approach to TCRA T cell fraction estimation, is not unique to the gene encoding the TCR-alpha receptor. The other TCR genes, beta, gamma and delta, also undergo VDJ recombination as do the BCR immunoglobulin genes IGH, IGL and IGK. Therefore this method can also be used to calculate the fraction of both alpha-beta, gamma-delta T cells as well as B cells with either IGL or IGK light chains, as demonstrated in Example 6. These may use appropriate chromosomal regions such as e.g. (hg19): TCRB chr7: 141998851-142510972; TCRG chr7: 38279625-38407656; IGH chr14: 106032614-107288051; IGL chr22: 22380474-23265085; IGK chr2: 89156674-90274235 or the regions in Tables 7 and 8; and assessing and addressing any region-specific bias. Some of these may not perform as well, depending on the noise in the data and the fraction of cells of interest in a sample, as these other immune cell types are typically less prevalent but, this extended method has the potential to give a detailed account of the immune microenvironment of a cancer sample utilising only DNA sequencing data (as demonstrated I Example 6).

In summary, the approach described herein, TIL ExTRECT, could have important applications in both basic and translational research by providing a cost-effective technique to characterise immune infiltrate alongside somatic changes in both human and model system datasets without the need for RNA sequencing.

### REFERENCES

AbdulJabbar et al. (2020). Geospatial immune variability illuminates differential evolution of lung adenocarcinoma. Nature Medicine, 26 (7), 1054-1062.
Aran, D., Hu, Z., & Butte, A. J. (2017). xCell: Digitally portraying the tissue cellular heterogeneity landscape. Genome Biology, 18 (1), 1-14.
Aversa I, Malanga D, Fiume G, Palmieri C. Molecular T-Cell Repertoire Analysis as Source of Prognostic and Predictive Biomarkers for Checkpoint Blockade Immunotherapy. Int J Mol Sci. 2020 Mar. 30; 21 (7): 2378.
Baslan et al. (2020). Novel insights into breast cancer copy number genetic heterogeneity revealed by single-cell genome sequencing. ELife, 9, 1-21.
Bolotin et al. (2012). Next generation sequencing for TCR repertoire Platform-specific features and correction algorithms. profiling: European Journal of Immunology, 42 (11), 3073-3083.
Bolotin, D. A. et al. MiXCR: software for comprehensive adaptive immunity profiling. Nat. Methods 12, 380-381 (2015).
Brastianos, P. K. et al. Genomic characterization of brain metastases reveals branched evolution and potential therapeutic targets. Cancer Discov. 5, 1164-1177 (2015).
Cancer Genome Atlas Research Network. (2012). Comprehensive genomic characterization of squamous cell lung cancers. Nature, 489 (7417), 519-525.
Cancer Genome Atlas Research Network. (2014). Comprehensive molecular profiling of lung adenocarcinoma. Nature, 511 (7511), 543-550.
Carter et al. (2012). Absolute quantification of somatic DNA alterations in human cancer. Nature Biotechnology, 30 (5), 413-421.
Chan, K. & Puck, J. M. Development of population-based newborn screening for severe combined immunodeficiency. J. Allergy Clin. Immunol. 115, 391-398 (2005).
Cristescu et al. (2018). Pan-tumor genomic biomarkers for PD-1 checkpoint blockade-based immunotherapy. Science, 362 (6411).
Danaher et al. (2018). Pan-cancer adaptive immune resistance as defined by the Tumor Inflammation Signature (TIS): Results from The Cancer Genome Atlas (TCGA). Journal for ImmunoTherapy of Cancer, 6 (1), 1-17.
Davoli, T., Uno, H., Wooten, E. C., & Elledge, S. J. (2017). Tumor aneuploidy correlates with markers of immune evasion and with reduced response to immunotherapy. Science, 355 (6322).
Denkert et al. (2016). Standardized evaluation of tumor-infiltrating lymphocytes in breast cancer: Results of the ring studies of the international immuno-oncology biomarker working group. Modern Pathology, 29 (10), 1155-1164.
Dieci et al. (2018) Update on tumor-infiltrating lymphocytes (TILs) in breast cancer, including recommendations to assess TILs in residual disease after neoadjuvant therapy and in carcinoma in situ: A report of the International Immuno-Oncology Biomarker Working Group on Breast Cancer. Seminars in Cancer Biology, Volume 52, Part 2, 2018, Pages 16-25.
Favero et al. (2015). Sequenza: Allele-specific copy number and mutation profiles from tumor sequencing data. Annals of Oncology, 26 (1), 64-70.
Galon et al. (2006). Type, density, and location of immune cells within human colorectal tumors predict clinical outcome. Science, 313 (5795), 1960-1964.
Gerlinger, M. et al. Intratumor Heterogeneity and Branched Evolution Revealed by Multiregion Sequencing. N. Engl. J. Med. 366, 883-892 (2012).
Gerlinger, M. et al. Genomic architecture and evolution of clear cell renal cell carcinomas defined by multiregion sequencing. Nat. Genet. 46, 225-233 (2014).
Ghandi et al. (2019). Next-generation characterization of the Cancer Cell Line Encyclopedia. Nature, 569 (7757), 503-508.
Goodman et al. (2017). Tumor mutational burden as an independent predictor of response to immunotherapy in diverse cancers. Molecular Cancer Therapeutics, 16 (11), 2598-2608.
Harbst, K. et al. Multiregion whole-exome sequencing uncovers the genetic evolution and mutational heterogeneity of early-stage metastatic melanoma. Cancer Res. 76, 4765-4774 (2016).
Haywood et al. Sunitinib's effect on tumor infiltration of CD8 T cells in renal cell carcinoma (RCC) and modulation of their function by altering VEGF-induced upregulation of PD1 expression. Journal of Clinical Oncology, 34 (2), 591 (2016).
Hendry, S., Salgado, R., Gevaert, T., Russell, P. A., John, T., Thapa, B., . . . Fox, S. B. (2017). Assessing Tumor-infiltrating Lymphocytes in Solid Tumors. Advances In Anatomic Pathology (Vol. 24).
Hopkins et al. T cell receptor repertoire features associated with survival in immunotherapy-treated pancreatic ductal adenocarcinoma. JCI Insight. 2018 Jul. 12; 3 (13): e122092.
Huang, W., Li, L., Myers, J. R., & Marth, G. T. (2012). ART: A next-generation sequencing read simulator. Bioinformatics, 28 (4), 593-594.
Hugo et al. (2016). Genomic and Transcriptomic Features of Response to Anti-PD-1 Therapy in Metastatic Melanoma. Cell, 165 (1), 35-44.
Iglesia et al. Prognostic B-cell signatures using mRNA-seq in patients with subtype-specific breast and ovarian cancer. Clin Cancer Res. 2014 Jul. 15; 20 (14): 3818-29.
Jamal-Hanjani et al. (2017). Tracking the Evolution of Non-Small-Cell Lung Cancer. New England Journal of Medicine, 376 (22), 2109-2121.
Jongsma et al. The SPPL3-Defined Glycosphingolipid Repertoire Orchestrates HLA Class I-Mediated Immune Responses. Immunity. 2021 Jan. 12; 54 (1): 132-150.e9. doi: 10.1016/j.immuni.2020.11.003. Epub 2020 Dec. 2. Erratum in: Immunity. 2021 Feb. 9; 54 (2): 387. PMID: 33271119.
Lamy, P. et al. Paired exome analysis reveals clonal evolution and potential therapeutic targets in urothelial carcinoma. Cancer Res. 76, 5894-5906 (2016).
Le, D. T., Uram, J. N., Wang, H., Bartlett, B. R., Kemberling, H., Eyring, A. D., . . . Diaz, L. A. (2015). PD-1 Blockade in Tumors with Mismatch-Repair Deficiency. New England Journal of Medicine, 372 (26), 2509-2520.
Levy, E., Marty, R., Gárate Calderón, V. et al. Immune DNA signature of T-cell infiltration in breast tumor exomes. Sci Rep 6, 30064 (2016).
Li et al. (2017). TIMER: A web server for comprehensive analysis of tumor-infiltrating immune cells. Cancer Research, 77 (21), e108-e110.
Litchfield et al. Meta-analysis of tumor and T cell-intrinsic mechanisms of sensitization to checkpoint inhibition. Cell 184.3 (2021): 596-614.
López et al. (2020). Interplay between whole-genome doubling and the accumulation of deleterious alterations in cancer evolution. Nature Genetics, 52 (3), 283-293.
Madan, R. A. et al. Clinical and immunologic impact of short-course enzalutamide alone and with immunotherapy in non-metastatic castration sensitive prostate cancer. J. Immunother. Cancer 9, (2021).
Mariathasan et al. (2018). TGFβ attenuates tumour response to PD-L1 blockade by contributing to exclusion of T cells. Nature, 554 (7693), 544-548.
Márquez et al. (2020). Sexual-dimorphism in human immune system aging. Nature Communications, 11 (1).
McDermott et al. (2018). Clinical activity and molecular correlates of response to atezolizumab alone or in combination with bevacizumab versus sunitinib in renal cell carcinoma. Nature Medicine, 24 (6), 749-757.
McGrath et al. Detecting T cell receptor rearrangements in silico from non-targeted DNA-sequencing (WGS/WES). bioRxiv preprint doi.org/10.1101/201947; Oct. 13, 2017.
Messaoudene, M. et al. T-cell bispecific antibodies in node-positive breast cancer: Novel therapeutic avenue for MHC class i loss variants. Ann. Oncol. 30, 934-944 (2019).
Middleton et al. (2020). The National Lung Matrix Trial of personalized therapy in lung cancer. Nature, 583 (7818), 807-812.
Newman et al. (2015). Robust enumeration of cell subsets from tissue expression profiles. Nature Methods, 12 (5), 453-457.
Newman et al. (2019). Determining cell type abundance and expression from bulk tissues with digital cytometry. Nature Biotechnology, 37 (7), 773-782.
Page, D. B. et al. A phase II study of dual immune checkpoint blockade (ICB) plus androgen receptor (AR) blockade to enhance thymic T-cell production and immunotherapy response in metastatic breast cancer (MBC). J. Clin. Oncol. 37, TPS1106-TPS1106 (2019).
Poore G D, Kopylova E, Zhu Q, et al. Microbiome analyses of blood and tissues suggest cancer diagnostic approach. Nature. 2020; 579 (7800): 567-574.
Racle et al. (2017). Simultaneous enumeration of cancer and immune cell types from bulk tumor gene expression data. ELife, 6, 1-25.
Riaz et al. (2017). Tumor and Microenvironment Evolution during Immunotherapy with Nivolumab. Cell, 171 (4), 934-949. e15.
Renteria M E, Cortes A, Medland S E. Using PLINK for Genome-Wide Association Studies (GWAS) and data analysis. Methods Mol Biol. 2013; 1019:193-213.
Riester, M., Singh, A. P., Brannon, A. R. et al. PureCN: copy number calling and SNV classification using targeted short read sequencing. Source Code Biol Med 11, 13 (2016).
Rizvi et al. (2015). Mutational landscape determines sensitivity to PD-1 blockade in non-small cell lung cancer. Science, 348 (6230), 124-128.
Robert et al. (2011). Ipilimumab plus Dacarbazine for Previously Untreated Metastatic Melanoma. New England Journal of Medicine, 364 (26), 2517-2526.
Rosenthal et al. (2019). Neoantigen-directed immune escape in lung cancer evolution. Nature, 567 (7749), 479-485.
Samstein et al. (2019). Tumor mutational load predicts survival after immunotherapy across multiple cancer types. Nature Genetics, 51 (2), 202-206.
Savas, P. et al. The Subclonal Architecture of Metastatic Breast Cancer: Results from a Prospective Community-Based Rapid Autopsy Program “CASCADE”. PLOS Med. 13, 1-25 (2016).
Schadendorf et al. (2015). Pooled analysis of long-term survival data from phase II and phase III trials of ipilimumab in unresectable or metastatic melanoma. Journal of Clinical Oncology, 33 (17), 1889-1894.
Shen, R., & Seshan, V. (2015). FACETS: Fraction and Allele-Specific Copy Number Estimates from Tumor Sequencing. Memorial Sloan-Kettering Cancer Center, Dept. of Epidemiology & Biostatistics Working Paper Series., 1, 50. Retrieved from http://biostats.bepress.com/mskccbiostat/paper29
Shim et al. (2020). HLA-corrected tumor mutation burden and homologous recombination deficiency for the prediction of response to PD-(L) 1 blockade in advanced non-small-cell lung cancer patients. Annals of Oncology, 31 (7), 902-911.
Snyder et al. (2014). Genetic Basis for Clinical Response to CTLA-4 Blockade in Melanoma. New England Journal of Medicine, 371 (23), 2189-2199.
Snyder et al. (2017). Contribution of systemic and somatic factors to clinical response and resistance to PD-L1 blockade in urothelial cancer: An exploratory multi-omic analysis. PLOS Medicine, 14 (5), 1-24.
Suzuki, H. et al. Mutational landscape and clonal architecture in grade II and III gliomas. Nat. Genet. 47, 458-468 (2015).
Thorsson et al. (2018). The Immune Landscape of Cancer. Immunity, 48 (4), 812-830.e14.
Topalian et al. (2012). Safety, Activity, and Immune Correlates of Anti-PD-1 Antibody in Cancer. New England Journal of Medicine, 366 (26), 2443-2454.
Turajlic, S. et al. Deterministic Evolutionary Trajectories Influence Primary Tumor Growth: TRACERx Renal. Cell 173, 595-610. e11 (2018).
Valpione, S., Mundra, P.A., Galvani, E. et al. The T cell receptor repertoire of tumor infiltrating T cells is predictive and prognostic for cancer survival. Nat Commun 12, 4098 (2021).
Van Allen et al. (2016). Erratum for the report “genomic correlates of response to CTLA-4 blockade in metastatic melanoma.” Science, 352 (6283), 207-212.
van der Spek, J., Groenwold, R. H. H., van der Burg, M., & van Montfrans, J. M. (2015). TREC Based Newborn Screening for Severe Combined Immunodeficiency Disease: A Systematic Review. Journal of Clinical Immunology, 35 (4), 416-430.
Van Loo et al. (2010). Allele-specific copy number analysis of tumors. Proceedings of the National Academy of Sciences of the United States of America, 107 (39), 16910-16915.
Wang et al. (2007). PennCNV: An integrated hidden Markov model designed for high-resolution copy number variation detection in whole-genome SNP genotyping data. Genome Research, 17 (11), 1665-1674.
Wang, V. G., Kim, H., & Chuang, J. H. (2018). Whole-exome sequencing capture kit biases yield false negative mutation calls in TCGA cohorts. PLOS ONE, 13 (10), 1-14.
Watkins, T. B. K. et al. Pervasive chromosomal instability and karyotype order in tumour evolution. Nature 587, 126-132 (2020).
Yokoyama, A., Kakiuchi, N., Yoshizato, et al. T. Age-related remodelling of oesophageal epithelia by mutated cancer drivers. Nature 565, 312-317 (2019).
Yoshihara et al. (2013). Inferring tumour purity and stromal and immune cell admixture from expression data. Nature Communications, 4.
Zaccaria, S., & Raphael, B. J. (2020). Accurate quantification of copy-number aberrations and whole-genome duplications in multi-sample tumor sequencing data. Nature Communications, 11 (1).
Zhang et al. (2003). Intratumoral T Cells, Recurrence, and Survival in Epithelial Ovarian Cancer. New England Journal of Medicine, 348 (3), 203-213.
Wood, D. E. & Salzberg, S. L. Kraken: Ultrafast metagenomic sequence classification using exact alignments. Genome Biol. 15, (2014).

All references cited herein are incorporated herein by reference in their entirety and for all purposes to the same extent as if each individual publication or patent or patent application was specifically and individually indicated be incorporated by reference in its entirety.

This application claims priority from GB application No. 2110555.6 filed 22 Jul. 2021 and GB application 2203451.6 filed 11 Mar. 2022. The entire content of both priority applications is incorporated herewith by reference for all purposes.

The specific embodiments described herein are offered by way of example, not by way of limitation. Various modifications and variations of the described compositions, methods, and uses of the technology will be apparent to those skilled in the art without departing from the scope and spirit of the technology as described. Any sub-titles herein are included for convenience only, and are not to be construed as limiting the disclosure in any way.

The methods of any embodiments described herein may be provided as computer programs or as computer program products or computer readable media carrying a computer program which is arranged, when run on a computer, to perform the method(s) described above.

Unless context dictates otherwise, the descriptions and definitions of the features set out above are not limited to any particular aspect or embodiment of the invention and apply equally to all aspects and embodiments which are described.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example+/−10%.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

Other aspects and embodiments of the invention provide the aspects and embodiments described above with the term “comprising” replaced by the term “consisting of” or “consisting essentially of”, unless the context dictates otherwise.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

1. A method for determining the fraction of lymphocytes in a mixed sample comprising genomic material from multiple cell types, the method comprising:
obtaining a read depth profile for the sample comprising the read depths along a predetermined genomic region of interest, wherein the predetermined genomic region of interest includes at least a portion of a genomic locus that undergoes VDJ recombination;
obtaining a plurality of read depth ratios (rᵢ) by normalising the read depths along the predetermined region of interest by reference to a baseline read depth derived from a subset of the region of interest;
obtaining a summarised read depth ratio value (r_{VDJ}) for a subset of the region of interest that is likely to be deleted through VDJ recombination; and
determining the fraction of lymphocytes (f) in the sample as a function of the summarised read depth ratio value (r_{VDJ}).

2. The method of claim 1, wherein a genomic locus that undergoes VDJ recombination is selected from the TCRA, TCRB, TCRG, TCRD, IGH, IGL or IGK gene locus, and/or wherein the fraction of lymphocytes is a fraction of T lymphocytes, a fraction of B lymphocytes, a fraction of αβ T lymphocytes, a fraction of γδ T lymphocytes, a fraction of B cells or T cells including any gene segment in the TCRA, TCRB, TCRG, TCRD, IGH, IGL or IGK gene locus, or a fraction of B cells or T cells including any gene segments listed in Table 8.

3. The method of claim 2, wherein the predetermined region of interest includes one or more exons from the TCRA, TCRB, TCRG, TCRD, IGH, IGL or IGK gene locus.

4. The method of claim 2 or claim 3, wherein the predetermined region of interest includes a plurality of exons from the TCRA, TCRB, TCRG, TCRD, IGH, IGL or IGK gene locus, wherein the plurality of exons comprise at least one exon corresponding to each of a V, D and C segment of the respective gene locus.

5. The method of claim 4, wherein the plurality of exons comprise exons corresponding to multiple V segments of the respective gene locus.

6. The method of any preceding claim, wherein a read depth profile is obtained from sequencing data, preferably from whole exome sequencing, whole genome sequencing, or panel sequencing data.

7. The method of any preceding claim, wherein determining the fraction of lymphocytes (f) in the sample as a function of the summarised read depth ratio value (r_{VDJ}) comprises determining the fraction of lymphocytes (f) in the sample as a function of the summarised read depth ratio value (r_{VDJ}), the fraction of abnormal cells (p) in the mixed sample, where abnormal cells are those that are aneuploid in the predetermined region of interest, and the copy number of the abnormal cells in the predetermined region of interest (VT).

8. The method of claim 7, wherein determining the fraction of lymphocytes (f) in the sample comprises determining the value of f as:
$$f = ( 1 - p + \frac{p}{2} {Ω}_{T} ) - ( 1 - p + \frac{p}{2} {Ψ}_{T} ) {2}^{\frac{{r}_{VDJ}}{γ}} \qquad ( 7 )$$
where γ is a constant.

9. The method of any preceding claim, wherein determining the fraction of lymphocytes (f) in the sample comprises determining the value of f as:
$$f = 1 - {2}^{\frac{{r}_{VDJ}}{γ}} \qquad ( 3 )$$
where γ is a constant.

10. The method of any preceding claim, wherein the subset of the region of interest that is likely to be deleted through VDJ recombination comprises a region located between the end of the last V segment of the genomic locus that undergoes VDJ recombination, and the start of the J segment of the genomic locus that undergoes VDJ recombination, preferably wherein the subset of the region of interest that is likely to be deleted through VDJ recombination comprises one or more D segments (such as e.g. all D segments) of the genomic locus that undergoes VDJ recombination.

11. The method of any preceding claim, further comprising fitting a model to the plurality of read depth ratios, and wherein obtaining a summarised read depth ratio value (r_{VDJ}) for a subset of the region of interest that is likely to be deleted through VDJ recombination comprises obtaining a summarised read depth ratio value based on the value of the model in the subset of the region of interest that is likely to be deleted through VDJ recombination, optionally wherein the model is a general linear model, a piecewise constrained linear model with a plurality of breakpoints selected to correspond to locations of a plurality of V and J genes in the region of interest, or a Bayesian model modelling segment usage from the read depth and a prior distribution of usage of each of a plurality of V and J genes in the region of interest.

12. The method of any preceding claim, further comprising obtaining a baseline read depth from a subset of the region of interest, preferably wherein the baseline read depth is a summarised read depth value across the said subject of the region of interest.

13. The method of any preceding claim, wherein the subset of the region of interest used to obtain the baseline read depth is a subset of the region of interest that is unlikely to be deleted through VDJ recombination.

14. The method of claim 13, wherein the subset of the region of interest that is unlikely to be deleted through VDJ recombination comprise the n first V segments of the genomic locus that undergoes VDJ recombination, and/or wherein the subset of the region of interest that is unlikely to be deleted through VDJ recombination comprises the C segment of the genomic locus that undergoes VDJ recombination.

15. The method of any preceding claim, wherein obtaining a read depth profile comprises obtaining a plurality of raw read depth values across the predetermined region of interest and smoothing the read depth values, optionally wherein smoothing the read depth values comprises replacing the raw read depth values by a corresponding rolling median over a window of a predetermined width.

16. The method of any preceding claim, wherein the mixed sample is a blood sample or a tumour sample, and/or wherein the method further comprises obtaining a mixed sample comprising genomic material from multiple cell types, from a subject, and/or obtaining read depth data from a mixed sample comprising genomic material from multiple cell types, from a subject through one or more in vitro steps.

17. The method of any preceding claim, further comprising providing to a user, for example through a user interface, the determined lymphocyte fraction and/or any value derived therefrom.

18. A method of providing a prognosis for a subject that has been diagnosed as having cancer, the method comprising determining the lymphocyte fraction in one or more tumour samples from the subject using the method of any of claims 1 to 17.

19. A method of determining whether a subject that has been diagnosed as having cancer is likely to respond to immunotherapy, the method comprising determining the lymphocyte fraction in one or more tumour samples from the subject using the method of any of claims 1 to 17.

20. A method of determining whether a subject has T cell lymphopenia, the method comprising determining the lymphocyte fraction in one or more samples from the subject using the method of any of claims 1 to 17.

21. A system comprising:
a processor; and
a computer readable medium comprising instructions that, when executed by the processor, cause the processor to perform the steps of the method of any of claims 1 to 15 or 17 to 20.
